(19) United States
(12) Patent Application Publication
(10) Pub. No.: US 2023/0024438 A1
(43) Pub. Date: Jan. 26, 2023

(54) INHIBITORS OF HIF-2ALPHA

(71) Applicant: ARCUS BIOSCIENCES, INC., Hayward, CA (US)

(72) Inventors: Joel Worley BEATTY, San Mateo, CA (US); Samuel Lawrie DREW, Millbrae, CA (US); Jeremy Thomas Andre FOURNIER, Fremont, CA (US); Tezcan GUNEY, Hayward, CA (US); Steven Donald JACOB, Oakland, CA (US); Kenneth Victor LAWSON, San Francisco, CA (US); Manmohan Reddy LELETI, Dublin, CA (US); Erick Allen LINDSEY, San Diego, CA (US); Debashis MANDAL, Fremont, CA (US); Guillaume MATA, Berkeley, CA (US); Jay Patrick POWERS, Sisters, OR (US); Brandon Reid ROSEN, San Mateo, CA (US); Yongli SU, Foster City, CA (US); Anh Thu TRAN, Union City, CA (US); Xuelei YAN, Foster City, CA (US)

(21) Appl. No.: 17/777,103

(22) PCT Filed: Dec. 3, 2020
(86) PCT No.: PCT/US2020/063000

§ 371 Date: May 16, 2022

Related U.S. Application Data
(60) Provisional application No. 62/943,632, filed on Dec. 4, 2019.

(51) Int. Cl. C07D 231/56; C07D 401/12; C07D 403/12; A61K 39/395; A61K 31/497; A61K 31/4439; A61K 31/416; C07D 471/04; C07D 487/04; C07D 401/04; A61K 31/4709; A61K 31/437; A61K 31/519

(52) U.S. Cl. CPC C07D 231/56; C07D 401/12; C07D 403/12; A61K 39/3955; A61K 31/497; A61K 31/4439; A61K 31/416; C07D 471/04; C07D 487/04; C07D 401/04; A61K 31/4709; A61K 31/437; A61K 31/519

(57) ABSTRACT

Compounds that inhibit HIF-2α, and compositions containing the compound(s) and methods for synthesizing the compounds, are described herein. Also described are the use of such compounds and compositions for the treatment of a diverse array of diseases, disorders, and conditions, including cancer- and immune-related disorders that are mediated, at least in part, by HIF-2α.

### CROSS-REFERENCES TO RELATED APPLICATIONS

This application claims the benefit of priority under 35 U.S.C. § 119(e) to U.S. Provisional Application No. 62/943,632, filed Dec. 4, 2019, which is hereby incorporated by reference in its entirety for all purposes.

### BACKGROUND OF THE INVENTION

Hypoxia-inducible factor (HIF) transcription factors play an integral role in cellular response to low oxygen availability. [Immunity. 2014 Oct. 16; 41(4): 518-528.] HIFs are heterodimeric transcription factors consisting of a common constitutive subunit called the aryl hydrocarbon receptor nuclear translocator (ARNT, or HIF-β) and one of three HIF-α subunits. [J. Med. Chem. 2015, 58, 5930-5941.] Under normal conditions, the α-subunits are hydroxylated at conserved proline residues by prolyl-4-hydroxylases (PHDs), and subsequently targeted for degredation by the von Hippel-Lindau (pVHL) ubiquitin E3 ligase complex. [Cancer Res 2006; 66(12): 6264-70] However, under hypoxic conditions, HIF-α accumlate and enter the nucleus to activate the expression of genes that regulate metabolism, angiogenesis, cell proliferation and survival, immune evasion, and inflammatory response. [J. Med. Chem. 2018, 61, 9691-9721.]

Of the three different α-subunit isoforms, HIF-1α, HIF-2α and the less characterized HIF-3α, HIF-1α and HIF-2α overexpression have been associated with poor clinical outcomes in patients with various cancers. Specifically, HIF-2α has been found to be a marker of poor prognosis in glioblastoma, neuroblastoma, head and neck squamous carcinoma, and non-smalll cell lung cancer. Hypoxia is also prevalent in many acute and chronic inflammatory disorders, such as inflammatory bowel disease and rheumatoid arthritis. [J. Clin Invest. 2016; 126(10):3661-3671.]

In view of the significant role of HIF-2α in cancer, inflammation and other disorders, there is a need in the art for HIF-2α inhibitors. The present invention addresses this need and provides related advantages as well.

### BRIEF SUMMARY OF THE INVENTION

The present invention relates to compounds that inhibit the activity of hypoxia-inducible factor (HIF) family of transcription factors, particularly HIF-2a. The compounds are represented by Formula (I):

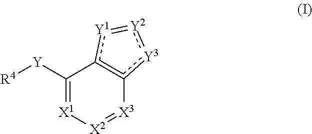

or a pharmaceutically acceptable salt, hydrate, or solvate thereof, wherein X¹, X², X³, Y, Y¹, Y², Y³, R⁴ and the dashed bonds have the meanings defined herein below.

In a related aspect, provided herein are methods for treating a disease or disorder mediated by HIF-2α in a subject (e.g., a human) comprising administering to the subject a therapeutically effective amount of at least one HIF-2α inhibitor described herein. Diseases and disorders mediated by HIF-2α include cancer, inflammation, autoimmune disorders and metabolic disorders, as described hereafter. Other diseases, disorders and conditions that can be treated or prevented, in whole or in part, by modulation of HIF-2α activity are candidate indications for the HIF-2α inhibitor compounds provided herein.

Also provided herein is the use of the described HIF-2α inhibitors in combination with one or more additional agents as hereinafter described.

### DETAILED DESCRIPTION OF THE INVENTION

Before the present invention is further described, it is to be understood that the invention is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

As used herein, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Definitions

Unless otherwise indicated, the following terms are intended to have the meaning set forth below. Other terms are defined elsewhere throughout the specification.

The term “alkyl”, by itself or as part of another substituent, means, unless otherwise stated, a straight or branched chain hydrocarbon radical, having the number of carbon atoms designated (i.e. C₁₋₈ means one to eight carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups, in some embodiments, can be substituted or unsubstituted. When a group comprising an alkylene is optionally substituted, it is understood that the optional substitutions may be on the alkylene portion of the moiety.

The term “cycloalkyl” refers to hydrocarbon rings having the indicated number of ring atoms (e.g., C₃₋₆ cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. In some embodiments, the cycloalkyl compounds of the present disclosure are monocyclic C₃₋₆ cycloalkyl moieties.

The term “heterocycloalkyl” refers to a cycloalkyl ring having the indicated number of ring vertices (or members) and having from one to five heteroatoms selected from N, O, and S, which replace one to five of the carbon vertices, and wherein the nitrogen and sulfur atoms are optionally oxidized, and the nitrogen atom(s) are optionally quaternized. The heterocycloalkyl may be a monocyclic, a bicyclic or a polycylic ring system, and may have one or two double bonds connecting ring vertices. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom.

As used herein, a wavy line, “ [symbol] ”, that intersects a single, double or triple bond in any chemical structure depicted herein, represent the point attachment of the single, double, or triple bond to the remainder of the molecule. Additionally, a bond extending to the center of a ring (e.g., a phenyl ring) is meant to indicate attachment at any of the available ring vertices. One of skill in the art will understand that multiple substituents shown as being attached to a ring will occupy ring vertices that provide stable compounds and are otherwise sterically compatible. For a divalent component, a representation is meant to include either orientation (forward or reverse). For example, the group “—C(O)NH—” is meant to include a linkage in either orientation: —C(O)NH— or —NHC(O)—, and similarly, “—O—CH₂CH₂—” is meant to include both —O—CH₂CH₂— and —CH₂CH₂—O—.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. Non-limiting examples of aryl groups include phenyl, naphthyl and biphenyl. The term is also meant to include fused cycloalkylphenyl and heterocycloalkylphenyl ring systems such as, for example, indane, tetrahydronaphthalene, chromane and isochromane rings. As a substituent group, the point of attachment to the remainder of the molecule, for a fused ring system can be through a carbon atom on the aromatic portion, a carbon atom on the cycloalkyl portion, or an atom on the heterocycloalkyl portion.

The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for a heteroaryl ring can be selected from the group of acceptable substituents described below.

The above terms (e.g., “alkyl,” “aryl” and “heteroaryl”), in some embodiments, will be optionally substituted. Selected substituents for each type of radical are provided below.

Optional substituents for the alkyl radicals (including those groups often referred to as alkylene, alkenyl, and alkynyl) can be a variety of groups selected from:

halogen, —OR′, —NR′R″, —SR′, —SiR′R″R′″, —OC(O)R′, —C(O)R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —CN (cyano), —NO₂, aryl, aryloxy, oxo, cycloalkyl and heterocycloalkyl in a number ranging from zero to (2 m′+1), where m′ is the total number of carbon atoms in such radical. R′, R″ and R′″ each independently refer to hydrogen, unsubstituted C₁₋₈ alkyl, unsubstituted aryl, aryl substituted with 1-3 halogens, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl.

Optional substituents for the cycloalkyl and heterocycloalkyl radicals can be a variety of groups selected from: alkyl optionally substituted with C(O)OR′, halogen, —OR′, —NR′R″, —SR′, —SiR′R″R′″, —OC(O)R′, —C(O)R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —CN (cyano), —NO₂, aryl, aryloxy and oxo. R′, R″ and R′″ each independently refer to hydrogen, unsubstituted C₁₋₈ alkyl, unsubstituted aryl, aryl substituted with 1-3 halogens, C₁₋₈ alkoxy or C₁₋₈ thioalkoxy groups, or unsubstituted aryl-C₁₋₄ alkyl groups.

Similarly, optional substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —N₃, perfluoro(C₁₋₄)alkoxy, and perfluoro(C₁₋₄)alkyl, in a number ranging from zero to the total number of open valences on the aromatic ring system; and where R′, R″ and R′″ are independently selected from hydrogen, C₁₋₈ alkyl, C₁₋₈ haloalkyl, C₃₋₆ cycloalkyl, C₂₋₈ alkenyl and C₂₋₈ alkynyl. Other suitable substituents include each of the above aryl substituents attached to a ring atom by an alkylene tether of from 1-6 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2.

Alternatively, two of the substituents on adjacent atoms of the aryl or heteroaryl ring may optionally be replaced with a substituent of the formula -A-(CR^{f}R^{g})ᵣ—B—, wherein A and B are independently —CH₂—, —O—, —NH—, —S—, —S(O)—, —S(O)₂—, —S(O)₂NR′— or a single bond, r is an integer of from 1 to 3, and R^{f} and R^{g} are each independently H or halogen. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention may be present, under particular conditions, as polymorphs. Polymorphism refers to the ability of a solid material to exist in more than one crystal structure form or phase, wherein the molecules in the crystal lattice have different arrangements or conformations. If such types of differences exist due to packing it is referred to as “packing polymorphism”, and if they exist due to differences in conformation it is referred to as “conformational polymorphism”. Different polymorphs of the same compound often display different physical properties, including packing properties, spectroscopic properties, thermodynamic properties, solubility, and melting point; kinetic properties such as rate of dissolution and stability; and mechanical properties such as hardness and tensile strength.

Polymorphs can be classified as one of two types according to their stability with respect to different ranges of temperature and pressure. In a monotropic system, only one polymorph (i.e., monotrope) is stable, and it exhibits lower free energy content and solubility at all temperatures and pressure below melting point. In an enantiotropic system, one polymorph is stable at a certain temperature and pressure, while the other polymorph(s) is stable at various temperatures and pressure.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention.

The compounds of the present invention may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, an inhibitor of HIF-2α, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering an inhibitor of HIF-2α or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering an HIF-2α inhibitor or a pharmaceutical composition comprising the same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a HIF-2α inhibitor (or, e.g., a metabolite thereof) at a particular time post-administration may be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The term “small molecules” refers to chemical compounds having a molecular weight that is less than about 10 kDa, less than about 2 kDa, or less than about 1kDa. Small molecules include, but are not limited to, inorganic molecules, organic molecules, organic molecules containing an inorganic component, molecules comprising a radioactive atom, and synthetic molecules. Therapeutically, a small molecule may be more permeable to cells, less susceptible to degradation, and less likely to elicit an immune response than large molecules.

The terms “inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of HIF-2α, either directly or indirectly. A modulator may act alone, or it may use a cofactor, e.g., a protein, metal ion, or small molecule. Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently (though not always) means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity.

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

Compounds that are selective may be particularly useful in the treatment of certain disorders or may offer a reduced likelihood of undesired side effects. In one embodiment, compounds of the present disclosure are selective over other HIF isoforms. In still another embodiment, the compounds of the present disclosure are selective over other kinases and targets in the HIF signaling pathway. Specific examples include HIF-1α and cytochrome P450 enzymes. Selectivity may be determined, for example, by comparing the inhibition of a compound as described herein against HIF-2α against the inhibition of a compound as described herein against another protein or isoform. In one embodiment, the selective inhibition of HIF-2a is at least 1000 times greater, 500 times greater, or 100 times greater, or 20 times greater than inhibition of another protein or isoform.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

### Compounds of the Invention

In one particular aspect, provided herein are compounds having Formula (I):

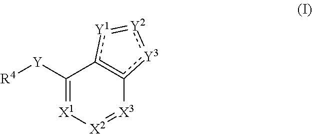

or a pharmaceutically acceptable salt, hydrate, or solvate thereof, wherein, the dashed bonds are single or double bonds consistent with the groups provided for Y¹, Y² and Y³;
X¹ is CR¹ or N;
X² is CR² or N;
X³ is CR³ or N;
Y is selected from the group consisting of —O—, —C(R^{a})(R^{b})—, —N(R^{a})—, —C(R^{a})(R^{b})—N(R^{a})—, —S— and —S(O)₂—;
Y¹, Y² and Y³ are each independently selected from the group consisting of CR⁵, NR⁶ and N, wherein one of Y¹, Y² and Y³ is N, and one of Y¹, Y² and Y³ is NR⁶;
R¹ and R² are each members independently selected from the group consisting of H, halogen, CN, —NO₂, C₁₋₄ alkyl, C₁₋₄ haloalkyl and C₁₋₄ haloalkoxy;
R³ is a member selected from the group consisting of H, halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, C₁₋₄ haloalkoxy, C₆₋₁₀ aryl and 5-10 membered heteroaryl having 1 to 4 heteroatom ring vertices independently selected from the group consisting of N, O, and S;
when R¹, R² and R³ are each present, at least one is other than H;
R⁴ is a member selected from the group consisting of C₁₋₈ alkyl, C₁₋₈ alkoxy, C₃₋₈ cycloalkyl, C₆₋₁₀ aryl, and 6-membered heteroaryl having 1 to 4 heteroatom ring vertices independently selected from the group consisting of N, O, and S;
each R⁵ is a member selected from the group consisting of H, —NO₂, —S(O)₂R^{a}, —S(O)₂NR^{a}R^{b}, —S(O)(NH)R^{a}, —C(O)R^{a}, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S;
each R⁶ is a member selected from the group consisting of H, C₁₋₈ alkyl, C₆₋₁₀ aryl and 5-10 membered heteroaryl having 1 to 4 heteroatom ring vertices independently selected from the group consisting of N, O, and S;
wherein each R^{a} and R^{b} is independently selected from the group consisting of H, C₁₋₈ alkyl, C₁₋₈ alkoxy, C₁₋₈ haloalkyl, C₁₋₈ haloalkoxy, and C₁₋₈ hydroxyalkyl, provided that when combined with the groups to which R^{a} and R^{b} are attached, N-oxide and peroxide linkages are not formed;
and for each R⁴, R⁵ and R⁶, each C₃₋₈ cycloalkyl, C₆₋₁₀ aryl and heteroaryl is unsubstituted or substituted with from one to five R^{c};
wherein each R^{c} is independently selected from the group consisting of halogen, CN, —NO₂, C₁₋₈ alkyl, C₁₋₈ alkoxy, C₁₋₈ haloalkyl, —S(O)₂R^{d}, —C(O)NR^{d}R^{e} and —P(O)R^{d}R^{e};
and R^{d} and R^{e} are each independently selected from the group consisting of H, C₁₋₈ alkyl, C₁₋₈ alkoxy, C₁₋₈ haloalkyl and C₁₋₈ haloalkoxy.

In some selected embodiments, the compound of Formula (I) or a pharmaceutically acceptable salt, hydrate, or solvate thereof, is a compound wherein Y is —O—.

In some selected embodiments, the compound of Formula (I) or a pharmaceutically acceptable salt, hydrate, or solvate thereof, is a compound wherein Y is —O—; and Y¹ is CR⁵.

In some selected embodiments, the compound of Formula (I) or a pharmaceutically acceptable salt, hydrate, or solvate thereof, is a compound wherein Y is —O—; Y¹ is CR⁵; and Y² is N.

In some selected embodiments, the compound of Formula (I) or a pharmaceutically acceptable salt, hydrate, or solvate thereof, is a compound wherein Y is —O—; Y¹ is CR⁵; Y² is N; and Y³ is NH.

In some selected embodiments, the compound of Formula (I) or a pharmaceutically acceptable salt, hydrate, or solvate thereof, is a compound wherein Y is —O—; Y¹ is NH; Y² is N; and Y³ is CR⁵.

In some selected embodiments, the compound of Formula (I) or a pharmaceutically acceptable salt, hydrate, or solvate thereof, is a compound wherein Y¹ is CR⁵; Y² is N; Y³ is NH; R³ is other than H; and each R⁵ is a member selected from the group consisting

of —S(O)₂R^{a}, —S(O)₂NR^{a}R^{b}, —S(O)(NH)R^{a}, —C(O)R^{a}, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S.

In some selected embodiments, the compound of Formula (I) is represented by Formula (I-ai):

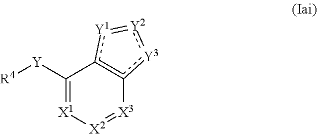

wherein, the dashed bonds are single or double bonds consistent with the groups provided for Y¹, Y² and Y³;
X¹ is CR¹ or N;
X² is CR² or N;
X³ is CR³ or N;
Y is selected from the group consisting of —O—, —C(R^{a})(R^{b})—, —N(R^{a})—, and —C(R^{a})(R^{b})—N(R^{a})—;
Y¹, Y² and Y³ are each independently selected from the group consisting of CR⁵, NR⁶ and N, wherein one of Y¹, Y² and Y³ is N, and one of Y¹, Y² and Y³ is NR⁶;
R¹ and R² are each members independently selected from the group consisting of H, halogen, and CN;
R³ is a member selected from the group consisting of H, halogen, CN, —S(O)₂R^{a}, and C₁ haloalkoxy;
when R¹, R² and R³ are each present, at least one is other than H;
R⁴ is a member selected from the group consisting of C₃₋₅ cycloalkyl, C₆ aryl, and 6-membered heteroaryl having 1-3 heteroatoms selected from 0 and N, wherein each of C₃₋₅ cycloalkyl, C₆ aryl, and 6-membered heteroaryl is substituted or unsubstituted with 1-3 R^{c}
each R⁵ is a member selected from the group consisting of H, CN, halogen, C₁₋₃ alkyl, C₁₋₃ alkoxy, C₁₋₃ alkoxymethyl, C₁₋₃ haloalkyl,
each R⁶ is a member selected from the group consisting of H and C₁₋₃ alkyl;
wherein each R^{a} and R^{b} is independently selected from the group consisting of H, C₁₋₃ alkyl, C₁₋₃ alkoxy, C₁₋₃ haloalkyl, C₁₋₃ haloalkoxy, and C₁₋₃ hydroxyalkyl; wherein each R^{c} is independently selected from the group consisting of F, Cl, CN, CH₃

In some selected embodiments, the compound of Formula (I) is represented by Formula (I-b):

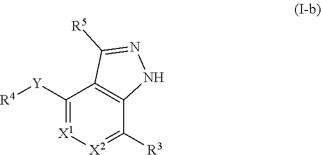

or a pharmaceutically acceptable salt, hydrate, or solvate thereof, wherein
Y is selected from the group consisting of a bond, —O—, —C(R^{a})(R^{b})—, —N(R^{a})—, —C(R^{a})(R^{b})—N(R^{a})—, —S— and —S(O)₂—;
X¹ is CR¹ or N;
X² is CR² or N;
R¹ and R² are each members independently selected from the group consisting of H, halogen, CN, —NO₂ and C₁₋₄ haloalkyl;
R³ is a member selected from the group consisting of H, —NO₂, —S(O)₂R^{a}, —C(O)NR^{a}R^{b}, CN, halogen, —P(O)R^{a}R^{b}, C₁₋₈ alkyl, C₁₋₈ alkoxy, C₁₋₈ haloalkyl, C₆₋₁₀ aryl and 5-10 membered heteroaryl;
when R¹, R² and R³ are each present, at least one is other than H;
R⁴ is a member selected from the group consisting of C₁₋₈ alkyl, C₁₋₈ alkoxy, C₃₋₈ cycloalkyl, C₆₋₁₀ aryl, 6-membered heteroaryl having 1 to 4 heteroatom ring vertices independently selected from the group consisting of N, O, and S;
each R⁵ is a member selected from the group consisting of —NO₂, —S(O)₂R^{a}, —S(O)₂NR^{a}R^{b}, —S(O)(NH)R^{a}, —C(O)R^{a}, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S;
wherein each R^{a} and R^{b} is independently selected from the group consisting of H, C₁₋₈ alkyl, C₁₋₈ alkoxy, C₁₋₈ haloalkyl, C₁₋₈ haloalkoxy, and C₁₋₈ hydroxyalkyl;
and each C₃₋₈ cycloalkyl, C₆₋₁₀ aryl and heteroaryl is unsubstituted or substituted with from one to five R^{e};
wherein each R^{c} is independently selected from the group consisting of halogen, CN, —NO₂, C₁₋₈ alkyl, C₁₋₈ alkoxy, C₁₋₈ haloalkyl, —S(O)₂R^{d}, —C(O)NR^{d}R^{e} and —P(O)R^{d}R^{e};
and R^{d} and R^{e} are each independently selected from the group consisting of H, C₁₋₈ alkyl, C₁₋₈ alkoxy, C₁₋₈ haloalkyl and C₁₋₈ haloalkoxy.

In some embodiments, the compound of Formula (I-b) is a compound or a pharmaceutically acceptable salt, hydrate, or solvate thereof wherein R⁴ is selected from the group consisting of phenyl, pyridyl, pyrimidinyl, pyrazinyl, 1,2,4-triazinyl and 1,3,5-triazinyl, each of which is unsubstituted or substituted with from 1 to 3 independently selected R^{c} groups.

In some selected embodiments, the compound of Formula (I) is represented by Formula (I-c):

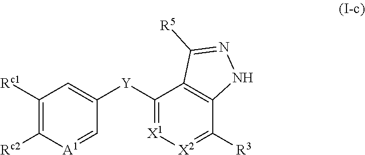

or a pharmaceutically acceptable salt, hydrate, or solvate thereof, wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH;
R^{c1}, R^{c2} and R^{c3} are each independently selected from the group consisting of H, F, Cl, CN, CF₃, OCF₃ and C₁₋₄ alkyl.

In some selected embodiments, the compound of Formula (I) is represented by Formula (I-d):

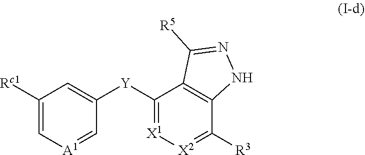

or a pharmaceutically acceptable salt, hydrate, or solvate thereof, wherein
A¹ is N or CR^{c3};
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} is selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₄ alkyl; and
the remaining groups have the meanings provided for Formula (I).

In some selected embodiments, the compound of Formula (I) is represented by Formula (I-e):

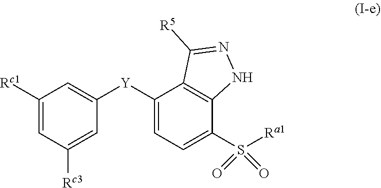

or a pharmaceutically acceptable salt, hydrate, or solvate thereof, wherein
Y is —O— or —NH—;
R⁵ is selected from the group consisting of H, F, Cl, CN, I, CF₃ and CH₂OH;
R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-f):

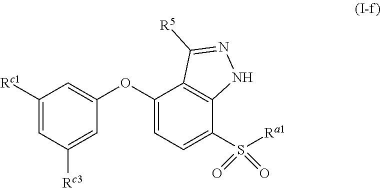

or a pharmaceutically acceptable salt, hydrate, or solvate thereof, wherein

R⁵ is selected from the group consisting of H, F, Cl, CN, I, CF₃ and CH₂OH;

R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-g):

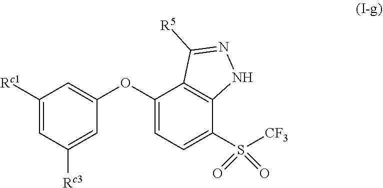

or a pharmaceutically acceptable salt, hydrate, or solvate thereof, wherein
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-h):

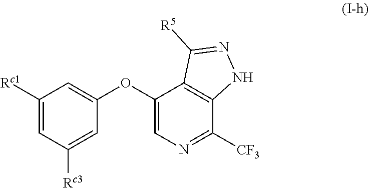

or a pharmaceutically acceptable salt, hydrate, or solvate thereof, wherein
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-i):

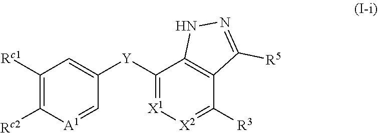

or a pharmaceutically acceptable salt, hydrate, or solvate thereof, wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH;
R^{c1}, R^{c2} and R^{c3} are each independently selected from the group consisting of H, F, Cl, CN, CF₃, OCF₃ and C₁₋₄ alkyl; and
the remaining groups have the meanings provided for Formula (I).

In some selected embodiments, the compound of Formula (I) is represented by Formula (I-j):

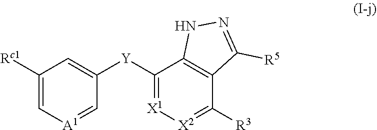

or a pharmaceutically acceptable salt, hydrate, or solvate thereof, wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₄ alkyl; and
the remaining groups have the meanings provided for Formula (I).

In some selected embodiments, the compound of Formula (I) is represented by Formula (I-k):

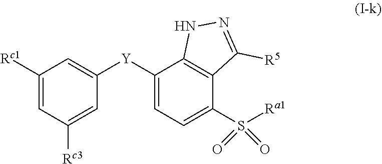

or a pharmaceutically acceptable salt, hydrate, or solvate thereof, wherein
Y is —O— or —NH—;
R⁵ is selected from the group consisting of H, F, Cl, CN, I, CF₃ and CH₂OH;
R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-l):

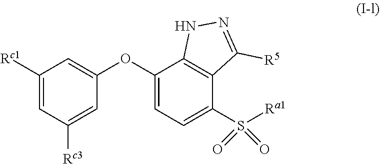

or a pharmaceutically acceptable salt, hydrate, or solvate thereof, wherein
R⁵ is selected from the group consisting of H, F, Cl, CN, I, CF₃ and CH₂OH;
R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-m):

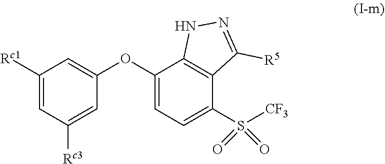

or a pharmaceutically acceptable salt, hydrate, or solvate thereof, wherein
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₆ alkyl.

In some selected embodiments, the compound of Formula (I) is represented by Formula (I-n):

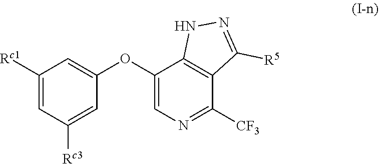

or a pharmaceutically acceptable salt, hydrate, or solvate thereof, wherein
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₆ alkyl.

In some selected embodiments, any one compound of Table 1, is provided.

Identification of HIF-2α inhibitors Possessing Desirable Characteristics

The present invention is drawn, in part, to the identification of inhibitors of HIF-2α with at least one property or characteristic that is of therapeutic relevance. Candidate inhibitors may be identified by using, for example, an art-accepted assay or model, examples of which are described herein.

After identification, candidate inhibitors can be further evaluated by using techniques that provide data regarding characteristics of the inhibitors (e.g., pharmacokinetic parameters, means of determining solubility or stability). Comparisons of the candidate inhibitors to a reference standard (which may be the “best-of-class” of current inhibitors) are indicative of the potential viability of such candidates.

### Methods of Synthesis

### General Methods for the Preparation of Compounds of the Claims

For the most efficient preparation of any particular compound of the invention, one skilled in the art will recognize that the timing and the order of connection of the fragments and modification of the functionality present in any of the fragments may vary in the preparation of any given compound. A variety of methods have been used to prepare compounds of the invention, some of which are exemplified in the examples.

Prodrugs and Other Means of Drug Delivery and/or Half-Life Extension

In some aspects of the present invention, compounds described herein are administered in prodrug form.

In order to effect extension of therapeutic activity, drug molecules may be engineered to utilize carriers for delivery. Such carriers are either used in a non-covalent fashion, with the drug moiety physicochemically formulated into a solvent-carrier mixture, or by permanent covalent attachment of a carrier reagent to one of the drug moiety's functional groups (see generally WO 2015/0202317).

Several non-covalent approaches are favored. By way of example, but not limitation, in certain embodiments depot formulations comprising non-covalent drug encapsulation into polymeric carriers are employed. In such formulations, the drug molecule is combined with carrier material and processed such that the drug molecule becomes distributed inside the bulk carrier. Examples include microparticle polymer-drug aggregates (e.g., Degradex® Microspheres (Phosphorex, Inc.)), which are administered as an injectable suspension; polymer-drug molecule aggregates formulated as gels (e.g., Lupron Depot® (AbbVie Inc.)), which are administered as a single bolus injection; and liposomal formulations (e.g., DepoCyt® (Pacira Pharmaceuticals)), where the carrier may be a polymeric or non-polymeric entity capable of solubilizing the drug. In these formulations, release of the drug molecule may occur when the carrier swells or physically deteriorates. In other instances, chemical degradation allows diffusion of the drug into the biological environment; such chemical degradation processes may be autohydrolytic or enzyme-catalyzed. Among other limitations, non-covalent drug encapsulation requires prevention of uncontrolled release of the drug, and dependence of the release mechanism of the drug upon biodegradation may cause interpatient variability.

In particular embodiments, drug molecules, including both small molecules and large molecules, are conjugated to a carrier through permanent covalent bonds. Certain small molecule therapeutics that exhibit low solubility in aqueous fluids may be solubilized by conjugation to hydrophilic polymers, examples of which are described elsewhere herein. Regarding large molecule proteins, half-life extension may be achieved by, for example, permanent covalent modification with a palmitoyl moiety, and by permanent covalent modification with another protein that itself has an extended half-life (e.g., Albuferon®). In general, drug molecules show decreased biological activity when a carrier is covalently conjugated to the drug.

In certain instances, limitations associated with either drug molecules comprising non-covalent polymer mixtures or permanent covalent attachment may be successfully addressed by employing a prodrug approach for chemical conjugation of the drug to the polymer carrier. In this context, therapeutic agents that are inactive or less active than the drug moiety itself are predictably transformed into active molecular entities. The reduced biological activity of the prodrug as compared to the released drug is advantageous if a slow or controlled release of the drug is desired. In such instances, release of the drug occurs over time, thereby reducing the necessity of repeated and frequent administration of the drug. A prodrug approach may also be advantageous when the drug moiety itself is not absorbed, or has less than optimal absorption, in the gastrointestinal tract; in these instances, the prodrug facilitates absorption of the drug moiety and is then cleaved off at some later time (e.g., via first-pass metabolism). The biologically active drug molecule is typically linked to the polymeric carrier moiety by a temporary bond formed between the carrier moiety and a hydroxy, amino or carboxy group of the drug molecule.

The approaches described above are associated with several limitations. Prodrug activation may occur by enzymatic or non-enzymatic cleavage of the temporary bond between the carrier and the drug molecule, or a sequential combination of both (e.g., an enzymatic step followed by a non-enzymatic modification). In an enzyme-free in vitro environment (e.g., an aqueous buffer solution), a temporary bond such as an ester or amide may undergo hydrolysis, but the corresponding rate of hydrolysis may be such that it is outside the therapeutically useful range. In contrast, in an in vivo environment, esterases or amidases are typically present, and the esterases and amidases may cause significant catalytic acceleration of the kinetics of hydrolysis from two-fold up to several orders of magnitude (see, e.g., Greenwald et al., (1999) J Med Chem 42(18):3857-67).

As described herein, prodrugs may be classified as i) bioprecursors and ii) carrier-linked prodrugs. Bioprecursors do not contain a carrier group and are activated by the metabolic creation of a functional group. In contrast, in carrier-linked prodrugs the active substance is conjugated to a carrier moiety via a temporary linkage at a functional group of the bioactive entity. Preferred functional groups are hydroxyl or amino groups. Both the attachment chemistry and hydrolysis conditions depend on the type of functional group employed. The carrier may be biologically inert (e.g., PEG) or may have targeting properties (e.g., an antibody). Cleavage of the carrier moiety of a carrier-linked prodrug results in the bioactive entity of interest, and the nature of the deprotected functional group of the bioactive entity often contributes to its bioactivity.

The patent and scientific literature describe many macromolecular prodrugs where the temporary linkage is a labile ester bond. In these cases, the functional group of the bioactive entity is either a hydroxyl group or a carboxylic acid (see, e.g. Cheng et al. (2003) Bioconjugate Chem 14:1007-17). In addition, it is often advantageous for biomacromolecules and certain small molecule drugs to link the carrier to an amino group(s) of the bioactive entity (e.g., the N-terminus or lysine amino groups of proteins). During preparation of the prodrug, the amino groups may be more chemoselectively addressed due to their greater nucleophilicity compared to hydroxylic or phenolic groups. This is especially relevant for proteins and peptides containing a great variety of different reactive functionalities, where non-selective conjugation reactions lead to undesired product mixtures requiring extensive characterization or purification, thus decreasing reaction yield and therapeutic efficiency of the active moiety.

In general, amide bonds are more stable against hydrolysis than ester bonds, and the rate of cleavage of the amide bond may be too slow for therapeutic utility in a carrier-linked prodrug. As a result, it may be advantageous to add structural chemical components in order to effect control over the cleavability of the prodrug amide bond. These additional cleavage-controlling chemical components that are provided neither by the carrier entity nor by the drug are generally referred to as “linkers”. Prodrug linkers can have a major effect on the rate of hydrolysis of temporary bond, and variation of the chemical nature of the linkers often results in particular properties. Prodrug activation of amine-containing biologically active moieties by specific enzymes for targeted release requires that the structure of the linker display a structural motif recognized as a substrate by a corresponding endogenous enzyme. In these cases, the cleavage of the temporary bond occurs in a one-step process which is catalyzed by the enzyme. For example, the enzymatic release of cytarabin is effected by the protease plasmin, which concentration is relatively high in various kinds of tumor mass.

Interpatient variability is a major drawback of predominant enzymatic cleavage. Enzyme levels may differ significantly between subjects resulting in biological variation of prodrug activation by the enzymatic cleavage. Enzyme levels may also vary depending on the site of administration (e.g., for subcutaneous injection, certain areas of the body yield more predictable therapeutic effects than others). In addition, it is difficult to establish an in vivo—in vitro correlation of the pharmacokinetic properties for enzyme-dependent carrier-linked prodrugs.

Other carrier prodrugs employing temporary linkages to amino groups in the drug moiety are based on a cascade mechanism. Cascade cleavage is enabled by linker compounds that are composed of a structural combination of a masking group and an activating group. The masking group is attached to the activating group by means of a first temporary linkage such as an ester or a carbamate. The activating group is attached to an amino group of the drug molecule through a second temporary linkage (e.g., a carbamate). The stability or susceptibility to hydrolysis of the second temporary linkage is dependent on the presence or absence of the masking group. In the presence of the masking group, the second temporary linkage is highly stable and unlikely to release the drug molecule with therapeutically useful kinetics, whereas in the absence of the masking group this linkage becomes highly labile, resulting in rapid cleavage and release of the drug moiety.

The cleavage of the first temporary linkage is the rate-limiting step in the cascade mechanism. The first step may induce a molecular rearrangement of the activating group (e.g., a 1,6-elimination as described in Greenwald et al. (1999) J Med Chem 42:3657-67), and the rearrangement renders the second temporary linkage much more labile such that its cleavage is induced. Ideally, the cleavage rate of the first temporary linkage is identical to the desired release rate for the drug molecule in a given therapeutic scenario. In addition, it is desirable that the cleavage of the second temporary linkage be substantially instantaneous after its lability has been induced by cleavage of the first temporary bond.

Another embodiment comprises polymeric amino-containing prodrugs based on trimethyl lock lactonization (see, e.g., Greenwald et al. (2000) J Med Chem 43(3):457-87). In this prodrug system, substituted o-hydroxyphenyl-dimethylpropionic acid is linked to PEG by an ester, carbonate, or carbamate group as a first temporary linkage and to an amino group of a drug molecule by means of an amide bond as a second temporary linkage. The rate-determining step in drug release is the enzymatic cleavage of the first linkage, which is followed by fast amide cleavage by lactonization, releasing an aromatic lactone side product. The primary disadvantage of the prodrug systems described by Greenwald et al. is the release of highly reactive and potentially toxic aromatic small molecule side products like quinone methides or aromatic lactones after cleavage of the temporary linkage. The potentially toxic entities are released in a 1:1 stoichiometry with the drug and can assume high in vivo concentrations.

In certain embodiments of cascade prodrugs comprising aromatic activating groups based on 1,6-elimination, the masking group is structurally separate from the carrier. This may be effected by employing a stable bond between the polymer carrier and the activating group, wherein the stable bond does not participate in the cascade cleavage mechanism. If the carrier is not serving as a masking group and the activating group is coupled to the carrier by means of a stable bond, release of potentially toxic side products (such as the activating group) is avoided. The stable attachment of the activating group and the polymer also suppresses the release of drug-linker intermediates with undefined pharmacology.

A first example of the approach described in the preceding paragraph comprises a polymeric prodrug system based on a mandelic acid activating group (see, e.g., Shabat et al. (2004) Chem Eur J 10:2626-34). In this approach the masking group is linked to the activating group by a carbamate bond. The activating group is conjugated permanently to a polyacrylamide polymer via an amide bond. After enzymatic activation of the masking group by a catalytic antibody, the masking group is cleaved by cyclization and the drug is released; the activating group is still connected to the polyacrylamide polymer after drug release. A similar prodrug system is based on a mandelic acid activating group and an enzymatically cleavable ester-linked masking group (see, e.g., Lee et al. (2004) Angew Chem 116:1707-10).

When the aforementioned linkers are used, the 1,6-elimination step still generates a highly reactive aromatic intermediate. Even if the aromatic moiety remains permanently attached to the polymeric carrier, side reactions with potentially toxic by-products or immunogenic effects may result. Thus, it is advantageous to generate linker technologies for forming polymeric prodrugs of amine-containing active agents using aliphatic prodrug linkers that are not enzyme-dependent and do not generate reactive aromatic intermediates during cleavage. One such example uses PEG5000-maleic anhydride for the reversible modification of amino groups in tissue-type plasminogen activator and urokinase (see, e.g. (1987) Garman et al. FEBS Lett 223(2):361-65). Regeneration of functional enzyme from PEG-uPA conjugate upon incubation at pH 7.4 buffer by cleavage of the maleamic acid linkage follows first order kinetics with a half-life of roughly 6 hours. A disadvantage of the maleamic acid linkage is the lack of stability of the conjugate at lower pH values.

A further approach comprises a PEG cascade prodrug system based on N,N-bis-(2-hydroxyethyl)glycine amide (bicine) linker (see e.g. (2004) J Med Chem 47:726-34). In this system, two PEG carrier molecules are linked via temporary bonds to a bicine molecule coupled to an amino group of the drug molecule. The first steps in prodrug activation involves the enzymatic cleavage of the first temporary linkages connecting both PEG carrier molecules with the hydroxy groups of the bicine activating group. Different linkages between PEG and bicine result in different prodrug activation kinetics. The second step in prodrug activation involves the cleavage of the second temporary linkage connecting the bicine activating group to the amino group of the drug molecule. A disadvantage of this system is the slow hydrolysis rate of this second temporary bicine amide linkage, which results in the release of a bicine-modified prodrug intermediate that may show different pharmacokinetic, immunogenic, toxicity and pharmacodynamic properties as compared to the native parent drug molecule.

In particular embodiments, dipeptides are utilized for prodrug development for targeting or targeted transport as they are substrates for enzymes or biotransport systems. The non-enzymatic route for dipeptide prodrug formation, that is, the ability to undergo intramolecular cyclization to form the corresponding diketopiperazine (DKP) and release the active drug, is not well defined.

In some embodiments, dipeptides are attached to a drug moiety via ester bonds, as was described for dipeptide esters of the drug paracetamol (Gomes et al. (2005) Bio & Med Chem Lett). In this case, the cyclization reaction consists of a nucleophilic attack of the N-terminal amine of the peptide on the ester carbon atom to form a tetrahedral intermediate, which is followed by a proton transfer from the amine to the leaving group oxyanion with simultaneous formation of a peptide bond to give the cyclic DKP product and free drug. This method is applicable to hydroxyl-containing drugs in vitro but has been found to compete with enzymatic hydrolysis of the ester bond in vivo, as corresponding dipeptide esters released paracetamol at a much faster rate than in buffer (Gomes et al. (Molecules 12 (2007) 2484-2506). Susceptibility of dipeptide-based prodrugs to peptidases may be addressed by incorporating at least one non-natural amino acid in the dipeptide motif. However, endogenous enzymes capable of cleaving ester bonds are not limited to peptidases, and the enzyme-dependence of such prodrug cleavage still gives rise to unpredictable in vivo performance.

In some embodiments, enzyme-dependence is intentionally engineered into DKP prodrugs, such as where dipeptide ester prodrugs are formylated at the amino terminus of the dipeptide, and enzymatic deformylation is used to initiate diketopiperazine formation and subsequent cleavage of the ester-dipeptide bond, followed by release of the drug molecule (see, e.g., U.S. Pat. No. 7,163,923). By way of further example, an octapeptide is attached by an ester linkage to the 4-hydroxyl group of vinblastine and undergoes ester bond cleavage by DKP formation after specific enzymatic removal of the N-terminal hexapeptide (see Brady et al. (2002) J Med Chem 45:4706-15).

The scope of the DKP formation reaction has also been extended to amide prodrugs. By way of example, U.S. Pat. No. 5,952,294 describes prodrug activation using diketopiperazine formation for dipeptidyl amide prodrugs of cytarabine. In this case, the temporary linkage is formed between the carbonyl of a dipeptide and the aromatic amino group of cytarabine. However, it is unlikely that a slow-release effect can be achieved for such conjugates as there is no carrier or other half-life extending moiety or functionality present.

Dipeptide prodrugs comprising bioactive peptides such as GLP-1 capable of releasing the peptide through diketopiperazine formation of the dipeptidic extension have also been described (see, e.g., WO 2009/099763). The bioactive peptide moiety may include an additional PEG chain on one of its amino acid side chain residues to achieve extended circulation of the bioactive peptide. However, this approach is associated with several significant disadvantages. First, the PEG chain has to be linked to the peptide without compromising its bioactivity, which can be difficult to achieve for many peptide-based bioactive agents. Second, as the pegylated peptide itself is bioactive, the dipeptidic promoiety has an effect on the peptide's bioactivity and may negatively affect its receptor binding properties.

Specific exemplary technologies that may be used with the compounds of the present invention include those developed by ProLynx (San Francisco, Calif.) and Ascendis Pharma (Palo Alto, Calif.). The ProLynx technology platform utilizes sets of novel linkers that are pre-programmed to cleave at different rates to allow the controlled, predictable and sustained release of small molecules and peptides from circulating semi-solid macromolecular conjugates. The technology allows for maintenance of desired steady-state serum levels of therapeutic agents for weeks to months.

The Ascendis technology platform combines the benefits of prodrug and sustained release technologies to enhance the properties of small molecules and peptides. While in circulation, proprietary prodrugs release the unmodified active parent therapeutic agent at predetermined rates governed by physiological pH and temperature conditions. Because the therapeutic agent is released in its unmodified form, it retains its original mechanism of action.

### Modifications to Enhance Inhibitor Characteristics

It is frequently beneficial, and sometimes imperative, to improve one or more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity.

Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9):3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

Other known modifications include deuteration to improve pharmacokinetics, pharmacodynamics and toxicity profiles. Due to the greater atomic mass of deuterium, cleavage of the carbon-deuterium bond requires more energy than the carbon-hydrogen bond. Because these stronger bonds are more difficult to break, the rate of drug metabolism is slower as compared to non-deuterated forms, which allows for less frequent dosing and may further reduce toxicities. (Charles Schmidt, Nature Biotechnology, 2017, 35(6): 493-494; Harbeson, S. and Tung, R., Medchem News, 2014(2): 8-22).

### Therapeutic and Prophylactic Uses

The present invention contemplates the use of the HIF-2α inhibitors described herein in the treatment or prevention of a broad range of diseases, disorders and/or conditions, and/or the symptoms thereof. While particular uses are described in detail hereafter, it is to be understood that the present invention is not so limited. Furthermore, although general categories of particular diseases, disorders and conditions are set forth hereafter, some of the diseases, disorders and conditions may be a member of more than one category, and others may not be a member of any of the disclosed categories.

In some embodiments, the HIF-2α inhibitors described herein are administered in an amount effective to reverse, stop or slow the progression of HIF-2α-mediated dysregulation.

Oncology-related Disorders. The HIF-2α inhibitors described herein can be used to treat or prevent a proliferative condition or disorder, including a cancer, for example, cancer of the uterus, cervix, breast, prostate (such as metastatic castration resistant prostate cancer), testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present invention also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. In particular embodiments, the tumor or cancer is colon cancer, ovarian cancer, breast cancer, melanoma, lung cancer, glioblastoma, or leukemia. The use of the term(s) cancer-related diseases, disorders and conditions is meant to refer broadly to conditions that are associated, directly or indirectly, with cancer, and includes, e.g., angiogenesis and precancerous conditions such as dysplasia.

In certain embodiments, a cancer may be metastatic or at risk of becoming metastatic, or may occur in a diffuse tissue, including cancers of the blood or bone marrow (e.g., leukemia).

In some embodiments, the present invention provides methods for treating a proliferative condition, cancer, tumor, or precancerous condition with a HIF-2α inhibitor and at least one additional therapeutic or diagnostic agent, examples of which are set forth elsewhere herein.

The methods of treating cancer described herein may be suitable as a first line therapy, a second line therapy, or a third line therapy.

In some embodiments, the disease or disorder is VHL-associated, for example VHL-associated renal cell carcinoma.

In one embodiment, the compounds described herein may be useful in treatment of iron overload disorders. The iron overload disorder may be primary or secondary. In one embodiment, the iron overload disorder may be hemochromatosis. In other embodiments, the compounds described herein may be useful in treating polycythemia such as, for example, polycythemia vera. In another embodiment, the compounds described herein may be useful in treating Pacak-Zhuang Syndrome. In still another embodiment, the compounds described herein may be useful for treating erythrocytosis.

Immune- and Inflammatory-related Disorders. A non-limiting list of immune- and inflammatory-related diseases, disorders and conditions which may be treated or prevented with the compounds and compositions of the present invention include arthritis (e.g., rheumatoid arthritis), kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (e.g., Crohn's disease and ulcerative colitis), chronic obstructive pulmonary disease (COPD), atherosclerosis, allergic contact dermatitis and other eczemas, systemic sclerosis, transplantation and multiple sclerosis.

In particular embodiments of the present disclosure, the HIF-2α inhibitors are used to increase or enhance an immune response to an antigen by providing adjuvant activity. In a particular embodiment, at least one antigen or vaccine is administered to a subject in combination with at least one HIF-2α inhibitor of the present invention to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one HIF-2α inhibitor of the present invention.

In some embodiments, a HIF-2α inhibitor as described herein can be combined with an immunosuppressive agent to reduce the number of immune effector cells.

Other Disorders. Embodiments of the present invention contemplate the administration of the HIF-2α inhibitors described herein to a subject for the treatment or prevention of any other disorder that may benefit from at least some level of HIF-2α inhibition. Such diseases, disorders and conditions include, for example, cardiovascular (e.g., cardiac ischemia) and metabolic (e.g., diabetes, insulin resistance, obesity) disorders.

### Pharmaceutical Compositions

The HIF-2α inhibitors of the present invention may be in the form of compositions suitable for administration to a subject. In general, such compositions are “pharmaceutical compositions” comprising an HIF-2α inhibitor(s) and one or more pharmaceutically acceptable or physiologically acceptable diluents, carriers or excipients. In certain embodiments, the HIF-2a inhibitors are present in a therapeutically acceptable amount. The pharmaceutical compositions may be used in the methods of the present invention; thus, for example, the pharmaceutical compositions can be administered ex vivo or in vivo to a subject in order to practice the therapeutic and prophylactic methods and uses described herein.

The pharmaceutical compositions of the present invention can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present invention.

The pharmaceutical compositions containing the active ingredient (e.g., an inhibitor of HIF-2α function) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a HIF-2α inhibitor contemplated by the present invention and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a HIF-2α inhibitor, including implants (e.g., implantable pumps) and catheter systems, slow injection pumps and devices, all of which are well known to the skilled artisan.

Depot injections, which are generally administered subcutaneously or intramuscularly, may also be utilized to release the HIF-2α inhibitors disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The present invention contemplates the administration of the HIF-2α inhibitors in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The HIF-2α inhibitors contemplated by the present invention may be in the form of any other suitable pharmaceutical composition (e.g., sprays for nasal or inhalation use) currently known or developed in the future.

### Routes of Administration

The present invention contemplates the administration of HIF-2α inhibitors, and compositions thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the HIF-2α inhibitors disclosed herein over a defined period of time.

Particular embodiments of the present invention contemplate oral administration.

### Combination Therapy

The present invention contemplates the use of HIF-2α inhibitors alone or in combination with one or more active therapeutic agents. The additional active therapeutic agents can be small chemical molecules; macromolecules such as proteins, antibodies, peptibodies, peptides, DNA, RNA or fragments of such macromolecules; or cellular or gene therapies. The combination therapy may target different, but complementary mechanisms of action and thereby have a synergistic therapeutic or prophylactic effect on the underlying disease, disorder, or condition. In addition or alternatively, the combination therapy may allow for a dose reduction of one or more of the agents, thereby ameliorating, reducing or eliminating adverse effects associated with one or more of the agents.

The active therapeutic agents in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In some embodiments, the additional therapeutic agent is an immunomodulatory agent. Suitable immunomodulatory agents that may be used in the present invention include CD40L, B7, and B7RP1; activating monoclonal antibodies (mAbs) to stimulatory receptors, such as, anti-CD40, anti-CD38, anti-ICOS, and 4-IBB ligand; dendritic cell antigen loading (in vitro or in vivo); anti-cancer vaccines such as dendritic cell cancer vaccines; cytokines/chemokines, such as, IL1, IL2, IL12, IL18, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); indoleamine 2,3-dioxygenase 1 (IDO1) inhibitors and immune-stimulatory oligonucleotides.

In certain embodiments, the present invention provides methods for tumor suppression of tumor growth comprising administration of a HIF-2α inhibitor described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Signal transduction inhibitors (STIs) of the present invention include: (i) bcr/abl kinase inhibitors (e.g., GLEEVEC®); (ii) epidermal growth factor (EGF) receptor inhibitors, including kinase inhibitors and antibodies; (iii) her-2/neu receptor inhibitors (e.g., HERCEPTIN®); (iv) inhibitors of Akt family kinases or the Akt pathway (e.g., Trop2 inhibotors orrapamycin); (v) cell cycle kinase inhibitors (e.g., flavopiridol); and (vi) phosphatidyl inositol kinase inhibitors. Agents involved in immunomodulation can also be used in combination with the HIF-2α inhibitors described herein for the suppression of tumor growth in cancer patients.

In some embodiments, the additional therapeutic agent is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, pomalidomide, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU) with or without leucovorin; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; anthracyclines; arginase inhibitors (see PCT/US2019/020507) and pharmaceutically acceptable salts, acids or derivatives of any of the above.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as abiraterone, enzalutamide, flutamide, nilutamide, bicalutamide, leuprolide, and goserelin; and pharmaceutically acceptable salts, acids or derivatives of any of the above. In certain embodiments, combination therapy comprises a chemotherapy regimen that includes one or more chemotherapeutic agents. In certain embodiments, combination therapy comprises administration of a hormone or related hormonal agent.

Additional treatment modalities that may be used in combination with a HIF-2α inhibitor include radiotherapy, a monoclonal antibody against a tumor antigen, a complex of a monoclonal antibody and toxin, a T-cell adjuvant, bone marrow transplant, or antigen presenting cells (e.g., dendritic cell therapy), including TLR agonists which are used to stimulate such antigen presenting cells.

In certain embodiments, the present invention contemplates the use of the compounds described herein in combination with adoptive cell therapy, a new and promising form of personalized immunotherapy in which immune cells with anti-tumor activity are administered to cancer patients. Adoptive cell therapy is being explored using tumor-infiltrating lymphocytes (TIL) and T cells engineered to express, for example, chimeric antigen receptors (CAR) or T cell receptors (TCR). Adoptive cell therapy generally involves collecting T cells from an individual, genetically modifying them to target a specific antigen or to enhance their anti-tumor effects, amplifying them to a sufficient number, and infusion of the genetically modified T cells into a cancer patient. T cells can be collected from the patient to whom the expanded cells are later reinfused (e.g., autologous) or can be collected from donor patients (e.g., allogeneic).

In certain embodiments, the present invention contemplates the use of the compounds described herein in combination with RNA interference-based therapies to silence gene expression. RNAi begins with the cleavage of longer double-stranded RNAs into small interfering RNAs (siRNAs). One strand of the siRNA is incorporated into a ribonucleoprotein complex known as the RNA-induced silencing complex (RISC), which is then used to identify mRNA molecules that are at least partially complementary to the incorporated siRNA strand. RISC can bind to or cleave the mRNA, both of which inhibits translation.

In certain embodiments, the present invention contemplates the use of the compounds described herein in combination with agents that modulate the level of adenosine. Such therapeutic agents may act on the ectonucleotides that catalyze the conversion of ATP to adenosince, including ectonucleoside triphosphate diphosphohydrolase 1 (ENTPD1, also known as CD39 or Cluster of Differentiation 39), which hydrolyzes ATP to ADP and ADP to AMP, and 5′-nucleotidase, ecto (NT5E or 5NT, also known as CD73 or Cluster of Differentiation 73), which converts AMP to adenosine. The enzymatic activities of CD39 and CD73 play strategic roles in calibrating the duration, magnitude, and chemical nature of purinergic signals delivered to various cells (e.g., immune cells). Alteration of these enzymatic activities can change the course or dictate the outcome of several pathophysiological events, including cancer, autoimmune diseases, infections, atherosclerosis, and ischemia-reperfusion injury, suggesting that these ecto-enzymes represent novel therapeutic targets for managing a variety of disorders. In one embodiment, the CD73 inhibitors are those described in WO2017/120508, WO2018/067424, WO2018/094148, and WO2020/046813.

Alternatively, such therapeutic agents can be adenosine 2 receptor (A₂R) antagonists. Adenosine can bind to and active four different G-protein coupled receptors: A₁R, A₂ₐR, A_{2b}R, and A₃R. The binding of adenosine to the A₂ₐR receptor, which is expressed on T cells, natural killer cells and myeloid cells such as dendritic cells, leads to increased intracellular levels of cyclic AMP and the impairment of maturation and/or activation of such cells. This process significantly impairs the activation of the immune system against cancer cells. In addition, A_{2A}R has been implicated in selectively enhancing anti-inflammatory cytokines, promoting the upregulation of PD-1 and CTLA-4, promoting the generation of LAG-3 and Foxp3+ regulatory T cells, and mediating the inhibition of regulatory T cells. PD-1, CTLA-4 and other immune checkpoints which are discussed further herein. Combining A₂R antagonists in the combinations described herein may provide at least an aditive effect in view of their differing mechanisms of actions. In one embodiment, the present invention contemplates combination with the adenosine receptor antagonists described in WO2018/136700, WO2018/204661, WO2018/213377, or WO2020/023846.

In certain embodiments, the present invention contemplates the use of the compounds described herein in combination with inhibitors of phosphatidylinositol 3-kinases (PI3Ks), particularly the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T-cell responses leading to decreased cancer development and spread. PI3Kγ inhibitors include those described in PCT/US2020/035920.

In certain embodiments, the present invention contemplates the use of the compounds described herein in combination with inhibitors of arginase, which has been shown to be either responsible for or to participate in inflammation-triggered immune dysfunction, tumor immune escape, immunosuppression and immunopathology of infectious disease. Exemplary arginase compounds can be found, for example, in PCT/US2019/020507 and WO/2020/102646.

Immune Checkpoint Inhibitors. The present invention contemplates the use of the inhibitors of HIF-2α function described herein in combination with immune checkpoint inhibitors.

The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism.

T-cells have been the major focus of efforts to therapeutically manipulate endogenous antitumor immunity because of i) their capacity for the selective recognition of peptides derived from proteins in all cellular compartments; ii) their capacity to directly recognize and kill antigen-expressing cells (by CD8+ effector T cells; also known as cytotoxic T lymphocytes (CTLs)); and iii) their ability to orchestrate diverse immune responses by CD4+ helper T cells, which integrate adaptive and innate effector mechanisms.

In the clinical setting, the blockade of immune checkpoints which results in the amplification of antigen-specific T cell responses has shown to be a promising approach in human cancer therapeutics.

T cell-mediated immunity includes multiple sequential steps, each of which is regulated by counterbalancing stimulatory and inhibitory signals in order to optimize the response. While nearly all inhibitory signals in the immune response ultimately modulate intracellular signaling pathways, many are initiated through membrane receptors, the ligands of which are either membrane-bound or soluble (cytokines). While co-stimulatory and inhibitory receptors and ligands that regulate T-cell activation are frequently not over-expressed in cancers relative to normal tissues, inhibitory ligands and receptors that regulate T cell effector functions in tissues are commonly overexpressed on tumor cells or on non-transformed cells associated with the tumor microenvironment. The functions of the soluble and membrane-bound receptor ligand immune checkpoints can be modulated using agonist antibodies (for co-stimulatory pathways) or antagonist antibodies (for inhibitory pathways). Thus, in contrast to most antibodies currently approved for cancer therapy, antibodies that block immune checkpoints do not target tumor cells directly, but rather target lymphocyte receptors or their ligands in order to enhance endogenous antitumor activity. [See Pardoll, (April 2012) Nature Rev. Cancer 12:252-64].

Examples of immune checkpoints (ligands and receptors), some of which are selectively upregulated in various types of tumor cells, that are candidates for blockade include PD-1 (programmed cell death protein 1); PD-L1 (PD-1 ligand); BTLA (B and T lymphocyte attenuator); CTLA4 (cytotoxic T-lymphocyte associated antigen 4); TIM-3 (T-cell membrane protein 3); LAG3 (lymphocyte activation gene 3); TIGIT (T cell immunoreceptor with Ig and ITIM domains); and Killer Inhibitory Receptors, which can be divided into two classes based on their structural features: i) killer cell immunoglobulin-like receptors (KIRs), and ii)C-type lectin receptors (members of the type II transmembrane receptor family). Other less well-defined immune checkpoints have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

The present invention contemplates the use of the inhibitors of HIF-2α function described herein in combination with inhibitors of the aforementioned immune-checkpoint receptors and ligands, as well as yet-to-be-described immune-checkpoint receptors and ligands. Certain modulators of immune checkpoints are currently approved, and many others are in development. When it was approved for the treatment of melanoma in 2011, the fully humanized CTLA4 monoclonal antibody ipilimumab (YERVOY®; Bristol-Myers Squibb) became the first immune checkpoint inhibitor to receive regulatory approval in the US. Fusion proteins comprising CTLA4 and an antibody (CTLA4-Ig; abatcept (ORENCIA®; Bristol-Myers Squibb)) have been used for the treatment of rheumatoid arthritis, and other fusion proteins have been shown to be effective in renal transplantation patients that are sensitized to Epstein Ban Virus. The next class of immune checkpoint inhibitors to receive regulatory approval were against PD-1 and its ligands PD-L1 and PD-L2. Approved anti-PD-1 antibodies include nivolumab (OPDIVO®; Bristol-Myers Squibb) and pembrolizumab (KEYTRUDA®; Merck) for various cancers, including squamous cell carcinoma, classical Hodgkin lymphoma and urothelial carcinoma. Approved anti-PD-L1 antibodies include avelumab (BAVENCIO, EMD Serono & Pfizer), atezolizumab (TECENTRIQ; Roche/Genentech), and durvalumab (IMFINZI; AstraZeneca) for certain cancers, including urothelial carcinoma. While there are no approved therapeutics targeting TIGIT or its ligands CD155 and CD112, those in development include BMS-986207 (Bristol-Myers Squibb), MTIG7192A/RG6058 (Roche/Genentech), and OMP-31M32 (OncoMed).

In one aspect of the present invention, the claimed HIF-2α inhibitors are combined with an immuno-oncology agent that is (i) an agonist of a stimulatory (including a co-stimulatory) receptor or (ii) an antagonist of an inhibitory (including a co-inhibitory) signal on T cells, both of which result in amplifying antigen-specific T cell responses. Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), B7-H6, and B7-H7 (HHLA2). Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LT13R, LIGHT, DcR3, HVEM, VEGI/TL1A, TRAMP/DR3, EDAR, EDA1, XEDAR, EDA2, TNFR1, Lymphotoxin a/TNF13, TNFR2, TNFa, LT13R, Lymphotoxin a 1132, FAS, FASL, RELT, DR6, TROY, NGFR.

In another aspect, the immuno-oncology agent is a cytokine that inhibits T cell activation (e.g., IL-6, IL-10, TGF-B, VEGF, and other immunosuppressive cytokines) or a cytokine that stimulates T cell activation, for stimulating an immune response.

In one aspect, T cell responses can be stimulated by a combination of the disclosed HIF-2a inhibitors and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and/or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD2. Other agents that can be combined with the HIF-2α inhibitors of the present invention for the treatment of cancer include antagonists of inhibitory receptors on NK cells or agonists of activating receptors on NK cells. For example, compounds herein can be combined with antagonists of KIR, such as lirilumab. As another example, compounds described herein can be combined with lenvatinib or cabozantinib.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/169264; WO14/036357).

In another aspect, the disclosed HIF-2α inhibitors can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY® (ipilimumab) or tremelimumab.

In another aspect, the immuno-oncology agent is a PD-1 antagonist, such as an antagonistic PD-1 antibody. Suitable PD-1 antibodies include, for example, OPDIVO® (nivolumab), KEYTRUDA® (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgGl, called AMP-224. In another embodiment, the agent is zimberelimab.

In another aspect, the immuno-oncology agent is a PD-L1 antagonist, such as an antagonistic PD-L1 antibody. Suitable PD-L1 antibodies include, for example, TECENTRIQ® (atezolizumab; MPDL3280A; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

The present invention encompasses pharmaceutically acceptable salts, acids or derivatives of any of the above.

Examples of therapeutic agents useful in combination therapy for the treatment of cardiovascular and/or metabolic-related diaseses, disorders and conditions include statins (e.g., CRESTOR®, LESCOL®, LIPITOR®, MEVACOR®, PRAVACOL®, and ZOCOR®), which inhibit the enzymatic synthesis of cholesterol; bile acid resins (e.g., COLESTID®, LO-CHOLEST®, PREVALITE®, QUESTRAN®, and WELCHOL®), which sequester cholesterol and prevent its absorption; ezetimibe (ZETIA®), which blocks cholesterol absorption; fibric acid (e.g., TRICOR®), which reduces triglycerides and may modestly increase HDL; niacin (e.g., NIACOR®), which modestly lowers LDL cholesterol and triglycerides; and/or a combination of the aforementioned (e.g., VYTORIN® (ezetimibe with simvastatin). Alternative cholesterol treatments that may be candidates for use in combination with the HIF-2α inhibitors described herein include various supplements and herbs (e.g., garlic, policosanol, and guggul).

The present invention encompasses pharmaceutically acceptable salts, acids or derivatives of any of the above.

Examples of therapeutic agents useful in combination therapy for immune- and inflammatory-related diseases, disorders or conditions include, but are not limited to, the following: non-steroidal anti-inflammatory drug (NSAID) such as aspirin, ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxicam), salicylates (acetyl salicylic acid, sulfasalazine) and the pyrazolones (apazone, bezpiperylon, feprazone, mofebutazone, oxyphenbutazone, phenylbutazone). Other combinations include cyclooxygenase-2 (COX-2) inhibitors.

Other active agents for combination include steroids such as prednisolone, prednisone, methylprednisolone, betamethasone, dexamethasone, or hydrocortisone. Such a combination may be especially advantageous since one or more adverse effects of the steroid can be reduced or even eliminated by tapering the steroid dose required.

Additional examples of active agents that may be used in combinations for treating, for example, rheumatoid arthritis, include cytokine suppressive anti-inflammatory drug(s) (CSAIDs); antibodies to, or antagonists of, other human cytokines or growth factors, for example, TNF, LT, IL-10, IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists such as chimeric, humanized or human TNF antibodies, REMICADE®, HUMERA®, anti-TNF antibody fragments (e.g., CDP870), and soluble p55 or p75 TNF receptors, derivatives thereof, p75TNFRIgG (ENBREL®) or p55TNFR1gG (LENERcEPT®), soluble IL-13 receptor (sIL-13), and also TNFa-converting enzyme (TACE) inhibitors; similarly, IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the HIF-2α inhibitors described herein include interferon-131a (AVONEX®); interferon-131b (BETASERON®); copaxone; hyperbaric oxygen; intravenous immunoglobulin; clabribine; and antibodies to, or antagonists of, other human cytokines or growth factors (e.g., antibodies to CD40 ligand and CD80).

### Dosing

The HIF-2α inhibitors of the present invention may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the HIF-2α inhibitors of the present invention may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the HIF-2α inhibitors contemplated by the present invention may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the desired HIF-2α inhibitor is contained in a “unit dosage form”. The phrase “unit dosage form” refers to physically discrete units, each unit containing a predetermined amount of the HIF-2α inhibitor, either alone or in combination with one or more additional agents, sufficient to produce the desired effect. It will be appreciated that the parameters of a unit dosage form will depend on the particular agent and the effect to be achieved.

### Kits

The present invention also contemplates kits comprising a compound described herein, and pharmaceutical compositions thereof. The kits are generally in the form of a physical structure housing various components, as described below, and may be utilized, for example, in practicing the methods described above.

A kit can include one or more of the compounds disclosed herein (provided in, e.g., a sterile container), which may be in the form of a pharmaceutical composition suitable for administration to a subject. The compounds described herein can be provided in a form that is ready for use (e.g., a tablet or capsule) or in a form requiring, for example, reconstitution or dilution (e.g., a powder) prior to administration. When the compounds described herein are in a form that needs to be reconstituted or diluted by a user, the kit may also include diluents (e.g., sterile water), buffers, pharmaceutically acceptable excipients, and the like, packaged with or separately from the compounds described herein. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### Experimental

The following examples are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: wt=wildtype; bp=base pair(s); kb=kilobase(s); nt=nucleotides(s); aa=amino acid(s); s or sec=second(s); min=minute(s); h or hr=hour(s); ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; IHC=immunohistochemistry; DMEM=Dulbeco's Modification of Eagle's Medium; EDTA=ethylenediaminetetraacetic acid.

### Materials and Methods

The following general materials and methods were used, where indicated, or may be used in the Examples below:

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., N.Y.).

Where the literature contains an assay or experimental procedure, such assay or procedure may serve as an alterantive basis for evaluating the compounds described herein.

All reactions were performed using a Teflon-coated magnetic stir bar at the indicated temperature and were conducted under an inert atmosphere when stated. Reactions were monitored by TLC (silica gel 60 with fluorescence F254, visualized with a short wave/long wave UV lamp) and/or LCMS (Agilent 1100 series LCMS with UV detection at 254 nm using a binary solvent system [0.1% TFA in MeCN/0.1% TFA in H₂O] using either of the following column: Agilent Eclipse Plus C18 [3.5 μm, 4.6 mm i.d.×100 mm]). Flash chromatography was conducted on silica gel using an automated system (CombiFlash RF+ manufactured by Teledyne ISCO), with detection wavelengths of 254 and 280 nm. Reverse phase preparative HPLC was conducted on an Agilent 1260 Infinity series HPLC. Samples were eluted using a binary solvent system (0.1% TFA in MeCN/0.1% TFA in H₂O) with gradient elution on a Gemini C18 110 Å column (21.2 mm i.d.×250 mm) with detection at 254 nm. Final compounds obtained through preparative HPLC were concentrated. Reported yields are isolated yields unless otherwise stated. All assayed compounds were purified to ≥95% purity as determined by LCMS (Agilent 1100 series LCMS with UV detection at 254 nm using a binary solvent system [0.1% TFA in MeCN/0.1% TFA in H₂O] using the following column: Agilent Eclipse Plus C18 column [3.5 μm, 4.6 mm i.d.×100 mm]). ¹H NMR spectra were recorded on a Varian 400 MHz NMR spectrometer equipped with an Oxford AS400 magnet. Chemical shifts (δ) are reported as parts per million (ppm) relative to residual undeuterated solvent as an internal reference.

### EXAMPLES

### Example 1: 3-Fluoro-5-[(7-methanesulfonyl-1H-indazol-4-yl)amino]benzonitrile

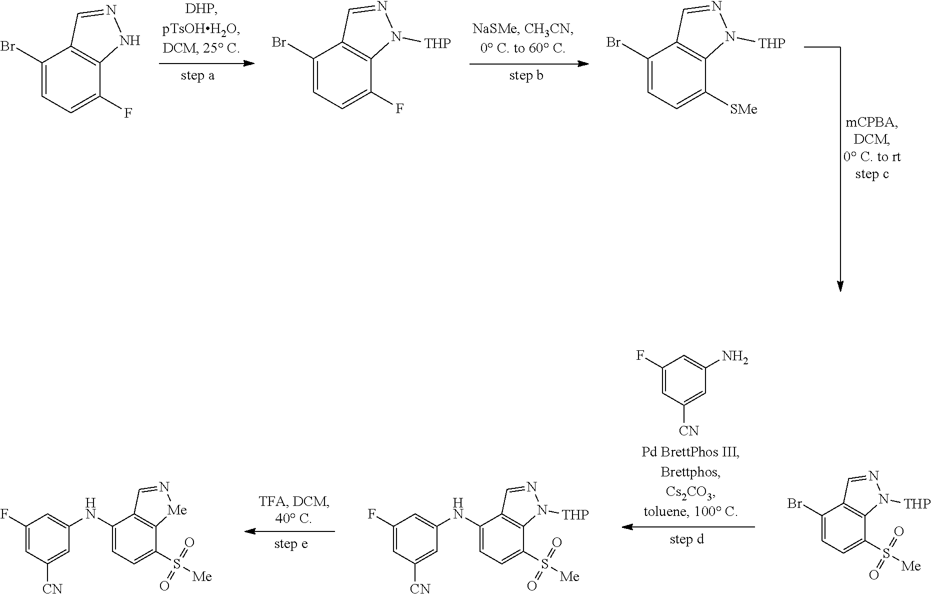

Step a. To a flask containing 4-bromo-7-fluoro-1H-indazole (5.00 g, 23.3 mmol, 1.0 equiv.) was added 3,4-dihydro-2H-pyran (5.92 mL, 69.9 mmol, 3.0 equiv.) and DCM (50 mL). pTsOH H₂O (0.443 g, 2.33 mmol, 10 mol %) was added and the reaction mixture was stirred for 16 h. The reaction was partitioned between sat. aq. NaHCO₃ solution and EtOAc. The aqueous layer was separated and back extracted with additional EtOAc. The organic layers were combined and dried over MgSO₄. Concentration under reduced pressure and purification by flash chromatography (SiO₂, hexanes→20% EtOAc) furnished the THP protected indazole as a yellow oil (4.02 g, 13.3 mmol, 57%).

Step b. To a flask containing the product from step a (2.05 g, 6.88 mmol, 1.0 equiv.) was added CH₃CN (34 mL). The reaction mixture was cooled to 0° C. and NaSMe (0.964 g, 13.8 mmol, 2.0 equiv.) was added. After heating to 60° C. and stirring for 4 h the reaction mixture was quenched with H₂O and diluted with EtOAc. The aqueous layer was separated and back extracted with additional EtOAc. The organic layers were combined, dried over MgSO₄, and concentrated under reduced pressure. The crude thioether was taken onto the next step without further purification.

Step c. A flask containing crude thioether from step b was dissolved in DCM (34 mL) and cooled to 0° C. 75% mCPBA (4.73 g, 20.6 mmol, 3.0 equiv) was added. The reaction mixture was warmed to room temperature and EtOAc (15 mL) was added to render the mixture homogeneous. After 1 h, the reaction mixture was cooled to 0° C. and quenched with sat. aq. Na₂S₂O₃ solution and sat. aq. NaHCO₃ solution and diluted with DCM. The aqueous layer was separated and back extracted with additional DCM. The organic layers were combined and dried over MgSO₄. Concentration under reduced pressure and purification by flash chromatography (SiO₂, hexanes to 50% EtOAc) furnished the indazole sulfone as a white solid (1.55 g, 4.32 mmol, 63% over 2 steps, ESI MS [M+Na]⁺ for C₁₃H₁₅BrN₂O₃S, calcd 381.0, found 381.0.

Step d. To a vial containing the product from step c (500 mg, 1.39 mmol, 1.0 equiv.) was added toluene (7 mL), followed by 3-amino-5-fluoro-benzonitrile (284 mg, 2.10 mmol, 1.5 equiv.), Pd BrettPhos III (63 mg, 0.070 mmol, 5 mol %), BrettPhos (37 mg, 0.070 mmol, 5 mol %), and Cs₂CO₃ (0.903 g, 2.78 mmol, 2.0 equiv.). The reaction mixture was purged with nitrogen, capped, heated to 100° C. and stirred for 15 h. Concentration under reduced pressure and purification by flash chromatography (SiO₂, hexanes 50% EtOAc) furnished the indazole product (548 mg, 1.32 mmol, 95%, ESI MS [M+Na]⁺ for C₂₀H₁₉FN₄O₃S, calcd 437.1, found 437.0).

Step e. The product from step d (300 mg, 0.725 mmol) was dissolved in DCM (4 mL). TFA (2 mL) was added and the reaction mixture was warmed to 40° C. and stirred for 40 min. The reaction was partitioned between sat. aq. NaHCO₃ solution and DCM. The aqueous layer was separated and back extracted with additional DCM. The organic layers were combined and dried over MgSO₄. Concentration under reduced pressure and purification by flash chromatography (SiO₂, DCM to 60% EtOAc) furnished the title compound as a white solid. ¹H NMR (400 MHz, DMSO-d6) δ 13.40 (s, 1H), 9.58 (s, 1H), 8.40 (s, 1H), 7.72 (d, J=8.2 Hz, 1H), 7.59-7.54 (m, 1H), 7.53-7.43 (m, 2H), 7.04 (d, J=8.2 Hz, 1H), 3.27 (s, 3H). ESI MS [M+H]⁺ for C₁₅H₁₁FN₄O₂S, calcd 331.1, found 331.0.

### Example 2: 3-Fluoro-5-[(3-fluoro-7-methanesulfonyl-1H-indazol-4-yl)amino]benzonitrile

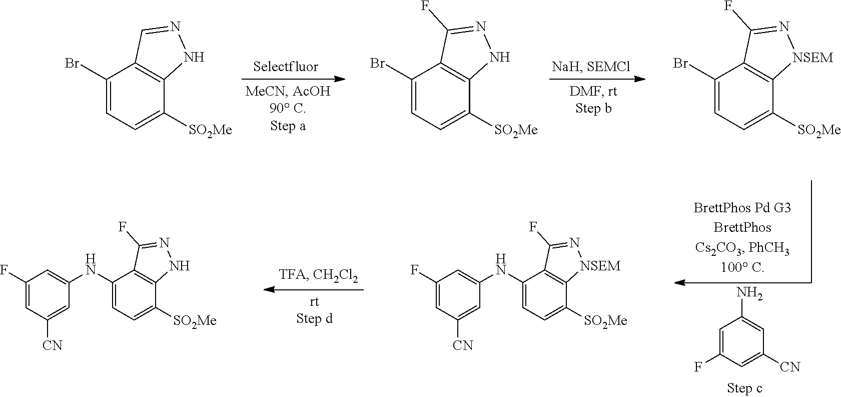 [SMILES: CS(=O)(=O)c1ccc(Br)c2cn[nH]c12 ; CS(=O)(=O)c1ccc(Br)c2c(F)n[nH]c12 ; C[Si](C)(C)CCOCn1nc(F)c2c(Br)ccc(S(C)(=O)=O)c21 ; CS(=O)(=O)c1ccc(Nc2cc(F)cc(C#N)c2)c2c(F)n[nH]c12 ; C[Si](C)(C)CCOCn1nc(F)c2c(Nc3cc(F)cc(C#N)c3)ccc(S(C)(=O)=O)c21 ; N#Cc1cc(N)cc(F)c1]

Step a. To a solution of intermediate 4-bromo-7-methylsulfonyl-1-(oxan-2-yl)indazole (1.0 g, 2.79 mmol, 1.0 equiv.) in acetonitrile (7.8 mL) and acetic acid (0.31 mL) was added Selectfluor (1.97 g, 5.58 mmol, 2.0 equiv.) and the reaction was heated to 90° C. for 5 hours. The reaction mixture was subsequently diluted with H₂O (20 mL) and extracted into EtOAc (3×20 mL). Layers were separated, organic layer was washed with brine and dried over anhydrous Na₂SO₄. Solvent was removed in vacuo to give a crude residue that was purified by column chromatography (SiO₂, gradient 0% to 20% EtOAc in hexanes) to afford 4-bromo-3-fluoro-7-(methanesulfonyl)-1H-indazole (308 mg, 38% yield). ESI MS [M+H]⁺ for C₈H₆BrFN₂O₂S calcd 292.9, found 293.0.

Step b. To a solution of 4-bromo-3-fluoro-7-(methanesulfonyl)-1H-indazole from step a (308 mg, 1.05 mmol, 1.0 equiv.) in DMF (3.2 mL) at 0° C. was added NaH (60% dispersion in oil, 47 mg, 1.16 mmol, 1.1 equiv.) and the reaction mixture was stirred at 0° C. for 30 min. 2-(Trimethylsilyl)ethoxymethyl chloride (0.24 mL, 1.37 mmol, 1.3 equiv.) was then added dropwise and the reaction was warmed to rt overnight. Reaction mixture was then cooled to 0° C., H₂O (5 mL) and EtOAc (20 mL) were added. Layers were separated and the organic layer was washed with H₂O (2×5 ml), brine and dried over anhydrous Na₂SO₄. Solvent was removed in vacuo to give a crude residue that was purified by column chromatography (SiO₂, gradient 0% to 20% EtOAc in hexanes) to afford the desired product (200 mg, 45% yield).

Step c. To a solution product from step b (200 mg, 0.47 mmol, 1.0 equiv.), 3-amino-5-fluorobenzonitrile (78 mg, 0.56 mmol, 1.2 equiv.) and cesium carbonate (309 mg, 0.95 mmol, 2.0 equiv.) in degassed toluene (2.4 mL) under nitrogen was added BrettPhos Pd G3 (40 mg, 0.047 mmol, 0.10 equiv.) and BrettPhos (23 mg, 0.047 mmol, 0.10 equiv.). The reaction vessel was evacuated and refilled with nitrogen. This process was repeated twice and the reaction was heated to 100° C. for 16 hours. At this point, the reaction was filtered over Celite® and the filter cake was washed with EtOAc. Solvent was subsequently removed in vacuo to give a crude residue that was purified by column chromatography (SiO₂, gradient 0% to 30% EtOAc in hexanes) to afford the desired product (100 mg, 44% yield).

Step d. To solution of the product from step c (100 mg, 0.20 mmol) in CH₂Cl₂ (2 mL) was added TFA (2 mL) dropwise. The reaction mixture was stirred at rt for 30 min. Solvent was removed in vacuo to give a crude residue that was purified by reverse phase HPLC (MeCN/H₂O) to provide 3-fluoro-5-[(3-fluoro-7-methanesulfonyl-1H-indazol-4-yl)amino]benzonitrile (10 mg, 14% yield). ¹H NMR (400 MHz, CD₃OD) δ 7.82 (d, J=8.3 Hz, 1H), 7.49 (ddd, J=1.9, 1.3, 0.5 Hz, 1H), 7.43-7.37 (m, 1H), 7.25-7.37 (m, 1H), 6.99 (d, J=8.3 Hz, 1H), 3.18 (s, 3H). ¹⁹F NMR (376 MHz, CD₃OD) δ−131.9,−110.8. ESI MS [M+H]⁺ for C₁₅H₁₀F₂N₄O₂S calcd 349.0, found 349.1.

### Example 3: 3-Fluoro-5-[(7-methanesulfonyl-1H-indazol-4-yl)oxy]benzonitrile

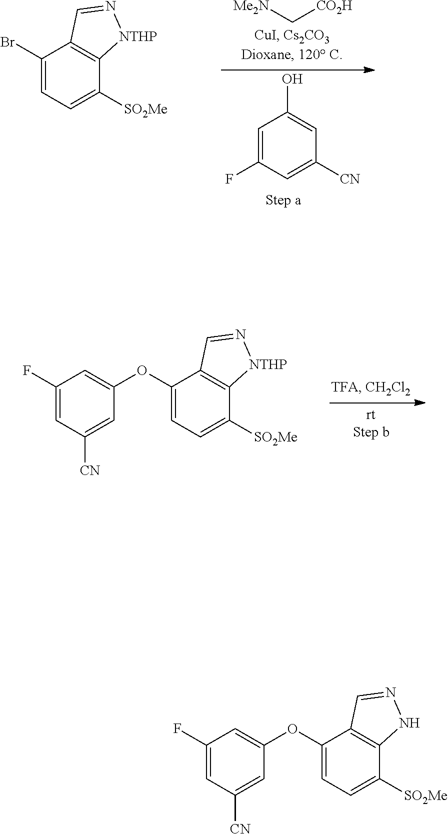

Step a. To a mixture of intermediate 4-bromo-7-methylsulfonyl-1-(oxan-2-yl)indazole (0.99 g, 2.76 mmol, 1.0 equiv.) in degassed dioxane (9.7 mL) was added 3-hydroxy-5-fluorobenzonitrile (454 mg, 3.31 mmol, 1.2 equiv.), N,N-dimethylglycine (85 mg, 0.83 mmol, 0.3 equiv.), Cs₂CO₃ (1.80 g, 5.52 mmol, 2.0 equiv.) and CuI (52 mg, 0.27 mmol, 0.1 equiv.). The reaction was heated to 120° C. for 16 hours. At this point, the reaction was filtered over Celite® and the filter cake was washed with EtOAc. Solvent was subsequently removed in vacuo to give a crude residue that was purified by column chromatography (SiO₂, gradient 0% to 40% EtOAc in hexanes) to afford 3-fluoro-5-[7-methylsulfonyl-1-(oxan-2-yl)indazol-4-yl]oxybenzonitrile (435 mg, 38% yield). ESI MS [M+Na]⁺ for C₂₀H₁₈FN₃O₄S calcd 438.1, found 438.0.

Step b. To solution of the product from step a (40 mg, 0.096 mmol) in CH₂Cl₂ (2 mL) was added TFA (2 mL) dropwise. The reaction mixture was stirred at rt for 30 min. Solvent was removed in vacuo to give a crude residue that was purified by reverse phase HPLC (MeCN/H₂O) to provide 3-fluoro-5-[(7-methanesulfonyl-1H-indazol-4-yl)oxy]benzonitrile (20 mg, 63% yield). ¹H NMR (400 MHz, DMSO-d6) δ 13.72 (s, 1H), 8.26 (s, 1H), 7.89-7.79 (m, 3H), 7.74-7.71 (m, 1H), 7.69-7.63 (m, 1H), 6.75 (d, J=8.1 Hz, 1H), 3.34 (s, 3H). ¹⁹F NMR (376 MHz, DMSO-d6) δ-107.2. ESI MS [M+H]⁺ for C₁₅H₁₀FN₃O₃S calcd 332.0, found 332.1.

### Example 4: 4-(2,4-Difluorophenoxy)-7-methanesulfonyl-1H-indazole

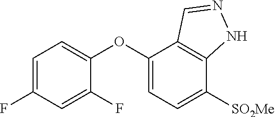 [SMILES: CS(=O)(=O)c1ccc(Oc2ccc(F)cc2F)c2cn[nH]c12]

The title compound was synthesized in a similar fashion to Example 3. ¹H NMR (400 MHz, DMSO-d₆) δ 8.35 (s, 1H), 7.79 (d, J=8.2 Hz, 1H), 7.70-7.52 (m, 2H), 7.34-7.22 (m, 1H), 6.46 (d, J=8.2 Hz, 1H), 3.32 (s, 3H). ¹⁹F NMR (376 MHz, DMSO-d₆) δ-125.5,−112.2. ESI MS [M+H]⁺ for C₁₄H₁₀F₂N₂O₃S calcd 325.0, found 325.1.

### Example 5: 4-(3-Chlorophenoxy)-7-methanesulfonyl-1H-indazole

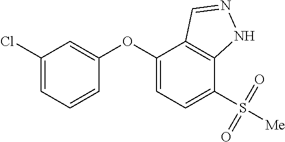 [SMILES: CS(=O)(=O)c1ccc(Oc2cccc(Cl)c2)c2cn[nH]c12]

The title compound was synthesized in a similar fashion to Example 3. ¹H NMR (400 MHz, DMSO-d6) δ 13.68 (s, 1H), 8.22 (s, 1H), 7.83 (d, J=8.2 Hz, 1H), 7.52 (t, J=8.2 Hz, 1H), 7.45-7.38 (m, 2H), 7.26 (ddd, J=8.2, 2.3, 1.0 Hz, 1H), 6.60 (d, J=8.2 Hz, 1H), 3.33 (s, 3H). ESI MS [M+H]⁺ for C₁₄H₁₀Cl₂N₂O₃S, calcd 323.0, found 323.1.

### Example 6: 4-(3,4-Dichlorophenoxy)-7-methanesulfonyl-1H-indazole

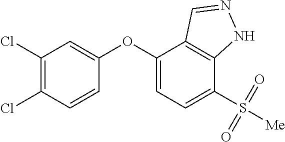 [SMILES: CS(=O)(=O)c1ccc(Oc2ccc(Cl)c(Cl)c2)c2cn[nH]c12]

The title compound was synthesized in a similar fashion to Example 3. ¹H NMR (400 MHz, DMSO-d6) δ 13.69 (s, 1H), 8.26 (s, 1H), 7.82 (d, J=8.2 Hz, 1H), 7.76 (d, J=8.6 Hz, 1H), 7.68 (d, J=2.7 Hz, 1H), 7.30 (dd, J=8.8, 2.8 Hz, 1H), 6.66 (d, J=8.2 Hz, 1H), 3.32 (s, 3H). ESI MS [M+H]⁺ for C₁₄H₁₀Cl₂N₂O₃S, calcd 357.0, found 357.0.

### Example 7: 4-(3-Chloro-5-fluorophenoxy)-7-methanesulfonyl-1H-indazole

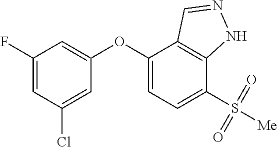 [SMILES: CS(=O)(=O)c1ccc(Oc2cc(F)cc(Cl)c2)c2cn[nH]c12]

The title compound was synthesized in a similar fashion to Example 3. ¹H NMR (400 MHz, DMSO-d6) δ 13.71 (s, 1H), 8.25 (s, 1H), 7.84 (d, J=8.1 Hz, 1H), 7.44-7.39 (m, 1H), 7.31-7.22 (m, 2H), 6.73 (d, J=8.2 Hz, 1H), 3.32 (s, 3H). ESI MS [M+H]⁺ for C₁₄H₁₀ClFN₂O₃S, calcd 341.0, found 341.0.

### Example 8: 3-[(3-Chloro-7-methanesulfonyl-1H-indazol-4-yl)oxy]-5-fluorobenzonitrile

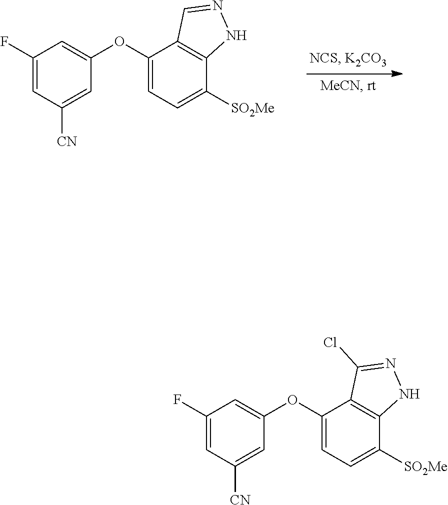 [SMILES: CS(=O)(=O)c1ccc(Oc2cc(F)cc(C#N)c2)c2cn[nH]c12 ; CS(=O)(=O)c1ccc(Oc2cc(F)cc(C#N)c2)c2c(Cl)n[nH]c12]

To a solution of 3-fluoro-5-[(7-methanesulfonyl-1H-indazol-4-yl)oxy]benzonitrile (Example 3) (14.6 mg, 0.032 mmol, 1.0 equiv.) in MeCN (1 mL) was added K₂CO₃ (4.6 mg, 0.032 mmol, 1.0 equiv.) and N-chlorosuccinimide (9.0 mg, 0.065 mmol, 2.0 equiv.). The reaction was stirred at rt for 16 hours. At this point, solvent was removed in vacuo to give a crude residue that was purified by reverse phase HPLC (MeCN/H₂O) to provide 3-[(3-chloro-7-methanesulfonyl-1H-indazol-4-yl)oxy]-5-fluorobenzonitrile (5.0 mg, 41% yield). ¹H NMR (400 MHz, CD₃OD) δ 7.95 (d, J=8.2 Hz, 1H), 7.51-7.45 (m, 1H), 7.45-7.42 (m, 1H), 7.39-7.34 (m, 1H), 6.79 (d, J=8.2 Hz, 1H), 3.23 (3H, s). ¹⁹F NMR (376 MHz, CD₃OD) δ-108.8. ESI MS [M+H]⁺ for C₁₅H₉ClFN₃O₃S calcd 366.0, found 366.1.

### Example 9: 3-Chloro-4-(3-chloro-5-fluorophenoxy)-7-methanesulfonyl-1H-indazole

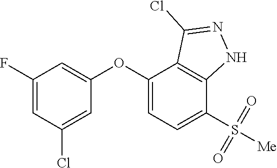 [SMILES: CS(=O)(=O)c1ccc(Oc2cc(F)cc(Cl)c2)c2c(Cl)n[nH]c12]

The title compound was synthesized in a similar fashion to Example 8. ¹H NMR (400 MHz, DMSO-d6) δ 13.87 (s, 1H), 7.89 (d, J=8.2 Hz, 1H), 7.43-7.36 (m, 1H), 7.28-7.20 (m, 2H), 6.77 (d, J=8.2 Hz, 1H), 3.34 (s, 3H). ESI MS [M+H]⁺ for C₁₄H₉Cl₂FN₂O₃S, calcd 375.0, found 375.0.

### Example 10: 4-(3-Cyano-5-fluorophenoxy)-7-methanesulfonyl-1H-indazole-3-carbonitrile

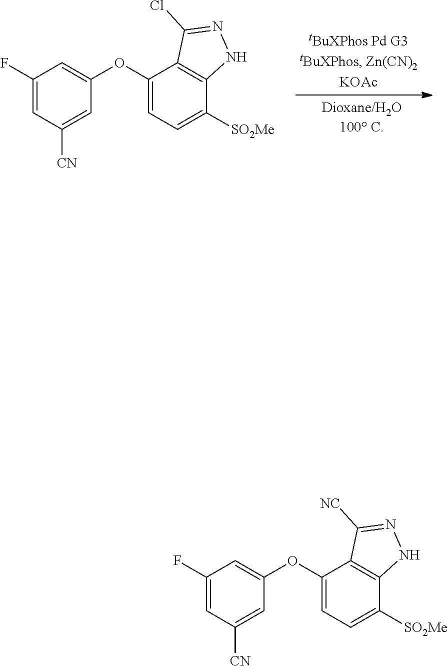 [SMILES: CS(=O)(=O)c1ccc(Oc2cc(F)cc(C#N)c2)c2c(Cl)n[nH]c12 ; CS(=O)(=O)c1ccc(Oc2cc(F)cc(C#N)c2)c2c(C#N)n[nH]c12]

To a solution of 3-[(3-chloro-7-methanesulfonyl-1H-indazol-4-yl)oxy]-5-fluorobenzonitrile (example 8, 19 mg, 0.052 mmol, 1.0 equiv.) in degassed dioxane (0.10 mL) was added Zn(CN)₂ (4.1 mg, 0.035 mmol, 0.66 equiv.) followed by ^{t}BuXPhos Pd G3 (4.2 mg, 0.0052 mmol, 0.1 equiv.) and ^{t}BuXPhos (2.3 mg, 0.0052 mmol, 0.1 equiv.) and degassed KOAc solution in H₂O (0.0625N, 0.1 mL, 0.12 equiv.). The reaction was heated to 100° C. for 2 hours. At this point, solvent was removed in vacuo to give a crude residue that was purified by reverse phase HPLC (MeCN/H₂O) to provide 4-(3-cyano-5-fluorophenoxy)-7-methanesulfonyl-1H-indazole-3-carbonitrile (10 mg, 53% yield). ¹H NMR (400 MHz, DMSO-d₆) δ 7.96 (d, J=8.2 Hz, 1H), 7.88-7.81 (m, 1H), 7.80-7.69 (m, 2H), 6.93 (d, J=8.2 Hz, 1H), 3.36 (s, 3H). ¹⁹F NMR (376 MHz, DMSO-d6) δ-107.1. ESI MS [M+H]⁺ for C₁₆H₉FN₄O₃S calcd 357.0, found 357.1.

### Example 11: 3-Fluoro-5-{[3-(hydroxymethyl)-7-methanesulfonyl-1H-indazol-4-yl]oxy}benzonitrile

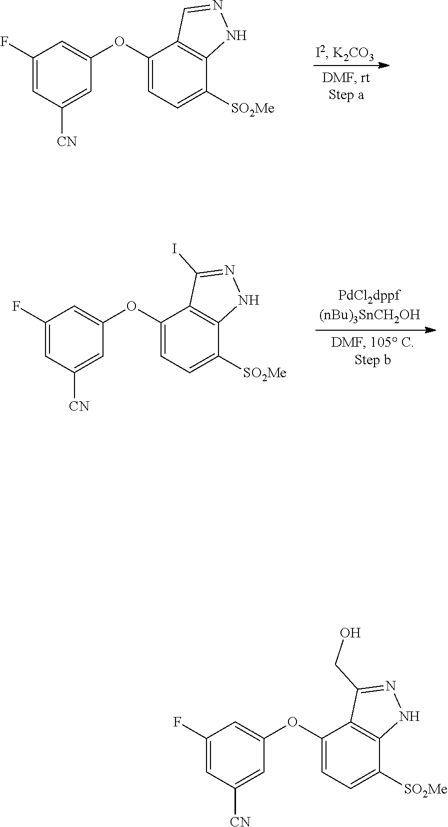 [SMILES: CS(=O)(=O)c1ccc(Oc2cc(F)cc(C#N)c2)c2cn[nH]c12 ; CS(=O)(=O)c1ccc(Oc2cc(F)cc(C#N)c2)c2c(I)n[nH]c12 ; CS(=O)(=O)c1ccc(Oc2cc(F)cc(C#N)c2)c2c(CO)n[nH]c12]

Step a. To a solution of 3-fluoro-5-[(7-methanesulfonyl-1H-indazol-4-yl)oxy]benzonitrile (example 8) (435 mg, 1.04 mmol, 1.0 equiv.) in DMF (4 mL) was added K₂CO₃ (287 mg, 2.08 mmol, 2.0 equiv.) and I₂ (529 mg, 2.08 mmol, 2.0 equiv.). The reaction was heated to 50° C. for 3 hours. At this point, reaction was diluted with EtOAc (30 mL), washed with saturated aqueous Na₂S₂O₃ and brine. Layers were separated, organic layer was dried over anhydrous Na₂SO₄. Solvent was removed in vacuo to give a crude residue that was purified by column chromatography (SiO₂, gradient 0% to 30% EtOAc in hexanes) to afford 3-[(3-iodo-7-trifluoromethanesulfonyl-1H-indazol-4-yl)oxy]-5-fluorobenzonitrile (330 mg, 70% yield). ESI MS [M+H]⁺ for C₁₅H₉IFN₃O₃S calcd 457.9, found 458.0.

Step b. To a solution of 3-[(3-iodo-7-trifluoromethanesulfonyl-1H-indazol-4-yl)oxy]-5-fluorobenzonitrile from a (40 mg, 0.087 mmol, 1.0 equiv.) in degassed DMF (0.44 mL) was added (tributylstannyl)methanol (42 mg, 0.13 mmol, 1.5 equiv.) and PdCl₂dppf (9.5 mg, 0.013 mmol, 0.15 equiv.). Reaction was heated to 105° C. for 4 hours. At this point, solvent was removed in vacuo to give a crude residue that was purified by reverse phase HPLC (MeCN/H₂O) to provide 3-fluoro-5-{[3-(hydroxymethyl)-7-methanesulfonyl-1H-indazol-4-yl]oxy}benzonitrile. ¹H NMR (400 MHz, DMSO-d6) δ 7.85-7.82 (m, 1H), 7.77 (d, J=9.0 Hz, 1H), 7.75-7.73 (m, 1H), 7.72-7.68 (m, 1H), 6.82 (s, 2H), 6.52 (s, 1H), 6.27 (d, J=8.9 Hz, 1H), 3.18 (s, 3H). ¹⁹F NMR (376 MHz, DMSO-d6) δ-107.1. ESI MS [M+H]⁺ for C₁₆H₁₂FN₃O₄S calcd 361.0, found 361.0.

### Example 12: 5-[7-(Difluoromethylsulfonyl)-1H-indazol-4-yloxy]-3-fluorobenzonitrile

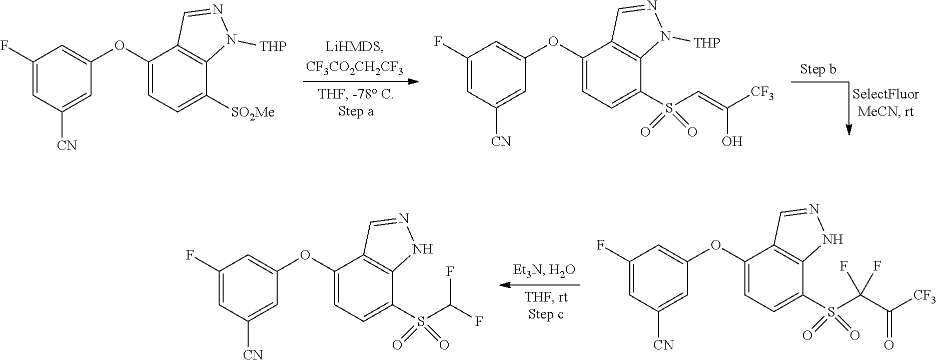

Step a. A solution of 3-fluoro-5-[7-methylsulfonyl-1-(oxan-2-yl)indazol-4-yl]oxybenzonitrile (product of example 3, step a) (300 mg, 0.72 mmol) in THF (1 mL) was added dropwise to a solution of LiHMDS (1M/THF, 0.87 mL) in degassed THF (3.6 mL) at −78° C. After 45 minutes, 2,2,2-Trifluoroethyl trifluoroacetate (0.21 g, 1.08 mmol) was added dropwise. The resulting mixture was stirred at −78° C. for 15 minutes then quenched with 1M sulfuric acid and stirred for 1 hour at room temperature. The crude product was used without further purification.

Step b. The product from Step a (0.72 mmol) in MeCN (1.9 mL) at room temperature was treated with Selectfluor (561 mg, 1.6 mmol). The mixture was stirred for 48 hours then diluted with EtOAc and filtered through Celite®. Column chromatography (SiO₂, 0→30% EtOAc/Hex) afforded the desired product (34 mg, 10% yield, two-steps). ESI MS [M+H]⁺ for C₁₇H₇F₆N₃O₄S, calcd 464.0, found 464.0.

Step c. The product from step b (34 mg, 0.07 mmol) was dissolved in THF (1 mL). One drop of water was added followed by Et₃N (0.03 mL, 0.21 mmol). After complete hydrolysis was observed, the reaction was concentrated onto celite and purified by column chromatography (SiO₂, 0→40% EtOAc/Hex) to afford the desired product (15 mg, 58% yield) as a white solid. ¹H NMR (400 MHz, Chloroform-d) δ 11.13 (s, 1H), 8.17 (s, 1H), 7.89 (d, J=8.3 Hz, 1H), 7.38-7.29 (m, 2H), 7.21 (dt, J=8.8, 2.3 Hz, 1H), 6.68 (d, J=8.3 Hz, 1H), 6.30 (t, J=53.5 Hz, 1H). ESI MS [M+H]⁺ for C₁₅H₈F₃N₃O₃S calcd 368.0, found 368.0.

### Example 13: 5-[7-(Difluoromethylsulfonyl)-3-iodo-1H-indazol-4-yloxy]-3-fluorobenzonitrile

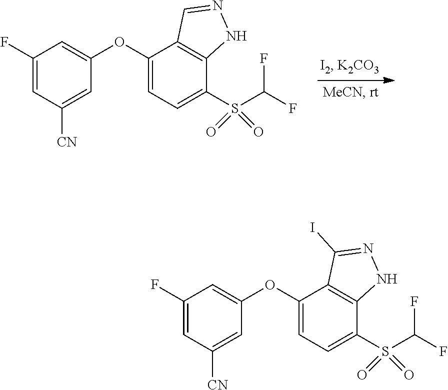 [SMILES: N#Cc1cc(F)cc(Oc2ccc(S(=O)(=O)C(F)F)c3[nH]ncc23)c1 ; N#Cc1cc(F)cc(Oc2ccc(S(=O)(=O)C(F)F)c3[nH]nc(I)c23)c1]

To a solution of 5-[7-(difluoromethylsulfonyl)-1H-indazol-4-yloxy]-3-fluorobenzonitrile (example 12) (61 mg, 0.17 mmol) in MeCN (1.6 mL) was treated with K₂CO₃ (46 mg, 0.34 mmol) followed by 12 (85 mg, 0.34 mmol) at room temperature. After 3 hours the reaction was filtered and concentrated under reduced pressure. Purification by column chromatography (SiO₂, 0→50% EtOAc/Hex) afforded the desired product as a white solid. ¹H NMR (400 MHz, Chloroform-d) δ 11.18 (s, 1H), 7.89 (d, J=8.4 Hz, 1H), 7.37-7.29 (m, 2H), 7.21 (dt, J=8.8, 2.3 Hz, 1H), 6.64 (d, J=8.3 Hz, 1H), 6.29 (t, J=53.5 Hz, 1H). ESI MS [M+H]⁺ for C₁₅H₇F₃IN₃O₃S calcd 393.9, found 494.0.

### Example 14: 3-Fluoro-5-[7-(trifluoromethyl)-1H-indazol-4-ylamino]benzonitrile

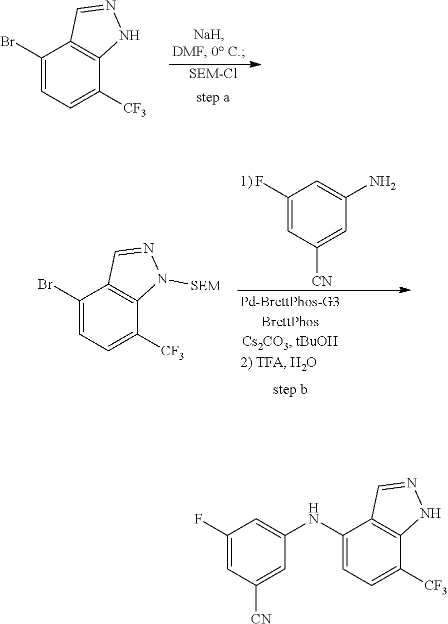 [SMILES: FC(F)(F)c1ccc(Br)c2cn[nH]c12 ; "SEM-Cl" ; C[Si](C)(C)CCOCn1ncc2c(Br)ccc(C(F)(F)F)c21 ; N#Cc1cc(N)cc(F)c1 ; N#Cc1cc(F)cc(Nc2ccc(C(F)(F)F)c3[nH]ncc23)c1]

Step a: To a solution of 4-bromo-7-(trifluoromethyl)-1H-indazole (102 mg, 0.38 mmol) in DMF (3.8 mL) at 0° C. was added sodium hydride (60 wt % dispersion in oil, 18 mg, 0.46 mmol). The reaction was stirred for 15 minutes at 0° C. then 2-(Trimethylsilyl)ethoxymethyl chloride (0.081 mL, 0.46 mmol) was added and was reaction was stirred for 30 minutes then quenched with H₂O. The reaction was diluted with EtOAc and H₂O. The organics were washed with water (2×) and brine, dried over MgSO₄ and concentrated under reduced pressure. The crude product obtained was used without further purification.

Step b: The product from step a (0.38 mmol), 3-amino-5-fluorobenzonitrile (78 mg, 0.57 mmol), Pd-BrettPhos-G3 (36 mg, 0.04 mmol), BrettPhos (21 mg, 0.04 mmol) and Cs₂CO₃ (248 mg, 0.76 mmol) were combined in a flask and evacuated and backfilled with N₂ several times. Tert-butanol (3.8 mL) was added and mixture sealed and heated to 85° C. overnight. After cooling to room temperature, the reaction was diluted with EtOAc and H₂O. The organics were washed with water (2×) and brine, dried over MgSO₄ and concentrated under reduced pressure. The crude product was reconstituted in 90% v/v TFA/H₂O and stirred at room temperature for 30 minutes. The reaction was diluted with toluene and concentrated under reduced pressure. Purification by preparative HPLC (C18, MeCN/H₂O, 0.1% TFA gradient) provide the desired product (21 mg, 17%, two-steps). ¹H NMR (400 MHz, DMSO-d6) δ 13.54 (s, 1H), 9.41 (s, 1H), 8.33 (s, 1H), 7.61-7.54 (m, 1H), 7.49 (s, 1H), 7.45-7.34 (m, 2H), 6.99 (d, J=8.0 Hz, 1H). ESI MS [M+H]⁺ for C₁₅H₈F₄N₄, calcd 321.1, found 321.1.

### Example 15: 3-Fluoro-5-[(7-trifluoromethanesulfonyl-1H-indazol-4-yl)oxy]benzonitrile

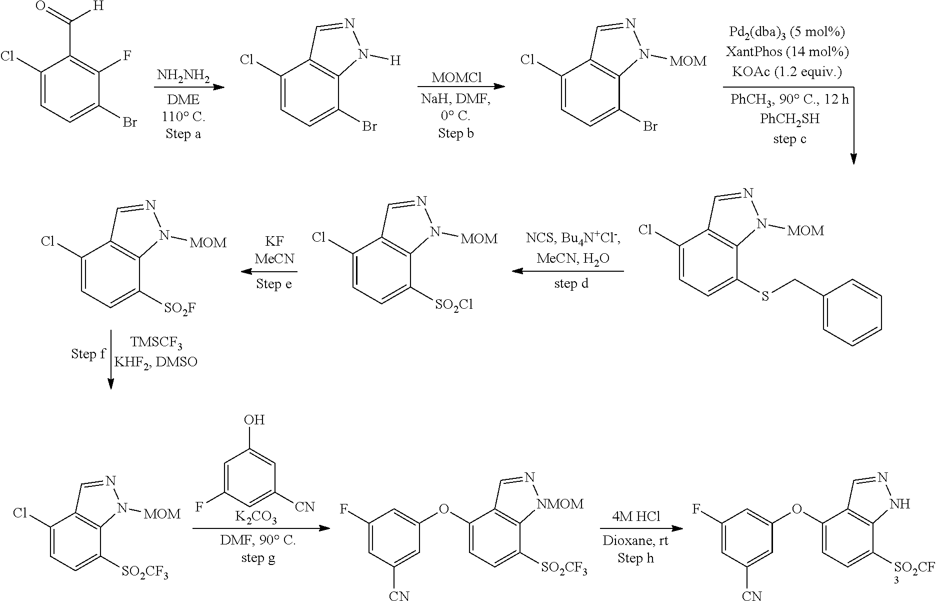

Step a. A mixture of 3-bromo-6-chloro-2-fluorobenzaldehyde (25 g, 105 mmol) and Hydrazine monohydrate (50 mL) in 1,2-dimethoxyethane (125 mL) was heated at reflux for overnight. Reaction mixture was cooled to room temperature, concentrated. The residue was diluted with EtOAc and washed with H₂O. Organic layer was separated, dried over MgSO₄, filtered and evaporated to give yellowish-white solid. Crude product was washed with hexanes and used directed in the next step (22.9 g, 94%).

Step b. The product from step a (22.9 g, 99.1 mmol) was dissolved in DMF (220 mL) and cooled to 0° C. (ice bath), NaH (60% in mineral oil) (5.15 g, 128.8 mmol, 1.3 equiv.) was added portion wise slowly. The reaction mixture was stirred at 0° C. for 30 min, then a solution of chloromethyl methyl ether (10.4 g, 128.8 mmol, 1.3 equiv.) in DMF (30 mL) was added dropwise at 0° C. Let it warm up to room temperature and stir for 3 h. The reaction mixture was carefully quenched with H₂O (1.5 L). The solid was collected and washed with H₂O. Used in next step without further purification.

Step c. The product from step b (99.1 mmol) was combined with Xantphos (5.73 g, 9.9 mmol, 0.1 equiv.), Pd₂(dba)₃ (4.54 g, 4.96 mmol, 0.05 equiv.), DIPEA (34.5 mL, 198.2 mmol, 2.0 equiv.) and benzyl mercaptan (12.2 mL, 104 mmol, 1.05 equiv.) under N₂ in degassed toluene (250 mL). The reaction mixture was stirred at 100° C. for 8 h. Upon cooling, the solids were removed by filtration through Celite®. The Celite® was washed with EtOAc. The solution was concentrated. The crude material was purified by column chromatography (SiO₂, 0 to 25% EtOAc in hexanes) to afford the desired product (25.8 g; 82% for two steps).

Step d. The product from Step c (25.8 g, 80.8 mmol,) was combined with tetrabutylammonium chloride (56.1 g, 202 mmol, 2.5 equiv.) and H₂O (3.64 g, 202 mmol, 2.5 equiv.) in MeCN (270 mL). N-chlorosuccinimide (28.1 g, 210 mmol, 2.6 equiv.) was added portion wise. The reaction mixture was stirred at room temperature for 30 min, then more N-chlorosuccinimide (5.4 g, 40.4 mmol, 0.5 equiv.) was added to the reaction mixture. Let it stir for 15 min, followed by another N-chlorosuccinimide (5.4 g, 40.4 mmol, 0.5 equiv.). Let it stir for 15 min. Reaction mixture was concentrated. The residue was diluted with EtOAc and washed with H₂O. Organic layer was separated, dried over MgSO₄, filtered and evaporated. The crude material was purified by column chromatography (SiO₂, 0 to 25% EtOAc in hexanes) to afford the desired product (14.5 g; 61%).

Step e. The product from Step d (14.5 g, 49 mmol,) was combined with 18-crown-6 (0.65 g, 2.4 mmol, 0.05 equiv.) and KF (11.4 g, 197 mmol, 4.0 equiv.) in MeCN (75 mL). After stirred at room temperature for 2 h, the reaction mixture was diluted with H₂O, extracted with EtOAc. Organic layer was separated, washed with H₂O, dried over MgSO₄, filtered and evaporated. The crude material was purified by column chromatography (SiO₂, 0 to 25% EtOAc in hexanes) to afford the desired product (8.0 g; 56%).

Step f. The product from Step e (4.2 g, 14.4 mmol,) was combined with KHF₂ (0.34 g, 4.32 mmol, 0.3 equiv.) in DMSO (30 mL) under N₂. The mixture was sonicated for 2 min. TMSCF₃ (4.08 g, 28.8 mmol, 2.0 equiv.) was added dropwise to the mixture. After stirred at room temperature for 20 min, the reaction mixture was diluted with H₂O, extracted with EtOAc. Organic layer was separated, washed with H₂O×4, dried over MgSO₄, filtered and evaporated. The crude material was purified by column chromatography (SiO₂, 0 to 25% EtOAc in hexanes) to afford the desired product (4.2 g; 88%). ¹H NMR (400 MHz, Chloroform-d) δ 8.35 (s, 1H), 8.19 (d, J=8.2 Hz, 1H), 7.47-7.37 (d, J=8.2 Hz, 1H), 6.07 (s, 2H), 3.36 (s, 3H).

Step g. To a mixture of the product from Step f (400 mg, 1.21 mmol, 1.0 equiv.) in DMF (7.1 mL) was added 3-hydroxy-5-fluorobenzonitrile (315 mg, 2.42 mmol, 2.0 equiv.) and potassium carbonate (334 mg, 2.42 mmol, 2.0 equiv.). The reaction was heated to 90° C. for 5 h. Upon completion, the reaction was diluted with H₂O and extracted into EtOAc (2×30 mL). Layers were separated, organic layer was washed with brine and dried over anhydrous Na₂SO₄. Solvent was removed in vacuo to give a crude residue that was purified by column chromatography (SiO₂, gradient 0% to 40% EtOAc in hexanes) to afford 3-fluoro-5-{[1-(methoxymethyl)-7-(trifluoromethanesulfonyl)-1H-indazol-4-yl]oxy}benzonitrile (350 mg, 74% yield). ESI MS [M+H]⁺ for C₁₅H₁₁F₄N₃O₄S calcd 430.0, found 430.1.

Step h. To a solution of intermediate from Step g (350 mg, 0.80 mmol) was added 4N HCl in dioxane (5 mL) and reaction was stirred at rt for 1 hour. Solvent was subsequently removed in vacuo and the crude residue was purified by column chromatography (SiO₂, gradient 0% to 50% EtOAc in hexanes) to provide 3-fluoro-5-[(7-trifluoromethanesulfonyl-1H-indazol-4-yl)oxy]benzonitrile (265 mg, 86% yield). ¹H NMR (400 MHz, CDCl₃) δ 11.21 (s, 1H), 8.23 (s, 1H), 7.96 (d, J=8.4 Hz, 1H), 7.45-7.31 (m, 2H), 7.26-7.22 (m, 1H), 6.68 (d, J=8.4 Hz, 1H). ¹⁹F NMR (376 MHz, CDCl₃) δ-104.8,−79.0. ESI MS [M+H]⁺ for C₁₅H₇F₄N₃O₃S calcd 386.0, found 386.0.

### Example 16: 4-Phenoxy-7-(trifluoromethylsulfonyl)-1H-indazole

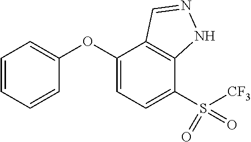 [SMILES: O=S(=O)(c1ccc(Oc2ccccc2)c2cn[nH]c12)C(F)(F)F]

The title compound was synthesized in a similar fashion to Example 15. ¹H NMR (400 MHz, Chloroform-d) δ 8.12 (s, 1H), 7.85 (d, J=8.5 Hz, 1H), 7.54-7.44 (m, 2H), 7.40-7.30 (m, 1H), 7.23-7.16 (m, 2H), 6.58 (d, J=8.5 Hz, 1H). ESI MS [M+H]⁺ for C₁₄H₉F₃N₂O₃S; calcd 343.0, found 343.0.

### Example 17: 4-(p-Chlorophenoxy)-7-(trifluoromethylsulfonyl)-1H-indazole

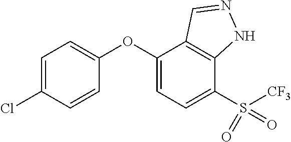 [SMILES: O=S(=O)(c1ccc(Oc2ccc(Cl)cc2)c2cn[nH]c12)C(F)(F)F]

The title compound was synthesized in a similar fashion to Example 15. ¹H NMR (400 MHz, Chloroform-d) δ 8.17 (s, 1H), 7.86 (d, J=8.5 Hz, 1H), 7.51-7.40 (m, 2H), 7.19-7.10 (m, 2H), 6.57 (d, J=8.5 Hz, 1H). ESI MS [M+H]⁺ for C₁₄H₈ClF₃N₂O₃S; calcd 376.9, found 377.0.

### Example 18: 4-(p-Fluorophenoxy)-7-(trifluoromethylsulfonyl)-1H-indazole

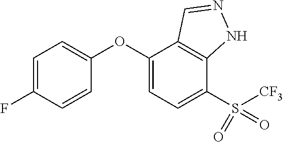 [SMILES: O=S(=O)(c1ccc(Oc2ccc(F)cc2)c2cn[nH]c12)C(F)(F)F]

The title compound was synthesized in a similar fashion to Example 15. ¹H NMR (400 MHz, Chloroform-d) δ 8.16 (s, 1H), 7.85 (d, J=8.5 Hz, 1H), 7.18 (d, J=6.2 Hz, 4H), 6.53 (d, J=8.5 Hz, 1H). ESI MS [M+H]⁺ for C₁₄H₈F₄N₂O₃S; calcd 361.0, found 361.0.

### Example 19: p[7-(Trifluoromethylsulfonyl)-1H-indazol-4-yloxy]benzonitrile

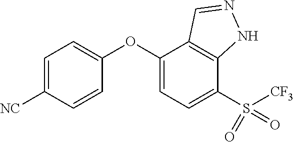 [SMILES: N#Cc1ccc(Oc2ccc(S(=O)(=O)C(F)(F)F)c3[nH]ncc23)cc1]

The title compound was synthesized in a similar fashion to Example 15. ¹H NMR (400 MHz, Chloroform-d) δ 8.18 (s, 1H), 7.92 (d, J=8.4 Hz, 1H), 7.85-7.75 (m, 2H), 7.36-7.28 (m, 2H), 6.67 (d, J=8.4 Hz, 1H). ESI MS [M+H]⁺ for C₁₅H₈F₃N₃O₃S; calcd 368.0, found 368.0.

### Example 20: 4-(p-Methoxyphenoxy)-7-(trifluoromethylsulfonyl)-1H-indazole

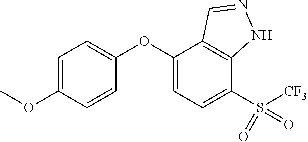 [SMILES: COc1ccc(Oc2ccc(S(=O)(=O)C(F)(F)F)c3[nH]ncc23)cc1]

The title compound was synthesized in a similar fashion to Example 15. ¹H NMR (400 MHz, Chloroform-d) δ 8.11 (s, 1H), 7.83 (d, J=8.5 Hz, 1H), 7.17-7.08 (m, 2H), 7.03-6.94 (m, 2H), 6.55 (d, J=8.5 Hz, 1H), 3.85 (s, 3H). ESI MS [M+H]⁺ for C₁₅H₁₁F₃N₂O₄S; calcd 373.0, found 373.1.

### Example 21: 4-[(6-Methylpyrazin-2-yl)oxy]-7-trifluoromethanesulfonyl-1H-indazole

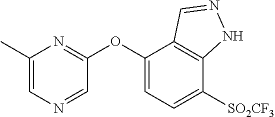

The title compound was synthesized in a similar fashion to Example 15. ¹H NMR (400 MHz, CD₃OD) δ 8.53-8.46 (m, 1H), 8.45-8.40 (m, 1H), 8.20 (s, 1H), 8.11 (d, J=8.4 Hz, 1H), 7.20 (d, J=8.4 Hz, 1H), 2.42 (s, 3H). ¹⁹F NMR (376 MHz, CD₃OD) δ−81.1. ESI MS [M+H]⁺ for C₁₃H₉F₃N₄O₃S calcd 359.0, found 359.1.

### Example 22: 5-[(7-Trifluoromethanesulfonyl-1H-indazol-4-yl)oxy]benzene-1,3-dicarbonitrile

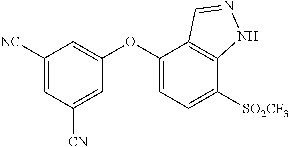

The title compound was synthesized in a similar fashion to Example 15. ¹H NMR (400 MHz, CD₃OD) δ 8.34 (s, 1H), 8.20-8.19 (m, 1H), 8.11-8.09 (m, 2H), 8.07 (d, J=8.4 Hz, 1H), 6.78 (d, J=8.4 Hz, 1H). ¹⁹F NMR (376 MHz, CD₃OD) δ−80.9. ESI MS [M+H]⁺ for C₁₆H₇F₃N₄O₃S calcd 393.0, found 393.0.

### Example 23: 4-[(5-Chloropyridin-3-yl)oxy]-7-trifluoromethanesulfonyl-1H-indazole

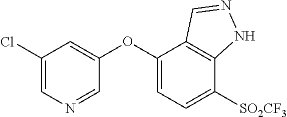

The title compound was synthesized in a similar fashion to Example 15. ¹H NMR (400 MHz, CD₃OD) δ 8.60 (dd, J=2.1, 0.5 Hz, 1H), 8.56 (dd, J=2.4, 0.5 Hz, 1H), 8.34 (s, 1H), 8.06 (d, J=8.4 Hz, 1H), 7.99 (dd, J=2.0 Hz, 1H), 6.75 (d, J=8.4 Hz, 1H). ¹⁹F NMR (376 MHz, CD₃OD) δ−81.1. ESI MS [M+H]⁺ for C₁₃H₇ClF₃N₃O₃S calcd 377.9, found 378.0.

### Example 24: 5-[(7-Trifluoromethanesulfonyl-1H-indazol-4-yl)oxy]pyridine-3-carbonitrile

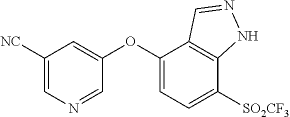

The title compound was synthesized in a similar fashion to Example 15. ¹H NMR (400 MHz, CD₃OD) δ 8.92 (dd, J=1.7, 0.5 Hz, 1H), 8.88 (dd, J=2.7, 0.5 Hz, 1H), 8.37 (s, 1H), 8.28 (dd, J=2.7, 1.7 Hz, 1H), 8.07 (d, J=8.4 Hz, 1H), 6.78 (d, J=8.4 Hz, 1H). ¹⁹F NMR (376 MHz, CD₃OD) δ−77.7. ESI MS [M+H]⁺ for C₁₄H₇F₃N₄O₃S calcd 369.0, found 369.0.

### Example 25: 4-(3,5-Difluorophenoxy)-7-trifluoromethanesulfonyl-1H-indazole

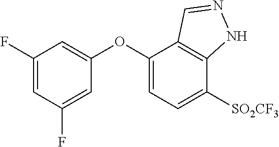

The title compound was synthesized in a similar fashion to Example 15. ¹H NMR (400 MHz, CD₃OD) δ 8.28 (s, 1H), 8.05 (d, J=8.5 Hz, 1H), 7.07-6.96 (m, 3H), 6.78 (d, J=8.5 Hz, 1H). ¹⁹F NMR (376 MHz, CD₃OD) δ-108.9,−81.2. ESI MS [M+H]⁺ for C₁₄H₇F₅N₂O₃S calcd 379.0, found 379.0.

### Example 26: 4-(3,5-Difluorophenoxy)-7-(trifluoromethyl)-1H-indazole

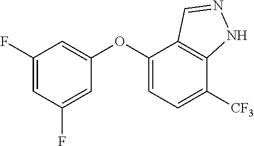 [SMILES: Fc1cc(F)cc(Oc2ccc(C(F)(F)F)c3[nH]ncc23)c1]

The title compound was synthesized in a similar fashion to Example 15. ¹H NMR (400 MHz, DMSO-d6) δ 13.87 (s, 1H), 8.20 (s, 1H), 7.73 (d, J=8.0 Hz, 1H), 7.35-7.17 (m, 1H), 7.13-7.04 (m, 2H), 6.71 (d, J=8.1 Hz, 1H). ¹⁹F NMR (376 MHz, DMSO-d6) δ-107.8,−107.7,−59.6. ESI MS [M+H]⁺ for C₁₄H₇F₅N₂O calcd 315.0, found 315.1.

### Example 27: 3-Chloro-5-[[7-(trifluoromethylsulfonyl)-1H-indazol-4-yl]oxy]benzonitrile

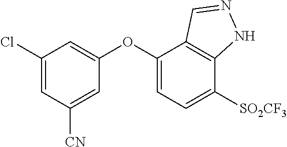

The title compound was synthesized in a similar fashion to Example 15. ¹H NMR (400 MHz, Chloroform-d) δ 8.24 (s, 1H), 7.96 (dt, J=8.4, 0.5 Hz, 1H), 7.65-7.59 (m, 1H), 7.48 (dd, J=2.3, 1.9 Hz, 1H), 7.42 (dd, J=2.3, 1.3 Hz, 1H), 6.64 (d, J=8.4 Hz, 1H). ESI MS [M+H]⁺ for C₁₅H₇ClF₃N₃O3_{S}, calcd 402.7, found 402.0.

### Example 28: 3-Methyl-5-[[7-(trifluoromethylsulfonyl)-1H-indazol-4-yl]oxy]benzonitrile

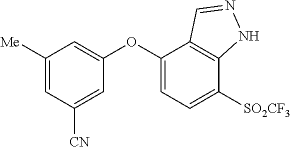

The title compound was synthesized in a similar fashion to Example 15. ¹H NMR (400 MHz, Chloroform-d) δ 8.20 (s, 1H), 7.90 (dd, J=8.4, 0.6 Hz, 1H), 7.46-7.45 (m, 1H), 7.33-7.27 (m, 2H), 6.57 (d, J=8.5 Hz, 1H), 2.49-2.42 (m, 3H). ESI MS [M+H]⁺ for C₁₆H₁₀F₃N₃O₃S, calcd 382.3, found 382.1.

### Example 29: 4-[3-Chloro-5-(trifluoromethoxy)phenoxy]-7-(trifluoromethylsulfonyl)-1H-indazole

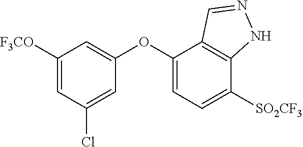

The title compound was synthesized in a similar fashion to Example 15. ¹H NMR (400 MHz, Chloroform-d) δ 8.23 (s, 1H), 7.93 (dd, J=8.4, 0.6 Hz, 1H), 7.25-7.22 (m, 1H), 7.18-7.09 (dd, J=1.8 Hz, 1H), 7.03-7.01 (m, 1H), 6.66 (d, J=8.4 Hz, 1H). ESI MS [M+H]⁺ for C₁₅H₇ClF₆N₂O₄S, calcd 461.7, found 461.1.

### Example 30: 7-(Trifluoromethylsulfonyl)-4-(3,4,5-trifluorophenoxy)-1H-indazole

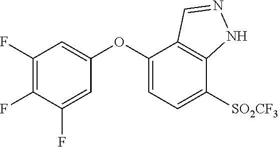

The title compound was synthesized in a similar fashion to Example 15. ¹H NMR (400 MHz, Chloroform-d) δ 8.23 (s, 1H), 7.91 (dt, J=8.4, 0.6 Hz, 1H), 6.96-6.85 (m, 2H), 6.63 (d, J=8.4 Hz, 1H). ESI MS [M+H]⁺ for C₁₄H₆F₆N₂O₃S, calcd 397.3, found 397.1.

### Example 31: 4-(3-Fluorophenoxy)-7-(trifluoromethylsulfonyl)-1H-indazole

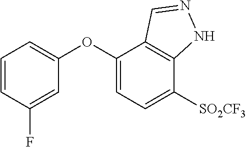

The title compound was synthesized in a similar fashion to Example 15. ¹H NMR (400 MHz, Chloroform-d) δ 8.18 (s, 1H), 7.88 (dt, J=8.4, 0.6 Hz, 1H), 7.51-7.41 (m, 1H), 7.09-7.04 (m, 1H), 7.03-7.00 (m, 1H), 6.98-6.91 (m, 1H), 6.62 (d, J=8.4 Hz, 1H). ESI MS [M+H]⁺ for C₁₄H₈F₄N₂O₃S, calcd 361.3, found 361.1.

### Example 32: 4-[3-(Trifluoromethyl)phenoxy]-7-(trifluoromethylsulfonyl)-1H-indazole

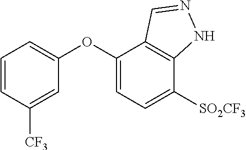

The title compound was synthesized in a similar fashion to Example 15. ¹H NMR (400 MHz, Chloroform-d) δ 8.21 (s, 1H), 7.90 (dq, J=8.5, 0.5 Hz, 1H), 7.69-7.60 (m, 2H), 7.51-7.49 (m, 1H), 7.46-7.39 (m, 1H), 6.57 (d, J=8.4 Hz, 1H). ESI MS [M+H]⁺ for C₁₅H₈F₆N₂O₃S, calcd 411.3, found 411.0.

### Example 33: 4-(4-Chloro-3-fluorophenoxy)-7-(trifluoromethylsulfonyl)-1H-indazole

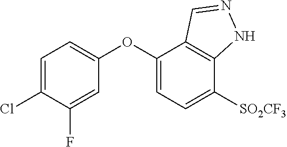

The title compound was synthesized in a similar fashion to Example 15. ¹H NMR (400 MHz, Chloroform-d) δ 8.22 (s, 1H), 7.90 (dt, J=8.4, 0.5 Hz, 1H), 7.52 (ddd, J=8.6, 8.2, 0.3 Hz, 1H), 7.09-7.03 (m, 1H), 7.01-6.97 (m, 1H), 6.62 (d, J=8.4 Hz, 1H). ESI MS [M+H]⁺ for C₁₄H₇ClF₄N₂O₃S, calcd 395.7, found 395.0.

### Example 34: 2-Fluoro-4-[[7-(trifluoromethylsulfonyl)-1H-indazol-4-yl]oxy]benzonitrile

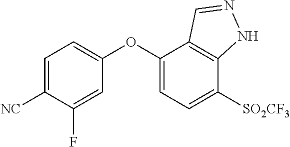

The title compound was synthesized in a similar fashion to Example 15. ¹H NMR (400 MHz, Chloroform-d) δ 8.21 (s, 1H), 8.00-7.94 (m, 1H), 7.78-7.71 (m, 1H), 7.13-7.05 (m, 2H), 6.77 (d, J=8.4 Hz, 1H). ESI MS [M+H]+ for C₁₅H₇F₄N₃O₃S, calcd 386.3, found 386.1.

### Example 35: 2-Chloro-5-[[7-(trifluoromethylsulfonyl)-1H-indazol-4-yl]oxy]benzonitrile

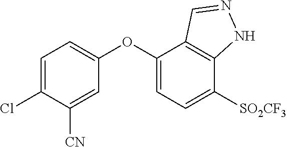

The title compound was synthesized in a similar fashion to Example 15. ¹H NMR (400 MHz, Chloroform-d) δ 8.24 (d, J=1.7 Hz, 1H), 7.92 (dt, J=8.5, 0.6 Hz, 1H), 7.67-7.61 (m, 1H), 7.55-7.53 (m, 1H), 7.43-7.40 (m, 1H), 6.59 (d, J=8.3 Hz, 1H). ESI MS [M+H]⁺ for C₁₅H₇ClF₃N₃O₃S, calcd 402.7, found 402.0.

### Example 36: 2-Fluoro-5-[[7-(trifluoromethylsulfonyl)-1H-indazol-4-yl]oxy]benzonitrile

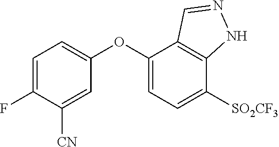

The title compound was synthesized in a similar fashion to Example 15. ¹H NMR (400 MHz, Chloroform-d) δ 8.24 (s, 1H), 7.91 (dd, J=8.4, 0.5 Hz, 1H), 7.54-7.44 (m, 2H), 7.40-7.33 (m, 1H), 6.54 (d, J=8.4 Hz, 1H). ESI MS [M+H]⁺ for C₁₅H₇F₄N₃O₃S, calcd 386.3, found 386.1.

### Example 37: 4-(3,4-Fifluorophenoxy)-7-(trifluoromethylsulfonyl)-1H-indazole

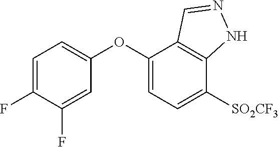

The title compound was synthesized in a similar fashion to Example 15. ¹H NMR (400 MHz, Chloroform-d) δ 9.43 (s, 1H), 8.22 (s, 1H), 7.89 (dd, J=8.5, 0.6 Hz, 1H), 7.34-7.25 (m, 1H), 7.11-7.06 (m, 1H), 6.99-6.95 (m, 1H), 6.58 (d, J=8.5 Hz, 1H). ESI MS [M+H]⁺ for C₁₄H₇F₅N₂O₃S, calcd 379.3, found 379.0.

### Example 38: 4-(3-Chloro-4-fluorophenoxy)-7-(trifluoromethylsulfonyl)-1H-indazole

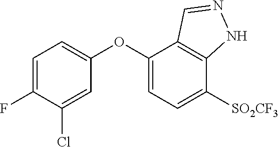

The title compound was synthesized in a similar fashion to Example 15. ¹H NMR (400 MHz, Chloroform-d) δ 8.22 (s, 1H), 7.89 (d, J=8.4 Hz, 1H), 7.32-7.25 (m, 2H), 7.13-7.09 (m, 1H), 6.56 (d, J=8.5 Hz, 1H). ESI MS [M+H]⁺ for C₁₄H₇ClF₄N₂O₃S, calcd 395.7, found 395.1.

### Example 39: 3-(Trifluoromethyl)-5-[[7-(trifluoromethylsulfonyl)-1H-indazol-4-yl]oxy]benzonitrile

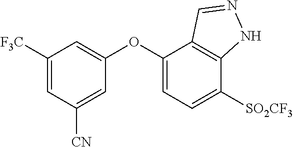

The title compound was synthesized in a similar fashion to Example 15. ¹H NMR (400 MHz, Chloroform-d) δ 8.23 (s, 1H), 7.96 (d, J=8.4 Hz, 1H), 7.88 (s, 1H), 7.73 (d, J=1.9 Hz, 1H), 7.69 (s, 1H), 6.63 (d, J=8.4 Hz, 1H). ESI MS [M+H]⁺ for C₁₆H₇F₆N₃O₃S, calcd 436.0, found 436.1.

### Example 40: 4-(3-Chloro-5-fluorophenoxy)-7-(trifluoromethylsulfonyl)-1H-indazole

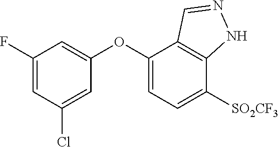

The title compound was synthesized in a similar fashion to Example 15. ¹H NMR (400 MHz, DMSO-d6) δ 13.97 (s, 1H), 8.41 (d, J=1.3 Hz, 1H), 8.04 (d, J=8.5 Hz, 1H), 7.62-7.27 (m, 3H), 6.71 (d, J=8.5 Hz, 1H). ESI MS [M+H]⁺ for C₁₄H₇ClF₄N₂O₃S, calcd 395.0, found 395.1.

### Example 41: 4-(3-Chlorophenoxy)-7-(trifluoromethylsulfonyl)-1H-indazole

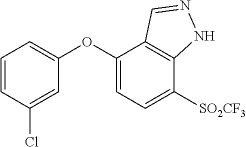

The title compound was synthesized in a similar fashion to Example 15. ¹H NMR (400 MHz, DMSO-d6) δ 13.93 (s, 1H), 8.38 (s, 1H), 8.04 (d, J=8.5 Hz, 1H), 7.64-7.52 (m, 2H), 7.45 (ddd, J=8.1, 2.0, 1.0 Hz, 1H), 7.36 (ddd, J=8.1, 2.3, 1.0 Hz, 1H), 6.60 (d, J=8.5 Hz, 1H). ESI MS [M+H]⁺ for C₁₄H₈ClF₃N₂O₃S, calcd 377.0, found 377.0.

### Example 42: 3-[[7-(Trifluoromethylsulfonyl)-1H-indazol-4-yl]oxy]benzonitrile

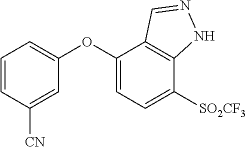

The title compound was synthesized in a similar fashion to Example 15. ¹H NMR (400 MHz, DMSO-d6) δ 13.96 (s, 1H), 8.41 (s, 1H), 8.03 (d, J=8.5 Hz, 1H), 7.99 (d, J=1.7 Hz, 1H), 7.86 (ddd, J=6.0, 2.6, 1.5 Hz, 1H), 7.77-7.70 (m, 3H), 6.62 (d, J=8.5 Hz, 2H). ESI MS [M+H]⁺ for C₁₅H₈F₃N₃O₃S, calcd 368.0, found 368.1.

### Example 43: 4-Chloro-3-[(7-trifluoromethanesulfonyl-1H-indazol-4-yl)oxy]benzonitrile

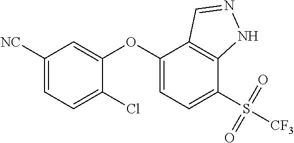 [SMILES: N#Cc1ccc(Cl)c(Oc2ccc(S(=O)(=O)C(F)(F)F)c3[nH]ncc23)c1]

The title compound was synthesized in a similar fashion to Example 15. ¹H NMR (400 MHz, DMSO-d6) δ 8.53 (s, 1H), 8.27 (dd, J=1.8, 0.5 Hz, 1H), 8.06 (d, J=8.5 Hz, 1H), 8.02-7.95 (m, 2H), 6.63 (d, J=8.4 Hz, 1H). ESI MS [M+H]+ for C₁₅H₇ClF₃N₃O₃S, calcd 402.0, found 402.0.

### Example 44: 4-Fluoro-3-[(7-trifluoromethanesulfonyl-1H-indazol-4-yl)oxy]benzonitrile

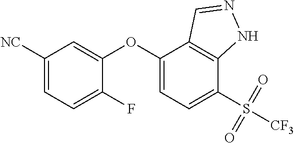 [SMILES: N#Cc1ccc(F)c(Oc2ccc(S(=O)(=O)C(F)(F)F)c3[nH]ncc23)c1]

The title compound was synthesized in a similar fashion to Example 15. ¹H NMR (400 MHz, DMSO-d6) δ 8.54 (s, 1H), 8.30 (dd, J=7.5, 2.1 Hz, 1H), 8.07 (d, J=8.5 Hz, 1H), 8.02 (ddd, J=8.6, 4.4, 2.1 Hz, 1H), 7.81 (dd, J=10.4, 8.7 Hz, 1H), 6.74 (d, J=8.5 Hz, 1H). ESI MS [M+H]⁺ for C₁₅H₇F₄N₃O₃S, calcd 386.0, found 386.0.

### Example 45: 3-Fluoro-5-[(7-trifluoromethanesulfonyl-1H-indazol-4-yl)amino] benzonitrile

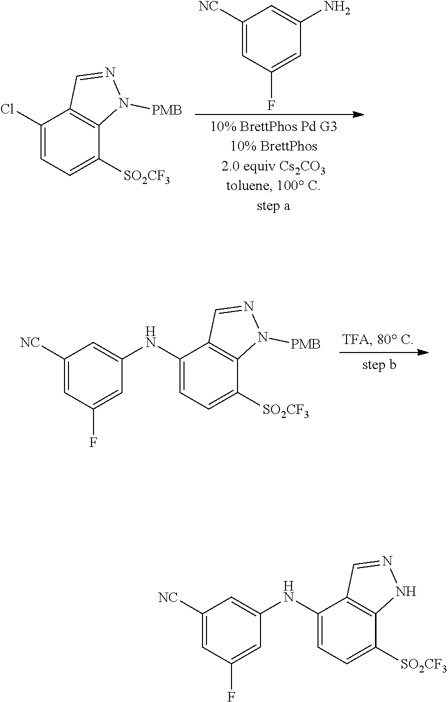

Step a. To a stirred solution of 4-chloro-1-[(4-methoxyphenyl)methyl]-7-(trifluoromethylsulfonyl)indazole (48 mg, 0.12 mmol, 1.0 equiv) in toluene (0.78 mL) was added 3-amino-5-fluoro benzonitrile (20 mg, 0.14 mmol, 1.2 equiv), BrettPhos Pd G3 (10.6 mg, 0.012 mmol, 0.1 equiv), BrettPhos (6.3 mg, 0.012 mmol, 0.1 equiv) followed by Cs₂CO₃ (77 mg, 0.23 mmol, 2.0 equiv). The mixture was degassed for 5 min while sonicating and then heated overnight with vigorous stirring. After cooling to room temperature, the reaction was partitioned between H₂O and EtOAc. The organics were washed with H₂O (3×) and brine, dried over MgSO₄, then concentrated in vacuo. The crude product was purified by column chromatography (SiO₂, hexane/EtOAc) to afford the desired aryl amine product (47 mg, 80% yield).

Step b. To a stirred solution of 3-amino-5-fluorobenzonitrile(47 mg, 0.09 mmol, 1.0 equiv) was added neat TFA (1 mL) at room temperature and then heated for 2 h. Then TFA was evaporated under reduced pressure and the resulted crude was purified using reverse phase HPLC to give the aryl amine indazole (15 mg, 42%). ¹H NMR (400 MHz, CD₃OD-d₄) δ 8.38 (s, 1H), 7.88 (d, J=8.5 Hz, 1H), 7.58 (t, J=1.9 Hz, 1H), 7.53-7.48 (m, 1H), 7.34 (dd, J=8.1, 3.7 Hz, 1H), 7.05 (d, J=8.6 Hz, 1H). ¹⁹F NMR (376 MHz, CD₃OD-d₄) δ−81.4 (s, 3F),−110.2 (s, 1F). ESI MS [M+H]⁺ for C₁₅H₈F₄N₄O₂S, calcd 385.3, found 385.1.

### Example 46: (3,5-Difluorophenyl)[7-(trifluoromethylsulfonyl)-1H-indazol-4-yl]amine

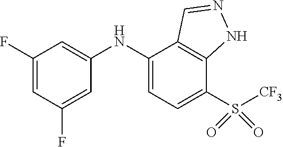 [SMILES: O=S(=O)(c1ccc(Nc2cc(F)cc(F)c2)c2cn[nH]c12)C(F)(F)F]

The title compound was synthesized in a similar fashion to Example 45. ¹H NMR (400 MHz, Chloroform-d) δ 8.13 (s, 1H), 7.84 (d, J=8.5 Hz, 1H), 6.94 (d, J=8.5 Hz, 1H), 6.91-6.81 (m, 3H), 6.72 (tt, J=2.3, 8.8 Hz, 1H). ESI MS [M+H]⁺ for C₁₄H₈F₅N₃O₂S; calcd 378.0, found 378.1.

### Example 47: (3-Chloro-5-fluorophenyl)[7-(trifluoromethylsulfonyl)-1H-indazol-4-yl]amine

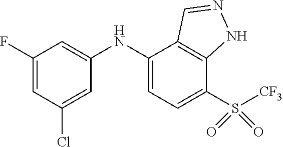 [SMILES: O=S(=O)(c1ccc(Nc2cc(F)cc(Cl)c2)c2cn[nH]c12)C(F)(F)F]

The title compound was synthesized in a similar fashion to Example 45. ¹H NMR (400 MHz, Chloroform-d) δ 8.10 (s, 1H), 7.83 (d, J=8.5 Hz, 1H), 7.14 (m, 1H), 7.04-6.93 (m, 2H), 6.91 (d, J=8.5 Hz, 1H), 6.81 (br., 1H). ESI MS [M+H]⁺ for C₁₄H₈ClF₄N₃O₂S; calcd 394.0, found 394.0.

### Example 48: 2-Fluoro-5-[7-(trifluoromethylsulfonyl)-1H-indazol-4-ylamino]benzonitrile

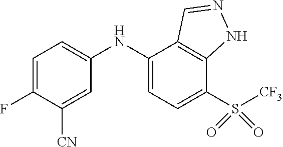 [SMILES: N#Cc1cc(Nc2ccc(S(=O)(=O)C(F)(F)F)c3[nH]ncc23)ccc1F]

The title compound was synthesized in a similar fashion to Example 45. ¹H NMR (400 MHz, Chloroform-d) δ 8.07 (s, 1H), 7.81 (d, J=8.5 Hz, 1H), 7.63-7.57 (m, 2H), 7.34 (m, 1H), 6.81 (br., 1H), 6.68 (d, J=8.5 Hz, 1H). ESI MS [M+H]+ for C₁₅H₈F₄N₄O₂S; calcd 385.0, found 385.1.

### Example 49: 6,8-Difluoro-1-(7-trifluoromethanesulfonyl-1H-indazol-4-yl)-1,2,3,4-tetrahydroquinoline

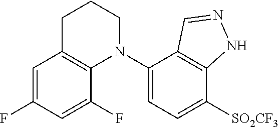

The title compound was synthesized in a similar fashion to Example 45. ¹H NMR (400 MHz, CD₃OD) δ 7.86 (d, J=8.6 Hz, 1H), 7.66 (s, 1H), 7.03-6.95 (m, 1H), 6.93-6.85 (m, 1H), 6.79 (d, J=8.6 Hz, 1H), 4.14-4.04 (m, 2H), 2.92-2.81 (m, 2H), 2.15-2.01 (m, 2H). ¹⁹F NMR (376 MHz, CD₃OD) δ-116.5,−81.4. ESI MS [M+H]⁺ for C₁₇H₁₂F₅N₃O₃S calcd 418.1, found 418.0.

### Example 50: N-[(2-Fluorophenyl)methyl]-7-(trifluoromethylsulfonyl)-1H-indazol-4-amine

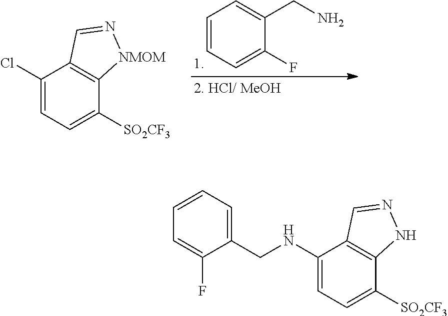

A solution of 4-chloro-1-(methoxymethyl)-7-(trifluoromethylsulfonyl)indazole (33 mg, 0.1 mmol, 1 equiv.), 2-fluorobenzylamine (3 drops), and iPr₂Net (4 drops) in ethanol (0.25 mL) was heated to reflux until the starting indazole was consumed as determined by LCMS analysis. Solvent was removed under a gentle stream of N₂. The residue was immediately dissolved in 3M HCl in methanol (˜0.5 mL) and stirred at room temperature until complete as determined by LCMS analysis. Solvent was removed under a gentle stream of N₂. The residue was purified by preparative HPLC (5-95% MeCN/H₂O+0.1% TFA), and fractions containing the product were lyophilized to give the product as a white solid. ¹H-NMR (400 MHz, DMSO-d6) δ 13.29 (bs, 1H), 8.69 (t, J=5.9 Hz, 1H), 8.49 (bs, 1H), 7.68 (d, J=8.7 Hz, 1H), 7.47-7.31 (m, 2H), 7.31-7.13 (m, 2H), 6.44 (d, J=8.8 Hz, 1H), 4.67 (d, J=5.8 Hz, 2H). ESI MS [M+H]⁺ for C₁₅H₁₁F₄N₃O₂S calcd 374.1, found 374.0.

### Example 51: N-benzyl-7-(trifluoromethylsulfonyl)-1H-indazol-4-amine

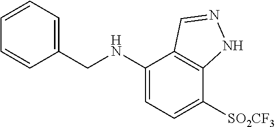

The title compound was synthesized in a similar fashion to Example 50. ¹H-NMR (400 MHz, DMSO-d6) δ 13.26 (bs, 1H), 8.79 (t, J=6.1 Hz, 1H), 8.48 (bs, 1H), 7.65 (d, J=8.7 Hz, 1H), 7.44-7.33 (m, 4H), 7.30-7.23 (m, 1H), 6.42 (d, J=8.8 Hz, 1H), 4.64 (d, J=5.9 Hz, 2H). ESI MS[M+H]⁺ for C₁₅H₁₂F₃N₃O₂S calcd 356.1, found 356.1.

### Example 52: N-[(4-fluorophenyl)methyl]-7-(trifluoromethylsulfonyl)-1H-indazol-4-amine

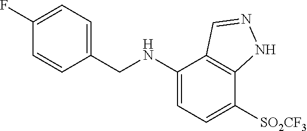

The title compound was synthesized in a similar fashion to Example 50. ¹H-NMR (400 MHz, DMSO-d6) δ 13.28 (bs, 1H), 8.76 (t, J=6.1 Hz, 1H), 8.47 (bs, 1H), 7.65 (d, J=8.7 Hz, 1H), 7.50-7.35 (m, 2H), 7.29-7.10 (m, 2H), 6.42 (d, J=8.8 Hz, 1H), 4.62 (d, J=6.0 Hz, 2H). ESI MS [M+H]⁺ for C₁₅H₁₁F₄N₃O₂S calcd 374.1, found 374.1.

### Example 53: N-[(2,4-difluorophenyl)methyl]-7-(trifluoromethylsulfonyl)-1H-indazol-4-amine

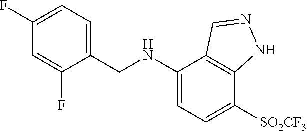

The title compound was synthesized in a similar fashion to Example 50. ¹H-NMR (400 MHz, DMSO-d6) δ 13.26 (s, 1H), 8.61 (t, J=5.8 Hz, 1H), 8.45 (bs, 1H), 7.66 (d, J=8.7 Hz, 1H), 7.44 (td, J=8.7, 6.6 Hz, 1H), 7.28 (ddd, J=10.6, 9.3, 2.6 Hz, 1H), 7.06 (tdd, J=8.5, 2.6, 1.0 Hz, 1H), 6.42 (d, J=8.8 Hz, 1H), 4.60 (d, J=5.7 Hz, 2H). ESI MS[M+H]⁺ for C₁₅H₁₀F₅N₃O₂S calcd 392.1, found 392.1.

### Example 54: N-[(2-chloro-4-fluorophenyl)methyl]-7-(trifluoromethylsulfonyl)-1H-indazol-4-amine

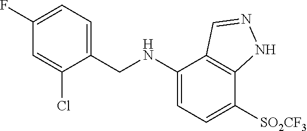

The title compound was synthesized in a similar fashion to Example 50. ¹H-NMR (400 MHz, DMSO-d6) δ 13.32 (bs, 1H), 8.67 (t, J=5.7 Hz, 1H), 8.48 (bs, 1H), 7.69 (d, J=8.7 Hz, 1H), 7.54 (dd, J=8.8, 2.6 Hz, 1H), 7.46 (dd, J=8.7, 6.2 Hz, 1H), 7.23 (td, J=8.5, 2.7 Hz, 1H), 6.37 (d, J=8.7 Hz, 1H), 4.65 (d, J=5.6 Hz, 2H). ESI MS[M+H]⁺ for C₁₅H₁₀ClF₄N₃O₂S calcd 408.0, found 408.0.

### Example 55: N-[[4-fluoro-2-(trifluoromethyl)phenyl]methyl]-7-(trifluoromethylsulfonyl)-1H-indazol-4-amine

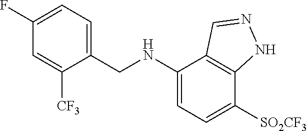

The title compound was synthesized in a similar fashion to Example 50. ¹H-NMR (400 MHz, DMSO-d6) δ 13.35 (bs, 1H), 8.73 (t, J=5.7 Hz, 1H), 8.49 (bs, 1H), 7.78-7.66 (m, 2H), 7.66-7.49 (m, 2H), 6.29 (d, J=8.7 Hz, 1H), 4.74 (d, J=5.6 Hz, 2H). ESI MS[M+H]⁺ for C₁₆H₁₀F₇N₃O₂S calcd 442.1, found 442.0.

### Example 56: N-[(3,5-dichlorophenyl)methyl]-7-(trifluoromethylsulfonyl)-1H-indazol-4-amine

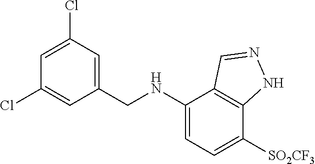

The title compound was synthesized in a similar fashion to Example 50. ¹H-NMR (400 MHz, DMSO-d6) δ 13.33 (bs, 1H), 8.73 (t, J=6.2 Hz, 1H), 8.46 (bs, 1H), 7.69 (d, J=8.7 Hz, 1H), 7.54 (t, J=1.9 Hz, 1H), 7.47 (d, J=1.9 Hz, 2H), 6.42 (d, J=8.7 Hz, 1H), 4.66 (d, J=6.1 Hz, 2H). ESI MS[M+H]⁺ for C₁₅H₁₀Cl₂F₃N₃O₂S calcd 424.0, found 424.0.

### Example 57: N-(3,3-Difluorocyclobutyl)-7-trifluoromethanesulfonyl-1H-indazol-4-amine

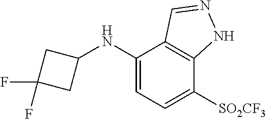

The title compound was synthesized in a similar fashion to Example 50. ¹H NMR (400 MHz, CD₃OD-d₄) δ 8.36 (s, 1H), 7.78 (d, J=8.6 Hz, 1H), 6.38 (d, J=8.8 Hz, 1H), 4.22-4.15 (m, 1H), 3.24-3.11 (m, 2H), 2.81-2.68 (m, 2H). ¹⁹F NMR (376 MHz, CD₃OD-d₄) δ-81.71 (s, 3F), −85.1 (d, J=280 Hz, 1F),−98.0 (d, J=235 Hz, 1F). ESI MS [M+H]⁺ for C₁₂H₁₀F₅N₄O₂S, calcd 356.0, found 356.1.

### Example 58: 3-Fluoro-5-[(3-methyl-7-trifluoromethanesulfonyl-1H-indazol-4-yl)oxy]benzonitrile

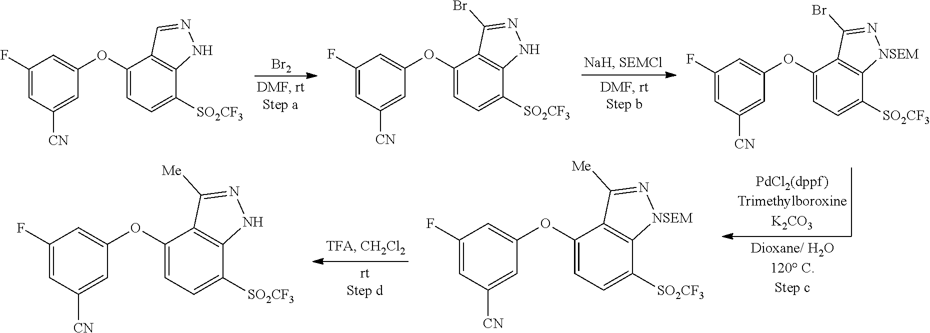

Step a. To a solution of 3-fluoro-5-[(7-trifluoromethanesulfonyl-1H-indazol-4-yl)oxy]benzonitrile (example 15) (266 mg, 0.69 mmol, 1.0 equiv.) in DMF at 0° C. was added bromine (0.106 mL, 2.06 mmol, 3.0 equiv.) dropwise. The reaction was stirred at room temperature for 3.5 hours. At this point, reaction was diluted with EtOAc (30 mL), washed with saturated aqueous Na₂S₂O₃ and brine. Layers were separated, organic layer was dried over anhydrous Na₂SO₄. Solvent was removed in vacuo to give a crude residue that was purified by column chromatography (SiO₂, gradient 0% to 30% EtOAc in hexanes) to afford 3-[(3-bromo-7-trifluoromethanesulfonyl-1H-indazol-4-yl)oxy]-5-fluorobenzonitrile (210 mg, 66% yield). ESI MS [M+H]⁺ for C₁₅H₆BrF₄N₃O₃S calcd 463.9, found 463.9.

Step b. To a solution of the product from Step a (210 mg, 0.45 mmol, 1.0 equiv.) in DMF (2.3 mL) at 0° C. was added NaH (60% dispersion in oil, 22 mg, 0.54 mmol, 1.2 equiv.) and the reaction mixture was stirred at 0° C. for 30 min. 2-(trimethylsilyl)ethoxymethyl chloride (0.10 mL, 0.58 mmol, 1.3 equiv.) was then added dropwise and the reaction was warmed to rt overnight. Reaction mixture was then cooled to 0° C., H₂O (5 mL) and EtOAc (20 mL) were added. Layers were separated and the organic layer was washed with H₂O (2×5 ml), brine and dried over anhydrous Na₂SO₄. Solvent was removed in vacuo to give a crude residue that was purified by column chromatography (SiO₂, gradient 0% to 20% EtOAc in hexanes) to afford the desired product (280 mg, quant. yield).

Step c. To a solution of the product from Step b (130 mg, 0.22 mmol, 1.0 equiv.) in degassed dioxane (1.0 mL) and H₂O (0.2 mL) under nitrogen was added trimethylboroxine (0.040 mL, 0.28 mmol, 1.3 equiv.), K₂CO₃ (90 mg, 0.65 mmol, 3.0 equiv.) and PdCl₂dppf (16 mg, 0.022 mmol, 0.1 equiv.). The reaction vessel was evacuated and refilled with nitrogen. This process was repeated twice, and the reaction was heated to 120° C. for 16 hours. At this point, the reaction was filtered over Celite® and the filter cake was washed with EtOAc. Solvent was subsequently removed in vacuo to give a crude residue that was purified by column chromatography (SiO₂, gradient 0% to 20% EtOAc in hexanes) to afford the desired product (20 mg, 17% yield).

Step d. To a solution of the product from Step c (20 mg, 0.037 mmol) was added 4N HCl in dioxane (2 mL) and reaction was stirred at rt for 1 hour. Solvent was subsequently removed in vacuo and the crude residue was purified by reverse phase HPLC (MeCN/H₂O) to provide 3-fluoro-5-[(3-methyl-7-trifluoromethanesulfonyl-1H-indazol-4-yl)oxy]benzonitrile (5.0 mg, 34% yield). ¹H NMR (400 MHz, CD₃OD) δ 8.08-7.93 (m, 1H), 7.67-7.58 (m, 2H), 7.57-7.53 (m, 1H), 6.64 (d, J=8.5 Hz, 1H), 2.73 (s, 3H). ¹⁹F NMR (376 MHz, CDCl₃) δ-108.2,−81.2. ESI MS [M+H]⁺ for C₁₆H₉F₄N₃O₃S calcd 400.0, found 400.0.

### Example 59: 4-[(3-Chlorophenyl)methyl]-7-trifluoromethanesulfonyl-1H-indazole

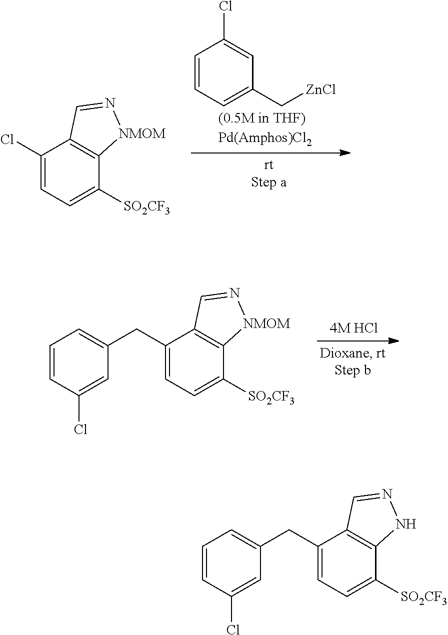

Step a. To a mixture of 4-chloro-1-(methoxymethyl)-7-(trifluoromethylsulfonyl)indazole (200 mg, 0.609 mmol, 1.0 equiv.) and Pd(Amphos)Cl₂ (43.1 mg, 0.0609 mmol, 0.10 equiv.) under nitrogen was added 3-chlorobenzylzinc chloride (0.5M in THF, 1.46 mL, 1.2 equiv.). The reaction was stirred at rt for 1 hour. At this point, reaction was quenched with saturated aqueous NH₄Cl and extracted into EtOAc (2×20 mL). Layers were separated, organic layer was washed with brine and dried over anhydrous Na₂SO₄. Solvent was removed in vacuo to give a crude residue that was purified by column chromatography (SiO₂, gradient 0% to 20% EtOAc in hexanes) to afford 4-[(3-chlorophenyl)methyl]-1-(methoxymethyl)-7-(trifluoromethanesulfonyl)-1H-indazole (200 mg, 78% yield). ESI MS [M+H]⁺ for C₁₇H₁₄ClF₃N₂O₃S calcd 419.0, found 419.0.

Step b. To a solution of 4-[(3-chlorophenyl)methyl]-1-(methoxymethyl)-7-(trifluoromethanesulfonyl)-1H-indazole from Step a (40 mg, 0.096 mmol) was added 4M HCl in dioxane (3 mL) and reaction was stirred at rt for 1 hour. Solvent was subsequently removed in vacuo and the crude residue was purified by reverse phase HPLC (MeCN/H₂O) to provide 4-[(3-chlorophenyl)methyl]-7-trifluoromethanesulfonyl-1H-indazole (27 mg, 76% yield). ¹H NMR (400 MHz, CDCl₃) δ 8.21 (s, 1H), 7.97 (dd, J=7.7, 0.5 Hz, 1H), 7.29-7.26 (m, 2H), 7.25-7.22 (m, 1H), 7.20-7.17 (m, 1H), 7.14-7.17 (m, 1H), 4.40 (s, 2H). ¹⁹F NMR (376 MHz, CDCl₃) δ-79.0. ESI MS [M+H]⁺ for C₁₅H₁₀ClF₃N₂O₂S calcd 375.0, found 375.0.

### Example 60: 4-(3-Cyano-5-fluorophenoxy)-7-trifluoromethanesulfonyl-1H-indazole-3-carbonitrile

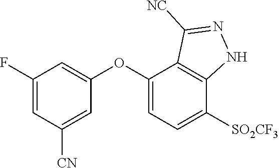

The title compound was synthesized in a similar fashion to Example 10. ¹H NMR (400 MHz, CD₃OD) δ 8.18 (d, J=8.5 Hz, 1H), 7.72-7.60 (m, 3H), 6.91 (d, J=8.5 Hz, 1H). ¹⁹F NMR (376 MHz, CD₃OD) δ-108.0,−80.9. ESI MS [M+H]⁺ for C₁₆H₆F₄N₄O₃S calcd 411.0, found 411.0.

### Example 61: 3-[(3-Chloro-7-trifluoromethanesulfonyl-1H-indazol-4-yl)oxy]-5-fluorobenzonitrile

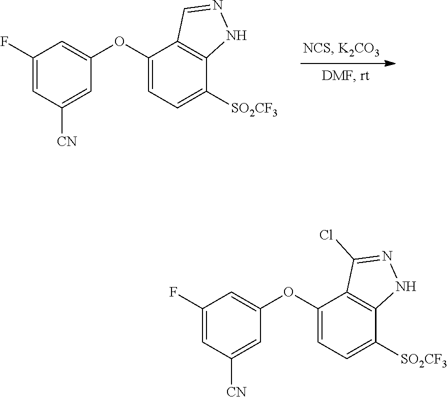

To a solution of 3-fluoro-5-[(7-trifluoromethanesulfonyl-1H-indazol-4-yl)oxy]benzonitrile (example 15) (80 mg, 0.20 mmol, 1.0 equiv.) in DMF (2 mL) was added K₂CO₃ (55.3 mg, 0.40 mmol, 2.0 equiv.) and N-chlorosuccinimide (53.4 mg, 0.40 mmol, 2.0 equiv.) and the reaction mixture was stirred at rt for 3 hours. Solvent was subsequently removed in vacuo to give a crude residue that was purified by column chromatography (Sift, gradient 0% to 20% EtOAc in hexanes) to afford 3-[(3-chloro-7-trifluoromethanesulfonyl-1H-indazol-4-yl)oxy]-5-fluorobenzonitrile (27 mg, 32% yield). ¹H NMR (400 MHz, CDCl₃) δ 10.87 (s, 1H), 7.96 (d, J=8.5 Hz, 1H), 7.47-7.32 (m, 2H), 7.25-7.22 (m, 1H), 6.63 (d, J=8.4 Hz, 1H). ¹⁹F NMR (376 MHz, CDCl₃) δ-104.7,−78.9. ESI MS [M−H]⁻ for C₁₅H₆ClF₄N₃O₃S calcd 417.9, found 418.0.

### Example 62: m-[3-Chloro-7-(trifluoromethylsulfonyl)-1H-indazol-4-yloxy]benzonitrile

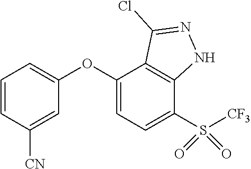 [SMILES: N#Cc1cccc(Oc2ccc(S(=O)(=O)C(F)(F)F)c3[nH]nc(Cl)c23)c1]

The title compound was synthesized in a similar fashion to Example 61. ¹H NMR (400 MHz, Chloroform-d) δ 7.89 (d, J=8.5 Hz, 1H), 7.70-7.59 (m, 2H), 7.56-7.52 (m, 1H), 7.48 (m, 1H), 6.50 (d, J=8.5 Hz, 1H). ESI MS [M+H]⁺ for C₁₅H₇ClF₃N₃O₃S; calcd 401.9, found 402.0.

### Example 63: 3-Chloro-4-(m-chlorophenoxy)-7-(trifluoromethylsulfonyl)-1H-indazole

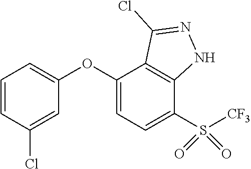 [SMILES: O=S(=O)(c1ccc(Oc2cccc(Cl)c2)c2c(Cl)n[nH]c12)C(F)(F)F]

The title compound was synthesized in a similar fashion to Example 61. ¹H NMR (400 MHz, Chloroform-d) δ 7.86 (d, J=8.5 Hz, 1H), 7.44 (t, J=8.0 Hz, 1H), 7.36-7.32 (m, 1H), 7.14-7.11 (m, 1H), 6.52 (d, J=8.5 Hz, 1H). ESI MS [M+H]+ for C₁₄H₇Cl₂F₃N₂O₃S; calcd 410.9, found 412.0.

### Example 64: 3-Fluoro-5-[(5-fluoro-7-trifluoromethanesulfonyl-1H-indazol-4-yl)oxy]benzonitrile

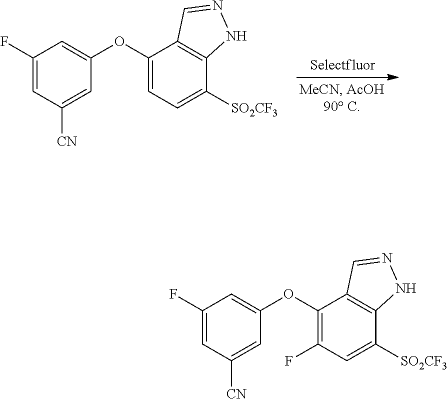

To a solution of 3-fluoro-5-[(7-trifluoromethanesulfonyl-1H-indazol-4-yl)oxy]benzonitrile (example 15) (50 mg, 0.129 mmol) in acetonitrile (0.4 mL) and acetic acid (0.016 mL) was added Selectfluor (109 mg, 0.301 mmol) and the reaction mixture was heated to 90° C. for 16 hours. Reaction mixture was concentrated in vacuo and the crude residue was purified by reverse phase HPLC (MeCN/H₂O) to provide 3-fluoro-5-[(5-fluoro-7-trifluoromethanesulfonyl-1H-indazol-4-yl)oxy]benzonitrile (5 mg, 10% yield). ¹H NMR (400 MHz, CD₃OD) δ 8.20-8.15 (m, 1H), 8.01 (s, 1H), 7.52-7.46 (m, 2H), 7.44-7.37 (m, 1H). ¹⁹F NMR (376 MHz, CD₃OD) δ-140.3,−108.7,−80.8. ESI MS [M+H]⁺ for C₁₅H₆F₅N₃O₃S calcd 404.0, found 404.0.

### Example 65: 3-[(6-Chloro-1H-indazol-4-yl)amino]-5-florobenzonitrile

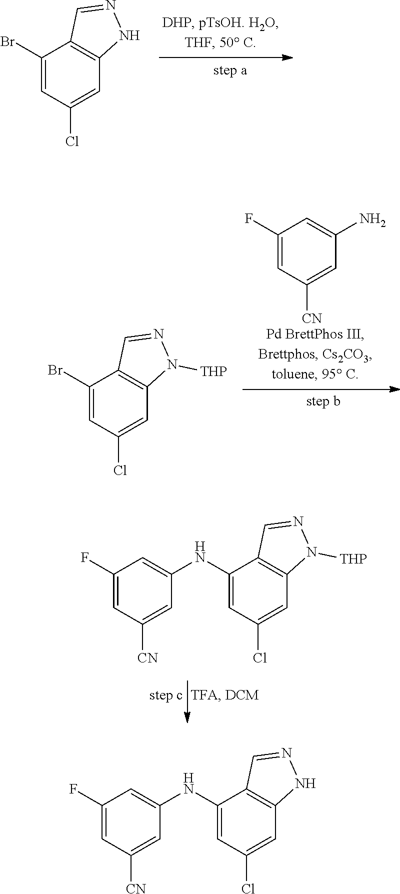

Step a. To a flask containing 4-bromo-6-chloro-1H-indazole (2.00 g, 8.69 mmol, 1.0 equiv.) was added 3,4-dihydro-2H-pyran (2.18 g, 26.1 mmol, 3.0 equiv.) and THF (30 mL). pTsOH.H₂O (0.330 g, 1.73 mmol, 20 mol %) was added and the reaction mixture was warmed to 50° C. and stirred for 4 h. The reaction was partitioned between sat. aq. NaHCO₃ solution and EtOAc. The aqueous layer was separated and back extracted with additional EtOAc. The organic layers were combined and dried over MgSO₄. Concentration under reduced pressure and purification by flash chromatography (SiO₂, hexanes→50% EtOAc) furnished the THP protected indazole as an orange oil (1.14 g, 3.6 mmol, 42%).

Step b. To a vial containing the product from Step a (100 mg, 0.317 mmol, 1.0 equiv.) was added toluene (2 mL), followed by 3-amino-5-fluoro-benzonitrile (40 mg, 0.317 mmol, 1.0 equiv.), Pd BrettPhos III (29 mg, 0.032 mmol, 10 mol %), BrettPhos (17 mg, 0.032 mmol, 10 mol %), and Cs₂CO₃ (0.210 g, 0.634 mmol, 2.0 equiv.). The reaction mixture was purged with nitrogen, capped, heated to 95° C. and stirred for 15 h. Concentration under reduced pressure and purification by flash chromatography (SiO₂, hexanes→40% EtOAc) furnished the indazole product (81 mg, 0.218 mmol, 69%, ESI MS [M+Na]⁺ for C₁₉H₁₆ClFN₄O, calcd 393.10, found 393.0).

Step c. The product from Step b (81 mg, 0.218 mmol) was dissolved in DCM (1 mL). TFA (0.5 mL) was added and the reaction was stirred at room temperature for 1.5 h. The reaction was partitioned between sat. aq. NaHCO₃ solution and DCM. The aqueous layer was separated and back extracted with additional DCM. The organic layers were combined and dried over MgSO₄. Concentration under reduced pressure and purification by flash chromatography (SiO₂, hexanes→80% EtOAc), followed by preparative reverse phase HPLC (20 to 80% gradient of acetonitrile and water with 0.1% TFA) furnished the title compound as a white powder. ¹H NMR (400 MHz, DMSO-d6) δ 9.21 (s, 1H), 8.10 (d, J=1.0 Hz, 1H), 7.41-7.38 (m, 1H), 7.35-7.27 (m, 2H), 7.19 (dd, J=1.5, 1.0 Hz, 1H), 6.87 (d, J=1.5 Hz, 1H). ESI MS [M+H]⁺ for C₁₄H₈ClFN₄, calcd 287.0, found 287.0.

### Example 66: 3-Fluoro-5-({1H-pyrazolo[3,4-c]pyridin-4-yl}amino)benzonitrile

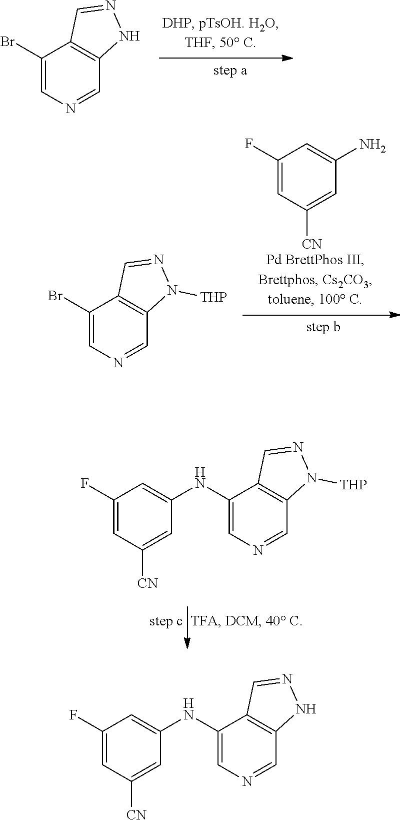

Step a. To a flask containing 4-bromo-1H-pyrazolo[3,4-c]pyridine (1.00 g, 5.05 mmol, 1.0 equiv.) was added 3,4-dihydro-2H-pyran (0.90 mL, 10.1 mmol, 2.0 equiv.) and THF (25 mL). pTsOH.H₂O (0.140 g, 0.758 mmol, 15 mol %) was added and the reaction mixture was warmed to 50° C. and stirred for 16 h. The reaction was partitioned between sat. aq. NaHCO₃ solution and EtOAc. The aqueous layer was separated and back extracted with additional EtOAc. The organic layers were combined and dried over MgSO₄. Concentration under reduced pressure and purification by flash chromatography (SiO₂, hexanes→50% EtOAc) furnished the THP protected indazole as a yellow oil (0.98 g, 3.6 mmol, 69%, ESI MS [M+H]⁺ for C₁₁H₁₂BrN₃O, calcd 282.0, found 282.0).

Step b. To a vial containing the product from Step a (600 mg, 2.13 mmol, 1.0 equiv.) was added toluene (10 mL), followed by 3-amino-5-fluoro-benzonitrile (434 mg, 3.20 mmol, 1.5 equiv.), Pd BrettPhos III (154 mg, 0.170 mmol, 8 mol %), BrettPhos (91 mg, 0.170 mmol, 8 mol %), and Cs₂CO₃ (1.40 g, 4.26 mmol, 2.0 equiv.). The reaction mixture was purged with nitrogen, capped, heated to 100° C. and stirred for 15 h. Concentration under reduced pressure and purification by flash chromatography (SiO₂, hexanes→80% EtOAc) furnished the indazole product (266 mg, 0.789 mmol, 37% ESI MS [M+H]⁺ for C₁₅H₁₆FN₅O, calcd 338.1, found 338.2).

Step c. The product from Step b (50 mg, 0.148 mmol) was dissolved in DCM (1 mL). TFA (1 mL) was added and the reaction was stirred at room temperature for 1.5 h. The reaction was partitioned between sat. aq. NaHCO₃ solution and DCM. The aqueous layer was separated and back extracted with additional DCM. The organic layers were combined and dried over MgSO₄. Concentration under reduced pressure and purification by flash chromatography (SiO₂, hexanes→80% EtOAc), followed by preparative reverse phase HPLC (20 to 80% gradient of acetonitrile and water with 0.1% TFA) furnished the title compound as a yellow solid. ¹H NMR (400 MHz, DMSO-d6) δ 9.69 (s, 1H), 9.00 (s, 1H), 8.49 (s, 1H), 8.18 (s, 1H), 7.54 (s, 1H), 7.50-7.39 (m, 2H). ESI MS [M+H]⁺ for C₁₃H₈FN₅, calcd 254.1, found 254.1.

### Example 67: 3-Fluoro-5-({1H-pyrazolo[3,4-d]pyrimidin-4-yl}amino)benzonitrile

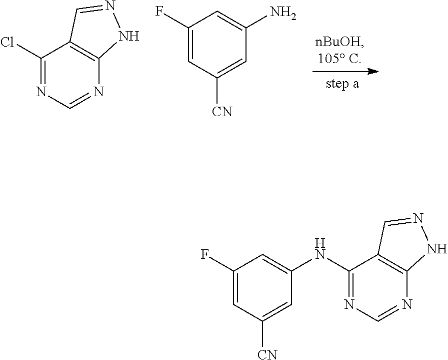

A vial containing 4-chloro-1H-pyrazolo[3,4-d]pyrimidine (80 mg, 0.519 mmol, 1.0 equiv.), 3-amino-5-fluoro-benzonitrile (140 mg, 1.04 mmol, 2.0 equiv.) and nBuOH (2 mL) was heated to 105° C. Concentration under reduced pressure and purification by reverse phase HPLC (20 to 80% gradient of acetonitrile and water with 0.1% TFA) furnished the title compound as a white powder. ¹H NMR (400 MHz, DMSO-d6) δ 10.49 (s, 1H), 8.55 (s, 1H), 8.36 (s, 1H), 8.26-8.17 (m, 2H), 7.58-7.52 (m, 1H). ESI MS [M+H]⁺ for C₁₂H₇FN₆, calcd 255.1, found 255.1.

### Example 68: 3-Fluoro-5-{[7-(trifluoromethyl)-1H-pyrazolo[3,4-c]pyridin-4-yl]oxy}benzonitrile

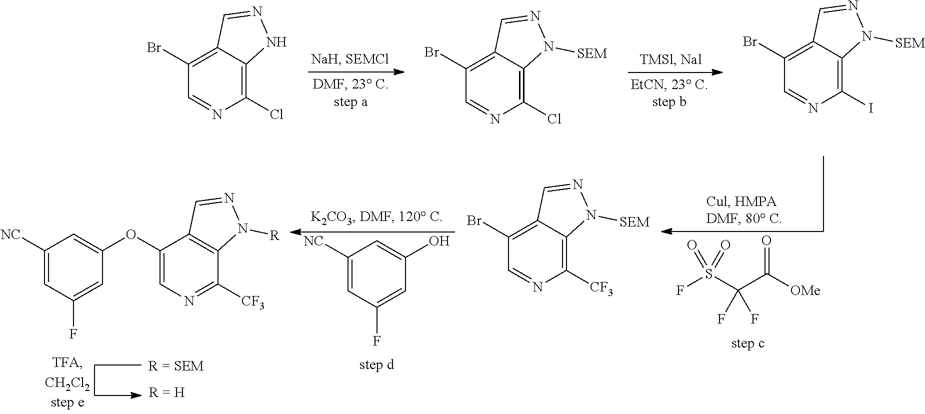

Step a. To a solution of 4-bromo-7-chloro-1H-pyrazolo[3,4-c]pyridine (1.20 g, 5.16 mmol, 1.0 equiv.) in DMF (25 mL) was added NaH (0.25 g, 6.19 mmol, 1.2 equiv., 60%) in portions. The reaction mixture was stirred at 0° C. for 30 minutes and then 2-(trimethylsilyl)ethoxymethyl chloride (1.10 mL, 6.19 mmol, 1.2 equiv.) was added dropwise over 10 minutes. The resulting mixture was stirred for 2 h at room temperature. The reaction was quenched with aqueous NH₄Cl solution and partitioned between EtOAc and water. The organic phase was washed with brine, dried over Na₂SO₄ and evaporated under reduced pressure. The resulting residue was purified by chromatography on silica gel (0 to 25% gradient EtOAc in Hexane) to obtain the product as a colorless oil (1.83 g, 98%). ESI MS [M+H]⁺ for C₁₂H₁₇BrClN₃OSi, calcd 362.0, found 362.0.

Step b. The product from Step a (1.83 g, 5.05 mmol, 1.0 equiv.) was dissolved in propionitrile (34 ml) and iodotrimethylsilane (0.72 ml, 5.05 mmol, 1.0 equiv.) and sodium iodide (2.26 g, 15.14 mmol, 3.0 equiv.) were added sequentially. The mixture was stirred at room temperature for 1h and the solvent was evaporated. The resulting solid was dissolved in H₂O and the pH was adjusted to basic with 2M NaOH. Dichloromethane was then added, the organic phase was separated, dried over Na₂SO₄, filtered and concentrated to give the desired product as an orange solid (2.0 g, 87%), which was used directly in the next step without further purification. ESI MS [M+H]⁺ for C₁₂H₁₇BrIN₃OSi, calcd 454.0, found 454.0.

Step c. The product of Step b (2.0 g, 4.40 mmol, 1.0 equiv.) was dissolved in anhydrous DMF (12 mL) in a round-bottom flask under an atmosphere of nitrogen. CuI (1.23 g, 6.16 mmol, 1.4 equiv.), methyl 2,2-difluoro-2-(fluorosulfonyl)acetate (2.8 ml, 22.0 mmol, 5.0 equiv.) and HMPA (3.8 ml, 22.0 mmol, 5.0 equiv.) were then added sequentially. The reaction mixture was stirred at 80° C. for 16h. Upon completion, the solvent was evaporated, and the residue was dissolved in EtOAc and washed with 1N NH₄C₁ three times. The organic layer was separated, dried over anhydrous Na₂SO₄, filtered and evaporated under reduced pressure. The resulting residue was purified by chromatography on silica gel (0 to 12% gradient EtOAc in Hexane) to obtain the product as a yellow solid (450 mg, 25%). ESI MS [M+H]⁺ for C₁₃H₁₇BrF₃N₃OSi, calcd 396.0, found 396.0.

Step d. The product from Step c (120 mg, 0.30 mmol, 1.0 equiv.) was dissolved in DMF (3.0 mL) and 3-hydroxy-5-fluoro-benzonitrile (83 mg, 0.604 mmol, 2.0 equiv.) was added followed by K₂CO₃ (84 mg, 0.604 mmol, 2.0 equiv.). The reaction was stirred at 120° C. for 5h. The reaction mixture was diluted with EtOAc and then washed with a sat. sol. NaCl. The organic layer was separated, dried over anhydrous Na₂SO₄, filtered and evaporated under reduced pressure. The resulting residue was purified by chromatography on silica gel (0 to 15% gradient EtOAc in Hexane) to obtain the product as a yellow solid (28 mg, 20%). ESI MS [M+H]⁺ for C₂₀H₂₀F₄N₄O₂Si, calcd 453.0, found 453.0.

Step e. The product of Step d (28 mg, 0.062 mmol) was dissolved in a mixture of trifluoroacetic acid and DCM (1:1, 3.0 mL) and the reaction mixture was stirred for 1h at room temperature. The mixture was then concentrated in vacuum, the residue was dissolved in DMSO (2 ml), and the product was purified by reverse phase HPLC (20 to 80% gradient of acetonitrile and water with 0.1% TFA) to afford the title compound as a pale slightly yellow solid. ¹H NMR (400 MHz, DMSO-d6) δ: 8.27 (d, J=1.3 Hz, 1H), 8.12 (s, 1H), 7.80 (ddd, J=8.4, 2.4, 1.3 Hz, 1H), 7.74 (s, 1H), 7.69 (dt, J=9.9, 2.3 Hz, 1H), 6.55 (s, 1H). ESI MS [M+H]⁺ for C₁₄H₆F₄N₄O, calcd 323.0, found 323.0.

### Example 69: 4-(3-Chloro-5-fluorophenoxy)-7-(trifluoromethyl)-1H-pyrazolo[3,4-c]pyridine

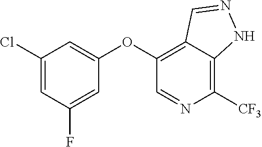 [SMILES: Fc1cc(Cl)cc(Oc2cnc(C(F)(F)F)c3[nH]ncc23)c1]

The title compound was synthesized in a similar fashion to Example 68. ¹H NMR (400 MHz, DMSO-d6) δ: 8.25 (d, J=1.3 Hz, 1H), 8.08 (s, 1H), 7.39 (ddd, J=8.7, 2.3, 1.8 Hz, 1H), 7.31 (s, 1H), 7.28 (dt, J=9.8, 2.2 Hz, 1H). ESI MS [M+H]⁺ for C₁₃H₆ClF₄N₃O, calcd 332.0, found 332.0.

### Example 70: 3-Fluoro-5-[4-(trifluoromethylsulfonyl)-1H-indazol-7-yloxy]benzonitrile

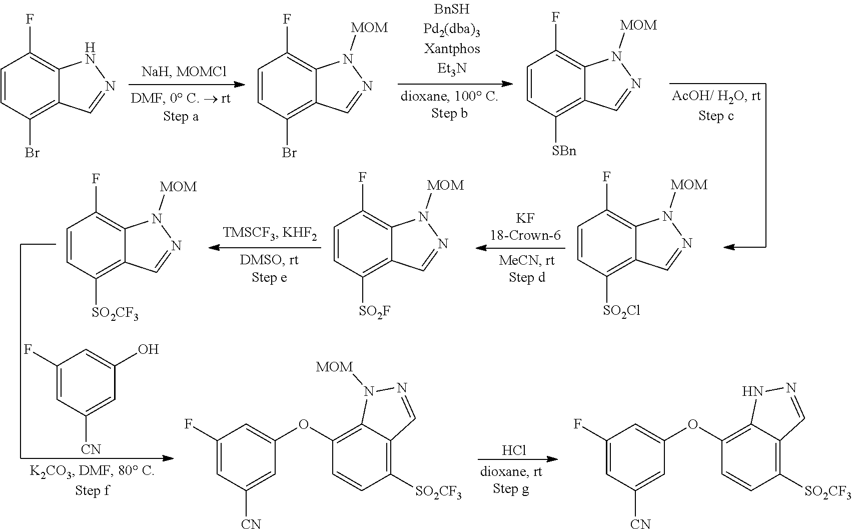

Step a. To a solution of 4-bromo-7-fluoro-1H-indazole (5 g, 23 mmol, 1.0 equiv.) in DMF (60 mL) at 0° C. was added sodium hydride (29 mmol, 1.25 equiv.). After stirring for 30 min at 0° C., chloromethyl methyl ether (2.0 mL, 26 mmol, 1.1 equiv.) was added dropwise. After addition, the ice bath was removed, and the reaction was stirred for 16 hours at room temperature. The reaction mixture was quenched with a saturated aqueous NH₄Cl solution and the aqueous phase was extracted with EtOAc (3×30 mL). The combined organic layers were washed with brine, dried over Na₂SO₄, filtered and concentrated by rotary evaporation. The crude product was purified by silica gel column chromatography (0 to 30% EtOAc in hexanes) to afford the desired product (4.3 g, 70% yield). ESI MS [M+H]⁺ for C₉H₈BrFN₂O, calcd 259.0, found 259.0.

Step b. The product from Step a (4.2 g, 16.3 mmol, 1.0 equiv.) was dissolved in dry dioxane (54 mL) and the stirred solution was evacuated and refilled with nitrogen three times. To this solution benzyl mercaptan (2.3 mL, 19.6 mmol, 1.2 equiv.), Et₃N (6.8 mL, 49 mmol, 3.0 equiv.), Xanthos (940 mg, 1.63 mmol, 0.1 equiv.) and Pd₂(dba)₃ (750 mg, 0.82 mmol, 0.05 equiv.) were added, after which the resulting mixture was evacuated and refilled with nitrogen three times. After stirring for 90 min at 100° C., the reaction mixture was quenched with water and the aqueous phase was extracted with EtOAc (3×50 mL). The combined organic layers were washed with brine, dried over Na₂SO₄, filtered and concentrated by rotary evaporation. The crude product was purified by silica gel column chromatography (0 to 20% EtOAc in hexanes) to afford the desired product (5.3 g, 93% yield). ESI MS [M+H]⁺ for C₁₆H₁₅FN₂OS, calcd 303.1, found 303.0.

Step c. The product from Step b (4.5 g, 15 mmol, 1.0 equiv.) was dissolved in AcOH/H₂O (9:1, 50 mL). To this solution NCS (7.9 g, 60 mmol, 4.0 equiv.) was added in ˜1 g portions over 5 min. The resulting mixture was stirred for 30 min at room temperature and monitored by LC-MS. After completion of the reaction, the mixture was poured into water and treated with NaHCO₃. The aqueous phase was extracted with EtOAc (3×50 mL). The combined organic layers were washed with brine, dried over Na₂SO₄, filtered and concentrated by rotary evaporation. The crude product was purified by silica gel column chromatography (0 to 30% EtOAc in hexanes) to afford the desired product as a white solid (3.6 g, 87% yield). ESI MS [M+H]⁺ for C₉H₈ClFN₂O₃S, calcd 279.0, found 279.0.

Step d. The product from Step c (3.6 g, 13 mmol, 1.0 equiv.) was dissolved in MeCN (13 mL). To this solution 18-crown-6 (0.18 g, 0.7 mmol, 0.05 equiv.) and potassium fluoride (0.32 g, 52 mmol, 4.0 equiv.) were added. The resulting mixture was stirred for 1 hour at room temperature and monitored by LC-MS. The reaction mixture was quenched with water and the aqueous phase was extracted with EtOAc (3×50 mL). The combined organic layers were washed with brine, dried over Na₂SO₄, filtered and concentrated by rotary evaporation. The crude product was purified by silica gel column chromatography (0 to 20% EtOAc in hexanes) to afford the desired product as a white solid (2.7 g, 80% yield). ESI MS [M+H]⁺ for C₉H₈F₂N₂O₃S, calcd 263.0, found 263.0.

Step e. The product from Step d (2.7 g, 10.3 mmol, 1.0 equiv.) was dissolved in dry DMSO (20 mL) and the stirred solution was evacuated and refilled with nitrogen three times. To this solution potassium bifluoride (0.24 g, 3.1 mmol, 0.3 equiv.) and trifluoromethyltrimethylsilane (3.0 mL, 20.6 mmol, 2.0 equiv.) were added sequentially. The resulting mixture was stirred for 15 min at room temperature and monitored by LC-MS. The reaction mixture was quenched with water and the aqueous phase was extracted with EtOAc (3×50 mL). The combined organic layers were washed with brine, dried over Na₂SO₄, filtered and concentrated by rotary evaporation. The crude product was purified by silica gel column chromatography (0 to 20% EtOAc in hexanes) to afford the desired product as a white solid (2.0 g, 63% yield). ESI MS [M+H]⁺ for C₁₀H8F₄N₂O₃S, calcd 313.0, found 313.0.

Step f. The product from Step e (0.18 g, 0.6 mmol, 1.0 equiv.) was dissolved in dry DMF (1.2 mL). To this solution 3-fluoro-5-hydroxybenzonitrile (0.16 g, 1.2 mmol, 2.0 equiv.) and K₂CO₃ (0.16 g, 1.2 mmol, 2.0 equiv.) were added and the resulting mixture was heated to 80° C. After 30 min, the mixture was treated with water and the aqueous phase was extracted with EtOAc. The combined organic layers were washed with brine, dried over Na₂SO₄, filtered and concentrated by rotary evaporation. The crude product was purified by silica gel column chromatography (0 to 30% EtOAc in hexanes) to afford the desired product as a white solid (0.24 g, 96% yield). ESI MS [M+H]⁺ for C₁₇H₁₁F₄N₃O₄S, calcd 430.0, found 430.0.

Step g. To the product from Step f (0.24 g, 0.56 mmol) was added 4 N HCl in dioxane (6 mL) and the mixture was stirred at room temperature. After 15 hours, the reaction mixture was concentrated by rotary evaporation. The crude product was purified by silica gel column chromatography (0 to 50% EtOAc in hexanes) to afford the desired product as a white solid (0.12 g, 54% yield). ¹H NMR (400 MHz, CDCl₃) δ 8.55 (s, 1H), 7.91 (d, J=8.2 Hz, 1H), 7.40-7.35 (m, 2H), 7.26-7.23 (m, 1H), 6.91 (d, J=8.2 Hz, 1H). ESI MS [M+H]⁺ for C₁₅H₇F₄N₃O₃S, calcd 386.02, found 386.0.

### Analytical Methods:

LC: Agilent 1100 series; Mass spectrometer: Agilent G6120BA, single quad

LC-MS method: Agilent Zorbax Eclipse Plus C18, 4.6×100 mm, 3.5 μM, 35° C., 1.5 mL/min flow rate, a 2.5 min gradient of 0% to 100% B with 0.5 min wash at 100% B; A=0.1% of formic acid/5% acetonitrile/94.9% water; B=0.1% of formic acid/5% water/94.9% acetonitrile

Flash column: ISCO Rf+

Reverse phase HPLC: ISCO-EZ or Agilent 1260; Column: Kinetex 5 μm EVO C18 100 A; 250×21.2 mm (Phenomenex)

### Biological Examples

### Generation of HIF-2α Luciferase 786-0 Cell Line:

Stable cell lines were generated by transducing 786-O cells (ATCC, CRL-1932) with Cignal Lenti HIF Luc Reporter lentivirus (CLS-007L, Qiagen) according to the manufacturer's guidelines. In brief, 0.3×106 786-0 cells were transduced with lentivirus at a Multiplicity of Infection (MOI) of 25 for 24 hours. After transduction, cells were replenished with fresh RPMI 1640 Medium (Cat. No. 11875085, Thermo Fisher,) supplemented with 10% FBS (Cat. No. A3160502, Gibco), 2 mM GlutaMax (Cat. No. 35050-061, Invitrogen) and 100 units of penicillin and 100 μg of streptomycin/mL (Cat. No 15070063, Thermo Fisher) for another 24 hours. Antibiotic selection was performed in cell media containing 4 μg/mL of Puromycin. After 7 days of antibiotic selection, stable pools of surviving cells were expanded and used in a luciferase reporter assay.

### HIF-2α Luciferase Reporter Assay:

On day one, 20 uL of HIF-Luc-786-0 cells in OptiMem (Cat. No. 31985088, Thermo Fisher) were seeded into each well of a 384 well white opaque plate (Corning 3570) and incubated at 37° C. and 5% CO₂. Twenty microliters of 2× test compounds in OptiMem were added to cells after 4 hours of incubation. Final assay conditions comprised 20,000 cells per well in 1% DMSO with test compound concentrations ranging from 50 uM to 0 uM. After 20 hours incubation at 37° C. and 5% CO₂, luciferase activity was determined using ONE-Glo Luciferase Assay Reagent (E6110, Promega) following the manufacture's recommended procedure. Briefly, 40 uL of ONE-Glo luciferase reagents were added to each well and luciferase signals were measured using an Envision 2102 Multilabel Reader. Percentage maximum activity in each test well was calculated based on DMSO (maximum activity) and no cell control wells (baseline activity). The IC50 values of the test compounds were determined from compound dose response curves fitted using a standard four parameter fit equation.

### HIF-2α Scintillation Proximity Assay (SPA):

Tritium labeled compound N-(3-chlorophenyl)-4-nitro-2,1,3-benzoxadiazol-5-amine was obtained from American Radiolabeled Chemicals Inc. and copper chelate PVT SPA beads were from PerkinElmer (Cat #RPNQ0095). Histidine tagged HIF-2α protein containing PAS-B domain (240-350) was prepared and purified in house.

Compounds solubilized in DMSO were dispensed into a white 384-well polystyrene non-binding flat clear bottom plate (Greiner Bio-One, Cat #781903) using an HP D300 dispenser. Ten microliters of HIS-tagged HIF-2α protein in buffer (25 mM Tris-HCl, pH 7.4, 150 mM NaCl, 0.15% BSA and 0.001% Tween 20) was added to the compound wells and allowed to incubate for 1 hour at room temperature. Ten microliters of SPA bead mix were added to the wells and incubated for an additional 45 minutes, followed by 10 ul of ³H-tracer solution. Final assay conditions comprised 50 nM HIF-2α protein, 25 nM radiolabeled tracer and 3 ug beads per well with compounds in 2% DMSO. The plate was read using a MicroBeta Microplate Counter (PerkinElmer) for luminescence detection. The IC₅₀ values of the test compounds were determined from compound dose response curves fitted using a standard four parameter fit equation and are reported in Table 1.

TABLE 1
Potency of select compounds
Less than 1 μM (+++), 1 μM to 10 μM (++), greater than 10 μM (+)

| | HIF-2α | HIF-2α |
|---|---|---|
| Example | Luciferase | Scintillation |
| # | Assay | Proximity Assay |
| 1 | ++ | n.d. |
| 2 | ++ | + |
| 3 | + | n.d. |
| 4 | + | + |
| 5 | ++ | n.d. |
| 6 | ++ | n.d. |
| 7 | + | + |
| 8 | ++ | + |
| 9 | ++ | + |
| 10 | ++ | + |
| 11 | ++ | + |
| 12 | ++ | ++ |
| 13 | ++ | + |
| 14 | ++ | ++ |
| 15 | +++ | +++ |
| 16 | ++ | + |
| 17 | ++ | + |
| 18 | ++ | + |
| 19 | ++ | + |
| 20 | + | + |
| 21 | + | + |
| 22 | − | ++ |
| 23 | ++ | ++ |
| 24 | ++ | ++ |
| 25 | +++ | +++ |
| 26 | ++ | ++ |
| 27 | ++ | +++ |
| 28 | ++ | ++ |
| 29 | + | + |
| 30 | + | ++ |
| 31 | ++ | ++ |
| 32 | + | + |
| 33 | + | + |
| 34 | + | + |
| 35 | + | ++ |
| 36 | + | ++ |
| 37 | ++ | ++ |
| 38 | ++ | ++ |
| 39 | ++ | ++ |
| 40 | ++ | +++ |
| 41 | ++ | +++ |
| 42 | ++ | +++ |
| 43 | ++ | n.d. |
| 44 | + | − |
| 45 | ++ | ++ |
| 46 | + | ++ |
| 47 | + | ++ |
| 48 | + | + |
| 49 | + | n.d. |
| 50 | + | + |
| 51 | + | + |
| 52 | + | + |
| 53 | + | + |
| 54 | + | + |
| 55 | + | + |
| 56 | + | + |
| 57 | + | ++ |
| 58 | ++ | ++ |
| 59 | ++ | + |
| 60 | ++ | + |
| 61 | + | +++ |
| 62 | + | +++ |
| 63 | + | ++ |
| 64 | + | ++ |
| 65 | ++ | n.d. |
| 66 | ++ | n.d. |
| 67 | ++ | n.d. |
| 68 | ++ | ++ |
| 69 | ++ | ++ |
| 70 | ++ | ++ |

n.d. Not determined

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

What is claimed is:

1. A compound represented by Formula (I) 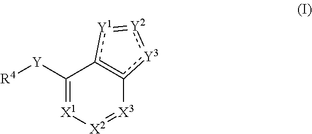
or a pharmaceutically acceptable salt thereof, wherein,
the dashed bonds are single or double bonds consistent with the groups provided for Y¹, Y² and Y³;
X¹ is CR¹ or N;
X² is CR² or N;
X³ is CR³ or N;
Y is selected from the group consisting of —O—, —C(R^{a})(R^{b})—, —N(R^{a})—, —C(R^{a})(R^{b})—N(R^{a})—, —S— and —S(O)₂—;
Y¹, Y² and Y³ are each independently selected from the group consisting of CR⁵, NR⁶ and N, wherein one of Y¹, Y² and Y³ is N, and one of Y¹, Y² and Y³ is NR⁶;
R¹ and R² are each members independently selected from the group consisting of H, halogen, CN, —NO₂, C₁₋₄ alkyl, C₁₋₄ haloalkyl and C₁₋₄ haloalkoxy;
R³ is a member selected from the group consisting of H, halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, C₁₋₄ haloalkoxy, C₆₋₁₀ aryl and 5-10 membered heteroaryl having 1 to 4 heteroatom ring vertices independently selected from the group consisting of N, O, and S;
when R¹, R² and R³ are each present, at least one is other than H;
R⁴ is a member selected from the group consisting of C₁₋₈ alkyl, C₁₋₈ alkoxy, C₃₋₈ cycloalkyl, C₆₋₁₀ aryl, and 6-membered heteroaryl having 1 to 4 heteroatom ring vertices independently selected from the group consisting of N, O, and S;
each R⁵ is a member selected from the group consisting of H, —NO₂, —S(O)₂R^{a}, —S(O)₂NR^{a}R^{b}, —S(O)(NH)R^{a}, —C(O)R^{a}, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S;
each R⁶ is a member selected from the group consisting of H, C₁₋₈ alkyl, C₆₋₁₀ aryl and 5-10 membered heteroaryl having 1 to 4 heteroatom ring vertices independently selected from the group consisting of N, O, and S;
wherein each R^{a} and R^{b} is independently selected from the group consisting of H, C₁₋₈ alkyl, C₁₋₈ alkoxy, C₁₋₈ haloalkyl, C₁₋₈ haloalkoxy, and C₁₋₈ hydroxyalkyl;
and for each R⁴, R⁵ and R⁶, each C₃₋₈ cycloalkyl, C₆₋₁₀ aryl and heteroaryl is unsubstituted or substituted with from one to five W;
wherein each R^{c} is independently selected from the group consisting of halogen, CN, —NO₂, C₁₋₈ alkyl, C₁₋₈ alkoxy, C₁₋₈ haloalkyl, —S(O)₂R^{d}, —C(O)NR^{d}R^{e} and —P(O)R^{d}R^{e};
and R^{d} and R^{e} are each independently selected from the group consisting of H, C₁₋₈ alkyl, C₁₋₈ alkoxy, C₁₋₈ haloalkyl and C₁₋₈ haloalkoxy.

2. The compound of claim 1, or a pharmaceutically acceptable salt thereof, wherein Y is —O—.

3. The compound of claim 1, or a pharmaceutically acceptable salt thereof, wherein Y is —O—; and Y¹ is CR⁵.

4. The compound of claim 1, or a pharmaceutically acceptable salt thereof, wherein Y is —O—; and Y¹ is CR⁵; and Y² is N.

5. The compound of claim 1, or a pharmaceutically acceptable salt thereof, wherein Y is —O—; Y¹ is CR⁵; Y² is N; and Y³ is NH.

6. The compound of claim 1, or a pharmaceutically acceptable salt thereof, wherein Y is —O—; Y¹ is NH; Y² is N; and Y³ is CR⁵.

7. The compound of claim 1, or a pharmaceutically acceptable salt thereof, wherein Y¹ is CR⁵; Y² is N; Y³ is NH; R³ is other than H; and each R⁵ is a member selected from the group consisting of —S(O)₂W, —S(O)₂NR^{a}R^{b}, —S(O)(NH)R^{a}, —C(O)W, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S.

8. The compound of claim 1 or a pharmaceutically acceptable salt thereof having the formula (I-ai) 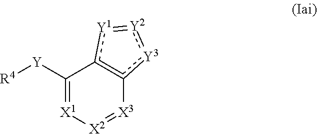
wherein, the dashed bonds are single or double bonds consistent with the groups provided for Y¹, Y² and Y³;
X¹ is CR¹ or N;
X² is CR² or N;
X³ is CR³ or N;
Y is selected from the group consisting of —O—, —C(R^{a})(R^{b})—, —N(R^{a})—, and —C(R^{a})(R^{b})—N(R^{a})—;
Y¹, Y² and Y³ are each independently selected from the group consisting of CR⁵, NR⁶ and N, wherein one of Y¹, Y² and Y³ is N, and one of Y¹, Y² and Y³ is NR⁶;
R¹ and R² are each members independently selected from the group consisting of H, halogen, and CN;
R³ is a member selected from the group consisting of H, halogen, CN, —S(O)₂R^{a}, and C₁ haloalkoxy;
when R¹, R² and R³ are each present, at least one is other than H;
R⁴ is a member selected from the group consisting of C₃₋₅ cycloalkyl, C₆ aryl, and 6-membered heteroaryl having 1-3 heteroatoms selected from O and N, wherein each of C₃₋₅ cycloalkyl, C₆ aryl, and 6-membered heteroaryl is substituted or unsubstituted with 1-3 R^{c}
each R⁵ is a member selected from the group consisting of H, CN, halogen, C₁₋₃ alkyl, C₁₋₃ alkoxy, C₁₋₃ alkoxymethyl, C₁₋₃ haloalkyl,
each R⁶ is a member selected from the group consisting of H and C₁₋₃ alkyl;
wherein each R^{a} and R^{b} is independently selected from the group consisting of H, C₁₋₃ alkyl, C₁₋₃ alkoxy, C₁₋₃ haloalkyl, C₁₋₃ haloalkoxy, and C₁₋₃ hydroxyalkyl; wherein each R^{c} is independently selected from the group consisting of F, Cl, CN, CH₃.

9. The compound of claim 1, or a pharmaceutically acceptable salt thereof having Formula (I-b): 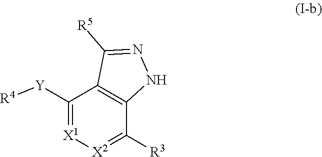
wherein
Y is selected from the group consisting of —O—, —C(R^{a})(R^{b})—, —N(R^{a})—, —C(R^{a})(R^{b})—N(R^{a})—, —S— and —S(O)₂—;
X¹ is CR¹ or N;
X² is CR² or N;
R¹ and R² are each members independently selected from the group consisting of H, halogen, CN, —NO₂ and C₁₋₄ haloalkyl;
R³ is a member selected from the group consisting of H, —NO₂, —S(O)₂R^{a}, —C(O)NR^{a}R^{b}, CN, halogen, —P(O)R^{a}R^{b}, C₁₋₈ alkyl, C₁₋₈ alkoxy, C₁₋₈ haloalkyl, C₆₋₁₀ aryl and 5-10 membered heteroaryl;
when R¹, R² and R³ are each present, at least one is other than H;
R⁴ is a member selected from the group consisting of C₁₋₈ alkyl, C₁₋₈ alkoxy, C₃₋₈ cycloalkyl, C₆₋₁₀ aryl, and 6-membered heteroaryl having 1 to 4 heteroatom ring vertices independently selected from the group consisting of N, O, and S;
each R⁵ is a member selected from the group consisting of —NO₂, —S(O)₂R^{a}, —S(O)₂NR^{a}R^{b}, —S(O)(NH)R^{a}, —C(O)R^{a}, —C(O)NR^{a}R^{b}, CN, halogen, —P(O)R^{a}R^{b}, C₁₋₈ alkyl, C₁₋₈ alkoxy, C₁₋₈ alkoxymethyl, C₁₋₈ haloalkyl, C₁₋₈ hydroxyalkyl, —NR^{a}R^{b}, C₆₋₁₀ aryl and 5-10 membered heteroaryl having 1 to 4 heteroatom ring vertices independently selected from the group consisting of N, O, and S;
wherein each R^{a} and R^{b} is independently selected from the group consisting of H, C₁₋₈ alkyl, C₁₋₈ alkoxy, C₁₋₈ haloalkyl, C₁₋₈ haloalkoxy, and C₁₋₈ hydroxyalkyl;
and each C₃₋₈ cycloalkyl, C₆₋₁₀ aryl and heteroaryl is unsubstituted or substituted with from one to five R^{c};
wherein each R^{c} is independently selected from the group consisting of halogen, CN, —NO₂, C₁₋₈ alkyl, C₁₋₈ alkoxy, C₁₋₈ haloalkyl, —S(O)₂R^{d}, —C(O)NR^{d}R^{e} and —P(O)R^{d}R^{e};
and R^{d} and R^{e} are each independently selected from the group consisting of H, C₁₋₈ alkyl, C₁₋₈ alkoxy, C₁₋₈ haloalkyl and C₁₋₈ haloalkoxy.

10. The compound of claim 9, or a pharmaceutically acceptable salt thereof wherein R⁴ is selected from the group consisting of phenyl, pyridyl, pyrimidinyl, pyrazinyl, 1,2,4-triazinyl and 1,3,5-triazinyl, each of which is unsubstituted or substituted with from one to four R^{c}.

11. The compound of claim 1, or a pharmaceutically acceptable salt thereof having Formula (I-c): 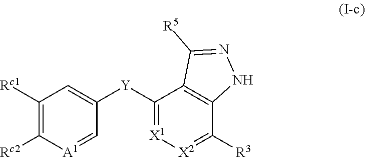
wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH;
R^{c1}, R^{c2} and R^{c3} are each independently selected from the group consisting of H, F, Cl, CN, CF₃, OCF₃ and C₁₋₄ alkyl.

12. The compound of claim 11, wherein R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are each independently selected from the group consisting of —CH₃, —CH₂CH₃, CF₃, and CHF₂.

13. The compound of claim 1, or a pharmaceutically acceptable salt thereof having Formula (I-d): 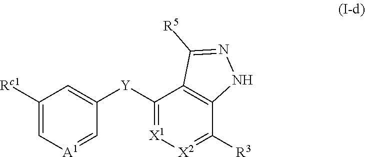
wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} is selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₄ alkyl.

14. The compound of claim 13, wherein R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are each independently selected from the group consisting of —CH₃, —CH₂CH₃, CF₃, and CHF₂.

15. The compound of claim 1, or a pharmaceutically acceptable salt thereof having Formula (I-e): 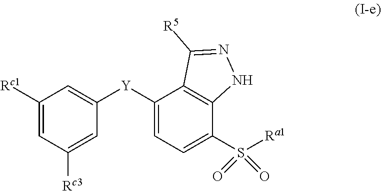
wherein
Y is —O— or —NH—;
R⁵ is selected from the group consisting of H, F, Cl, CN, I, CF₃ and CH₂OH;
R^{a1} is selected from the group consisting of CH₃, CHF₂ and CF₃; and
R^{c1} and R^{c2} are each independently selected from the group consisting of H, F, Cl, CN, CF₃, OCF₃ and C₁₋₆ alkyl.

16. The compound of claim 1, or a pharmaceutically acceptable salt thereof having Formula (I-f): 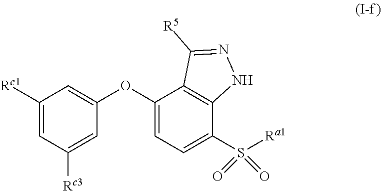
wherein
R⁵ is selected from the group consisting of H, F, Cl, CN, I, CF₃ and CH₂OH;
R^{a1} is selected from the group consisting of CH₃, CHF₂ and CF₃; and
R^{c1} and R^{c2} are each independently selected from the group consisting of H, F, Cl, CN, CF₃, OCF₃ and C₁₋₆ alkyl.

17. The compound of claim 1, or a pharmaceutically acceptable salt thereof having Formula (I-g): 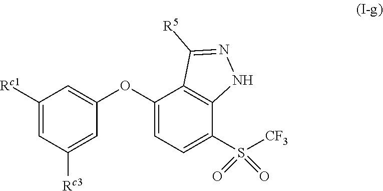
wherein
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₆ alkyl.

18. The compound of claim 1, or a pharmaceutically acceptable salt thereof having Formula (I-h): 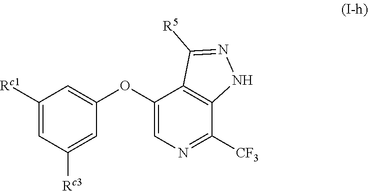
wherein
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₆ alkyl.

19. The compound of claim 1, or a pharmaceutically acceptable salt thereof having Formula (I-i): 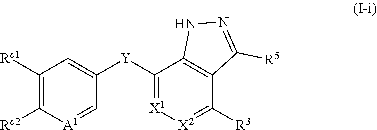
wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH; and
R^{c1}, R^{c2} and R^{c3} are each independently selected from the group consisting of H, F, Cl, CN, CF₃, OCF₃ and C₁₋₄ alkyl.

20. The compound of claim 1, or a pharmaceutically acceptable salt thereof having Formula (I-j): 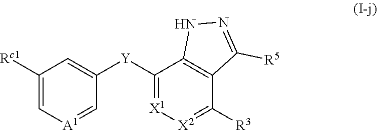
wherein
A¹ is N or CR^{c3}
Y is —O— or —NH—;
R³ is a member selected from the group consisting of halogen, CN, —NO₂, —S(O)₂R^{a}, —C(O)NR^{a}R^{b}, —P(O)R^{a}R^{b}, C₁₋₈ alkyl, C₁₋₈ alkoxy, C₁₋₈ haloalkyl, and C₁₋₄ haloalkoxy, wherein R^{a} and R^{b} are independently selected from the group consisting of C₁₋₈ alkyl, C₁₋₈ alkoxy, C₁₋₈ haloalkyl and C₁₋₄ haloalkoxy;
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₄ alkyl.

21. The compound of claim 1, or a pharmaceutically acceptable salt thereof having Formula (I-k): 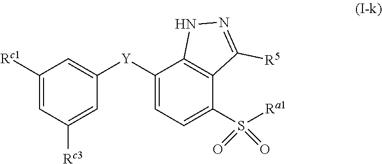
wherein
Y is —O— or —NH—;
R⁵ is selected from the group consisting of H, F, Cl, CN, I, CF₃ and CH₂OH;
R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

22. The compound of claim 1, or a pharmaceutically acceptable salt thereof having Formula (I-1): 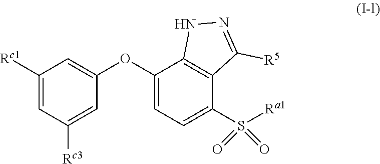
wherein
R⁵ is selected from the group consisting of H, F, Cl, CN, I, CF₃ and CH₂OH;
R^{a1} is selected from the group consisting of CH₃, CHF₂ and CF₃; and
R^{c1} and R^{c3} are each independently selected from the group consisting of H, F, Cl, CN, CF₃, OCF₃ and C₁₋₆ alkyl.

23. The compound of claim 1, or a pharmaceutically acceptable salt thereof having Formula (I-m): 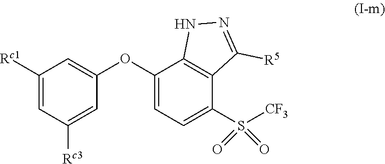
wherein
R⁵ is selected from the group consisting of H, F, Cl, CN, I, CF₃ and CH₂OH;
R^{c1} and R^{c3} are each independently selected from the group consisting of H, F, Cl, CN, CF₃, OCF₃ and C₁₋₆ alkyl.

24. The compound of claim 1, or a pharmaceutically acceptable salt thereof having Formula (I-n): 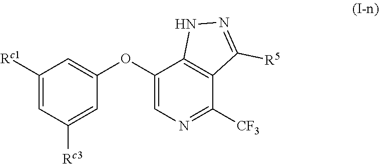
wherein
R⁵ is selected from the group consisting of H, F, Cl, CN, I, CF₃ and CH₂OH; and
R^{c1} and R^{c3} are each independently selected from the group consisting of H, F, Cl, CN, CF₃, OCF₃ and C₁₋₆ alkyl.

25. A compound selected from thegroup cosisting of:

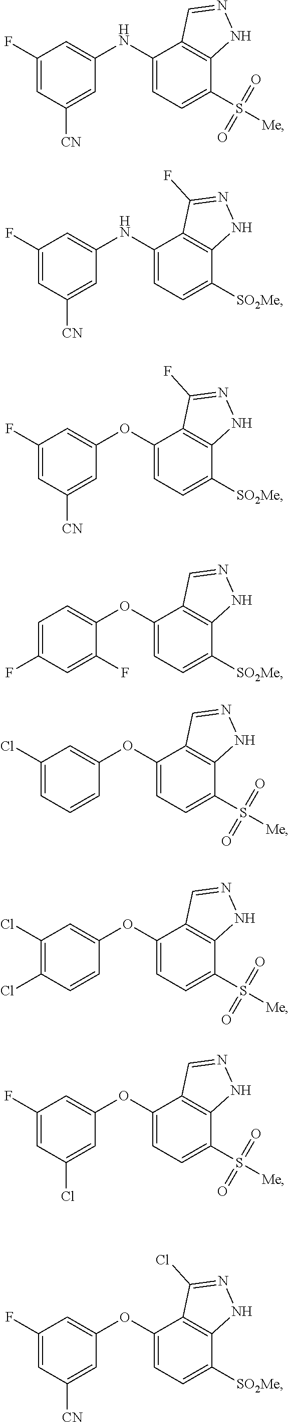

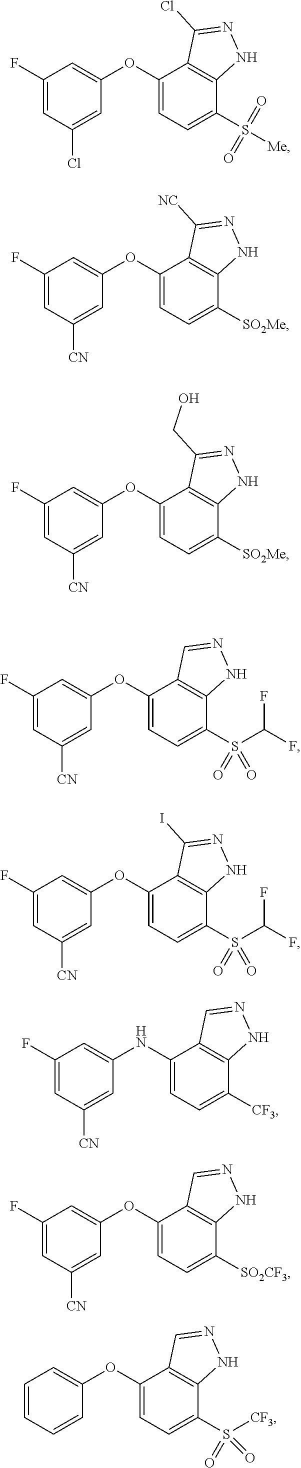

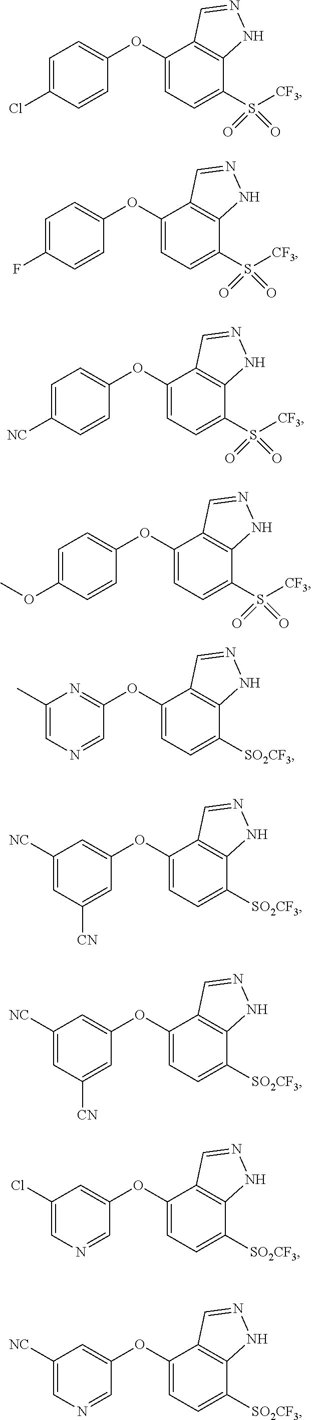

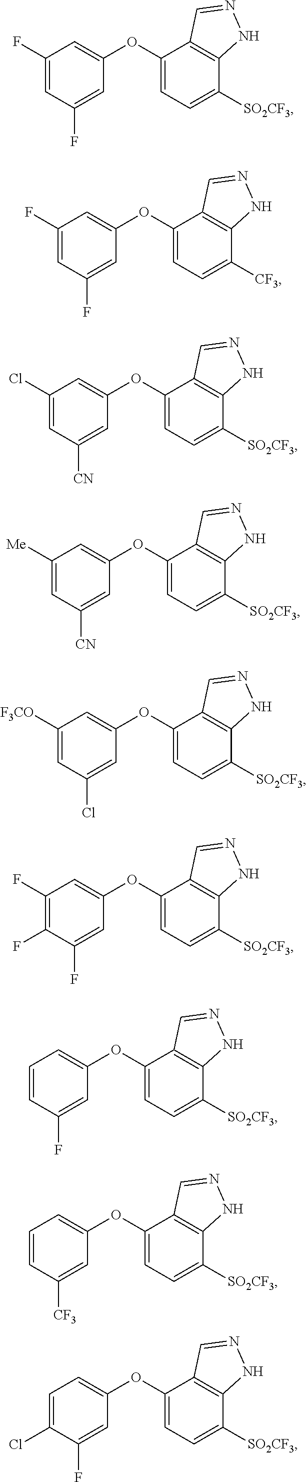

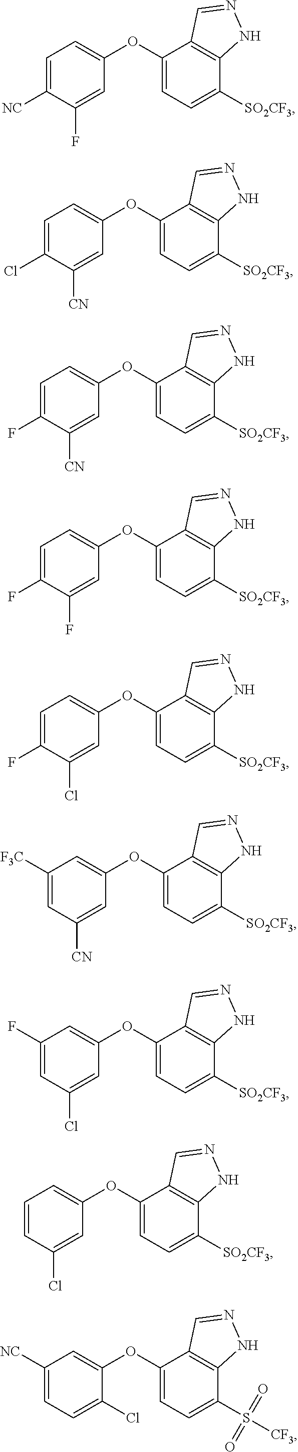

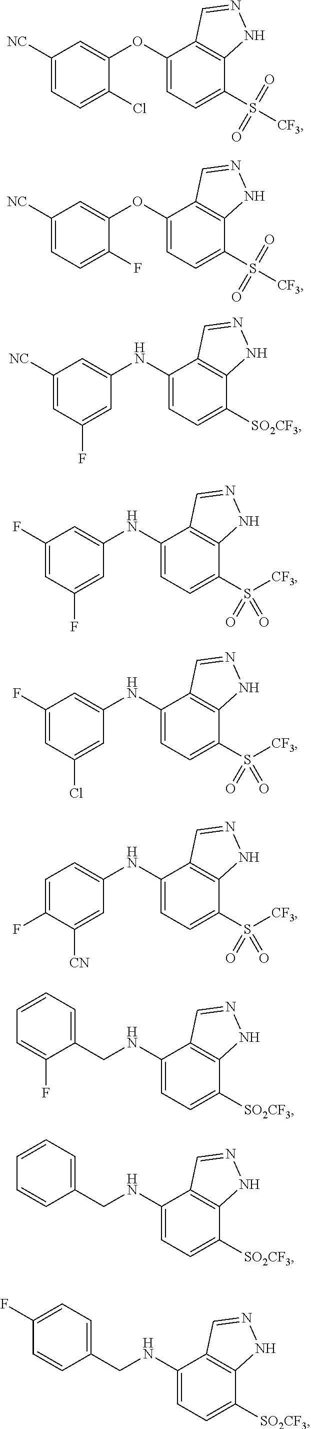

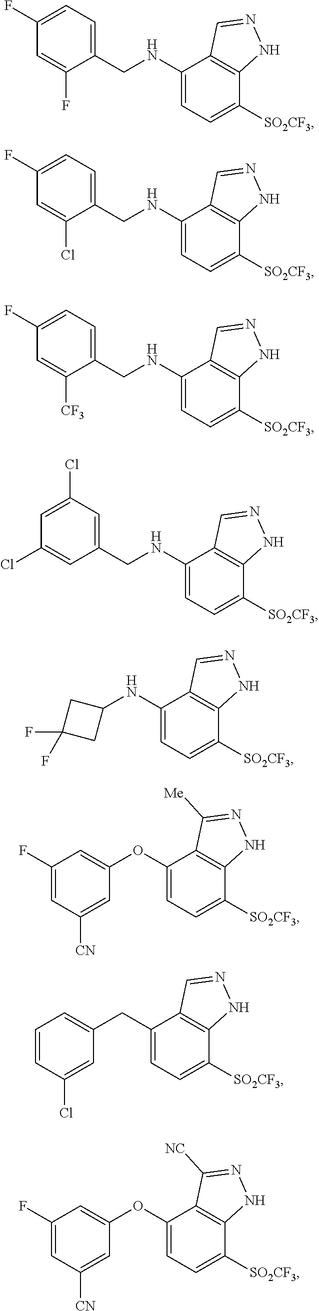

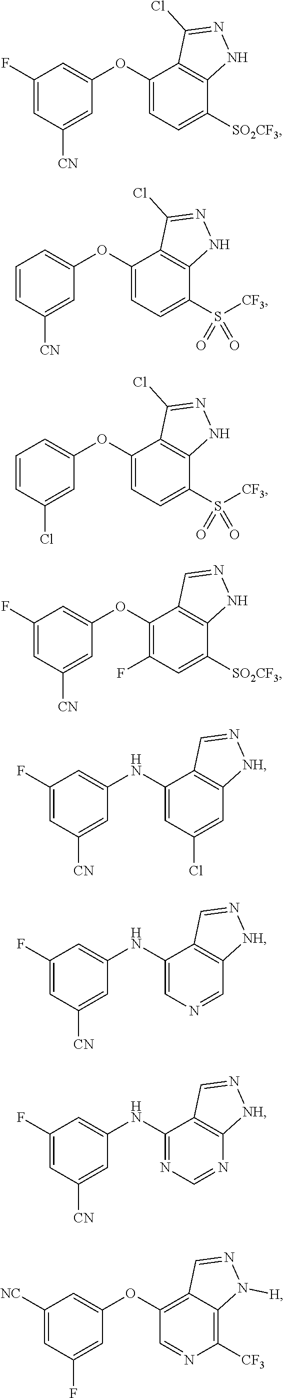

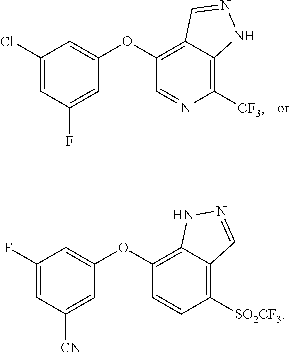

26. A pharmaceutical composition comprising a compound of any one of claims 1-25 and a pharmaceutically acceptable excipient.

27. A method of treating a disease, disorder, or condition, mediated at least in part by HIF-2α, said method comprising administering a therapeutically effective amount of a compound of any one of claims 1-25 to a subject in need thereof.

28. The method of claim 27, wherein said compound is administered in an amount effective to reverse, slow or stop the progression of HIF-2α-mediated dysregulation.

29. The method of any one of claims 27-28, wherein said disease, disorder, or condition is cancer.

30. The method of claim 29, wherein said cancer is a cancer of the prostate, colon, rectum, pancreas, cervix, stomach, endometrium, uterus, brain, liver, bladder, ovary, testis, head, neck, skin (including melanoma and basal carcinoma), mesothelial lining, white blood cell (including lymphoma and leukemia), esophagus, breast, muscle, connective tissue, intestine, lung (including small-cell lung carcinoma and non-small-cell lung carcinoma), adrenal gland, thyroid, kidney, or bone; or is glioblastoma, mesothelioma, renal cell carcinoma, gastric carcinoma, sarcoma (including Kaposi's sarcoma), choriocarcinoma, cutaneous basocellular carcinoma, or testicular seminoma.

31. The method of claim 29, wherein said cancer is selected from the group consisting of melanoma, colorectal cancer, pancreatic cancer, breast cancer, prostate cancer, lung cancer, leukemia, a brain tumor, lymphoma, ovarian cancer, Kaposi's sarcoma, renal cell carcinoma, head and neck cancer, esophageal cancer and urothelieal carcinoma.

32. The method of any one of claims 27-28, wherein said disease, disorder, or condition is an immune-related disease, disorder or condition.

33. The method of claim 32, wherein said immune-related disease, disorder, or condition is selected from the group consisting of rheumatoid arthritis, kidney failure, lupus, asthma, psoriasis, colitis, pancreatitis, allergies, fibrosis, anemia fibromyalgia, Alzheimer's disease, congestive heart failure, stroke, aortic valve stenosis, arteriosclerosis, osteoporosis, Parkinson's disease, infections, Crohn's disease, ulcerative colitis, allergic contact dermatitis and other eczemas, systemic sclerosis and multiple sclerosis.

34. The method of claim 27, further comprising at least one additional therapeutic agent.

35. The method of claim 34, wherein said at least one additional therapeutic agent is an immune checkpoint inhibitor.

36. The method of claim 35, wherein said immune checkpoint inhibitor blocks the activity of at least one of PD-1, PD-L1, BTLA, LAG3, a B7 family member, TIM-3, TIGIT or CTLA4.

37. The method of claim 36, wherein said immune checkpoint inhibitor blocks the activitiy of PD-1 or PD-L1.

38. The method of claim 37, wherein said immune checkpoint inhibitor is zimberelimab.

39. The method of claim 36, wherein said immune checkpoint inhibitor blocks the activity of TIGIT.

40. The method of any one of claims 36 to 39, further comprising a chemotherapeutic agent.

41. The method of any one of claims 36 to 40, further comprising an A2R antagonist.

42. The method of any one of claims 36 to 41, further comprising a CD73 inhibitor.

43. The method of any one of claims 36 to 42, further comprising radiation.

44. A combination comprising a compound of any one of claims 1-25, and at least one additional therapeutic agent.
